(19) United States
(12) Patent Application Publication
(10) Pub. No.: US 2022/0211737 A1
(43) Pub. Date: Jul. 7, 2022

(54) COMPOSITIONS AND METHODS FOR TREATMENT OF FRIEDREICHS ATAXIA

(71) Applicant: LACERTA THERAPEUTICS, INC., Alachua, FL (US)

(72) Inventors: Darin FALK, Alachua, FL (US); Edgardo RODRIGUEZ, Alachua, FL (US); Madhurima SAHA, Alachua, FL (US)

(73) Assignee: LACERTA THERAPEUTICS, INC., Alachua, FL (US)

(21) Appl. No.: 17/434,318

(22) PCT Filed: Sep. 11, 2020
(86) PCT No.: PCT/US2020/050551

§ 371 Date: Aug. 26, 2021

Related U.S. Application Data
(60) Provisional application No. 62/899,953, filed on Sep. 13, 2019.

(51) Int. Cl. A61K 31/7088; C12N 15/86; A61P 25/14

(52) U.S. Cl. CPC A61K 31/7088; A61P 25/14; C12N 15/86

(57) ABSTRACT

The present application provides compositions for treatment of Friedreich's Ataxia (FA). These include, but are not limited to, nucleic acid constructs and recombinant vectors comprising a human frataxin 5′ untranslated region (5′UTR FXN) and a human frataxin (FXN) nucleotide sequence are provided herein. Also provided are methods for treatment of FA.

### PRIOR RELATED APPLICATION

This application claims the benefit of and priority to U.S. Provisional Application No. 62/899,953, filed on Sep. 13, 2019, which is hereby incorporated by reference in its entirety.

### FIELD

The present disclosure generally relates to compositions and methods for treatment of Friedreich's Ataxia (FA).

### INCORPORATION BY REFERENCE OF SEQUENCE LISTING

This application contains a Sequence Listing in computer readable form (filename: 1191563seqlist.txt; created Sep. 11, 2020), which is incorporated by reference in its entirety and forms part of the disclosure.

### BACKGROUND

Friedreich's ataxia (FA) is an autosomal recessive disease and is the most common form of hereditary ataxia in the United States, affecting ˜1 in every 40,000 people. It is caused by expansion of a GAA triplet in the first intron of the FXN gene (FIG. 1B). This mutation, not present in healthy individuals (FIG. 1A) alters FIN transcription and decreases the expression of frataxin (FXN), a small mitochondrial protein involved in iron sulfur cluster assembly (Cook et al. Friedreich's ataxia: clinical features, pathogenesis, and management. Br Med Bull, 2017:124(1):19-30). Individuals who inherit two alleles with a trinucleotide repeat expansion in the FXN gene will likely develop FA.

FA is characterized by progressive degeneration of the central nervous system (CNS) leading to ataxia and is associated with heart disease (e.g. hypertrophic cardiomyopathy or myocardial fibrosis). These symptoms begin to manifest between 5 and 15 years of age and worsen over time in both males and females. However, in approximately 15% of patients, a less severe gene mutation accounts for disease onset alter the age of 25. Within 10-15 years after diagnosis, most affected individuals are confined to a wheelchair due to loss of sufficient motor control (Rummey et al, Predictors of Loss of Ambulation in Friedreich's Ataxia ECinicalMedicine. 2020 Jan. 8; 18:100213). In late stages of the disease, patients become incapacitated and generally die in early adulthood from heart disease. Currently, there is no known cure or effective treatment for FA.

### Summary

Provided herein are nucleic acid constructs for modulating expression of human frataxin in vitro, ex vivo and in vivo. The nucleic acid constructs include a nucleic acid sequence including a human frataxin 5′ untranslated region (5′UTR FXN) and a nucleic acid sequence encoding human frataxin (FXN). In some embodiments, the nucleic acid sequence encodes a human frataxin having the amino acid sequence of SEQ ID NO: 60. In some embodiments, and not to be limiting, the nucleic acid sequence encoding human FXN has at least 85% sequence identity to SEQ ID NO: 1. In some embodiments, the 5′UTR FXN has at least 85% sequence identity to SEQ ID NO: 2. In some embodiments, the 5′UTR FXN includes SEQ ID NO: 2. In some embodiments, the 5′UTR FXN includes a CCCTC-binding factor (CTCF) binding site. In certain embodiments, the CTCF binding site includes any one of SEQ ID NOs: 3 or 16-21.

In some embodiments, the nucleic acid sequence encoding human FXN is codon-optimized. In some embodiments, the 5′UTR FXN is located upstream of the nucleic acid sequence encoding human FXN.

In some embodiments, the nucleic acid construct further includes an intron, wherein the intron is positioned downstream of the 5′UTR FXN and upstream of the nucleic acid encoding human FXN.

In some embodiments the nucleic acid construct further includes a nucleic acid sequence including an RNA polymerase II promoter.

In some embodiments, the nucleic acid construct includes, in the following order:

(a) a nucleic acid sequence including a RNA polymerase II promoter; (b) a nucleic acid sequence including a 5′UTR FXN; and (c) a nucleic acid sequence encoding human FXN, wherein the nucleic acid sequence encoding human FXN has at least 85% sequence identity to SEQ ID NO: 1, and wherein the RNA polymerase II promoter is operably linked to the 5′UTR FXN and the nucleic acid sequence encoding a human FXN.

In some embodiments, the nucleic acid construct includes, in the following order: (a) a nucleic acid sequence including RNA polymerase II promoter; (b) a nucleic acid sequence including a 5′UTR FXN; (c) an intron; and (d) a nucleic acid sequence encoding human FXN, wherein the RNA polymerase II promoter is operably linked to the 5′UTR FXN and the nucleic acid sequence encoding a human FXN. In some embodiments, the nucleic acid sequence encodes a amino acid sequence comprising SEQ ID NO: 60. In some embodiments, the nucleic acid sequence encoding human FXN has at least 85% sequence identity to SEQ ID NO: 1, and

In some embodiments, the RNA polymerase II promoter is selected from the group consisting of a desmin promoter, a CBA promoter and a human frataxin promoter. In some embodiments, the RNA polymerase II promoter includes SEQ ID NO: 4 or SEQ ID NO: 5.

In some embodiments, the RNA polymerase II promoter is a spatially-restricted promoter. In some embodiments, the spatially-restricted promoter is selected from the group consisting of: a neuron-specific promoter, a cardiomyocyte-specific promoter, a skeletal muscle-specific promoter, a liver-specific promoter, astrocyte-specific promoter, microglial-specific promoter, and oligodendrocyte-specific promoter.

In some embodiments, the nucleic acid construct further includes a pair of inverted terminal repeats (ITR), wherein the nucleic acid construct is flanked on each said by an ITR.

Also provided is a recombinant viral vector including any of the nucleic acid constructs provided herein. In some embodiments, the viral vector is an adeno-associated viral (AAV) vector. In some embodiments, the AAV vector is selected from the group consisting of AAV1 serotype vectors, AAV2 serotype vectors. AAV3 serotype vectors, AAV4 serotype vectors, AAV5 serotype vectors, AAV6 serotype vectors, AAV7 serotype vectors, AAV8 serotype vectors, AAV9 serotype vectors, AAV R174 serotype vectors. AAVDJ serotype vectors and combinations and derivatives thereof. In some embodiments, the recombinant AAV vector is a single-stranded or self-complementary AAV vector.

In some embodiments, the recombinant AAV vector includes a nucleic acid sequence having at least 85% sequence identity to any one of SEQ ID NOs: 6, 14-15, or 24-28. In some embodiments, the recombinant AAV vector any one of SEQ ID NOs: 6, 14-15 or 24-28.

Also provided is a nucleic acid including any of the recombinant AAV vectors described herein. In some embodiments, the nucleic acid including the recombinant AAV vector is a plasmid.

Also provided are recombinant AAV particles including any of the recombinant AAV vectors provided herein. A plurality of any of the AAV particles described herein is also provided.

Further provided is a pharmaceutical composition including a plurality of any of the AAV particles provided herein.

Also provided are genetically modified cells including any of the nucleic acid constructs or recombinant viral vectors described herein. The genetically modified cell can be an in vitro, ex vivo or in vivo modified cell, In some embodiments, the genetically modified cell is selected from the group consisting of a human neuron, a human cardiomyocyte, a human smooth muscle myocyte, a human skeletal myocyte, and a human hepatocyte.

Also provided are methods for treating FA. The methods include administering to a subject having FA, a therapeutically effective amount of any of the recombinant AAV particles provided herein.

Also provided are methods of modulating expression of FXN in a human cell. In some embodiments, the methods include introducing into the human cell, any of the recombinant AAV vectors provided herein.

Also provided are methods for increasing adenosine triphosphate (ATP) concentration in a human cell of a subject with FA. The methods include administering to the subject a therapeutically effective amount of any of the recombinant AAV particles provided herein. In some methods, the human cell is selected from the group consisting of: a neuron, a cardiomyocyte, a smooth muscle myocyte, a skeletal myocyte, and a hepatocyte.

Also provided are methods for increasing ATP concentration in a human cell of a subject with FA. The methods include administering a therapeutically effective amount of any of the recombinant AAV particles provided herein. In some methods, the human cell is selected from the group consisting of: a neuron, a cardiomyocyte, a smooth muscle myocyte, a skeletal myocyte, and a hepatocyte.

The foregoing summary is illustrative only and is not intended to be in any way limiting. In addition to the illustrative examples and features described herein, further aspects, examples, objects and features of the disclosure will become fully apparent from the drawings, the detailed description and the claims.

### DESCRIPTION OF THE FIGURES

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIGS. 1A-1B illustrate a genomic DNA sequence and expression thereof in a healthy individual (e.g., an individual without FA) and in an individual with FA. FIG. 1A illustrates a genomic DNA sequence and expression thereof in a healthy individual. FIG. 1B illustrates a genomic DNA sequence and expression thereof in an individual with FA.

FIGS. 2A-2B illustrate exogenous nucleic acids and expression thereof in an individual with FA. FIG. 2A illustrates an exogenous nucleic acid and non-modulated expression thereof in an individual with FA. FIG. 2B illustrates an exogenous nucleic acid and modulated expression thereof in an individual with FA.

FIG. 3 illustrates an AAV vector sequence including a human FXN ORF operably linked to a 5′ITR FXN (SEQ ID NO: 2) and a RNA polymerase II promoter.

FIG. 4 shows a Western blot performed on whole cell extracts of human embryonic kidney (HEK) 293 cells.

FIG. 5 shows a Western blot performed on whole cell extracts of SK-N-SH cells

FIG. 6 shows a Western blot performed on whole cell extracts of C2C12 mouse myoblast cells.

FIG. 7 shows a bar graph showing ATP content of C2C12 mouse myoblast cells

FIG. 8 shows a Western blot performed on whole cell extracts of C2C12 mouse myoblast cells.

FIG. 9 shows a bar graph showing qPCR results of whole cells extracts of C2C12 mouse myoblast cells.

FIGS. 10A-10B are maps of different vectors, different promoters, and/or a codon-optimized sequence encoding human frataxin with and without a 5′ UTR. FIG. 10A is a map of the LP1001 AAV vector including a desmin promoter, a 5′ UTR and a codon-optimized sequence encoding human frataxin. FIG. 10B is a map of the LP1002 AAV vector including a chicken μ-actin (CBA) promoter and a codon-optimized sequence encoding human frataxin. FIG. 10C is a map of the LP1003 AAV vector including a CBA promoter, a 5′ UTR and a codon-optimized sequence encoding human frataxin. FIG. 1D is a map of the LP1004 AAV vector including a desmin (DES) promoter and a codon-optimized sequence encoding human frataxin. FIG. 10E is a map of the LP1049 AAV vector including a CBA promoter, a 3′ UTR and a codon-optimized sequence encoding human frataxin. FIG. 10F is a map of a AAV8TM vector (SEQ ID NO: 61).

FIGS. 11A-C show the toxicity of plasmid-induced FXN expression in control and patient fibroblasts, Fibroblasts were transfected with plasmids expressing FXN controlled by a CBA or DES promoter with or without a 5′ UTR. (A) Fibroblasts from normal, healthy individuals or Friedreich's ataxia patients were transfected with plasmid constructs as indicated. (B) DNA content was measured by CyQUANT Proliferation Assay to evaluate potential plasmid toxicity in fibroblast cultures. The line represents the value at which no toxicity was observed. In control fibroblasts, plasmids containing a 5′UTR showed ˜200% reduced toxicity compared to constructs without a 5′UTR. All FXN expressing plasmids showed attenuated levels of toxicity in FA patient fibroblasts. (C) To determine the effect of FXN overexpression on ATP levels, mitochondria were isolated from control and transfected fibroblasts. The line indicates the maximum obtained ATP value. Overall, ATP content was higher in fibroblast cultures treated with plasmids containing the 5′UTR compared to plasmids without a 5′UTR.(C) Means are presented by +/− SEM (n=4 wells in a 96-well plate). Statistical analysis was conducted by a two-way ANOVA followed by tukey's post hoc analysis comparing each group with one another.

FIGS. 12A-C show human FXN levels in transfected fibroblasts. (A) Western blot images and (B) quantification of images (n=2) of transfected control and diseased fibroblasts and were quantified using Gapdh as an internal control. (C) ELISA (n=2) for the detection of human FXN in transfected control and diseased fibroblasts. Results show transduction and expression in all cells treated with FXN expressing constructs. Expression levels were higher in cells transfected with plasmids lacking a 5 UTR compared to plasmids with a 5′ UTR.

FIGS. 13A-I show the results of a comparison between the 5′ untranslated region and the 3′ untranslated regions of frataxin plasmids in vitro. Fibroblasts from healthy (control) and FA patients were transfected with 5 μg of plasmid expressing FX with or without a UTR under the control of CBA promoter (Table 2). Cells that were not transfected (no plasmid) and cells transfected with CBA-GFP were used as negative and transfection control, respectively. Cells were imaged in a 24-well plate for visualization of cell confluency after transfection of constructs (FIG. 13A). Cell viability was measured after transfection by CyQUANT assay (FIG. 13B). Toxicity analyses revealed CBA-FXN decreased cell viability in control fibroblasts when compared to CBA-5′-FXN. However, FA fibroblasts do not show the same distribution of toxicity (FIG. 13C). Similarly, ATP content was measured in non- and transfected cells (FIG. 13D). Detection of frataxin overexpression by ELISA was ˜˜ 16 times higher in CBA-FXN transfected control fibroblasts above endogenous frataxin levels. CBA-5′-FXN and CBA-3-FXN were ˜10 times higher in expression when compared to endogenous frataxin expression (CBA-GFP). Densitometric analysis was performed after western blot directed against frataxin and GAPDH (FIG. 13E-3G). Immunocytochemistry detection of frataxin and tomm20 confirmed co-localization of frataxin in mitochondria (FIG. 13H) and staining of control and diseased cells in under each condition was reflective of protein expression (FIG. 13I).

FIGS. 14A-B show the titration of plasmid content to reduce the toxicity observed in vitro. To understand the potential toxicity visible in transfected fibroblasts, a dose-response (ug DNA) curve was performed. Control and FA fibroblasts were transfected with plasmids constructs expressing FXN driven by the CBA promoter (Table 2). 1×-1.25 μg, 2×-25 μg and 4×-5 μg represents the plasmid concentration of each condition. Cell toxicity analysis revealed CBA-FXN to result in the highest toxicity, however, titration of the plasmid (i.e, reduction) attenuated the degree of cell death.

FIG. 15 shows endogenous frataxin levels in wild-type nice.

FIG. 16 shows levels of human frataxin in wild-type mice following intravenous administration of AAV8TM-DES-5′UTR-FXN Intravenous AAV administration of results in significant elevation of frataxin in the heart, skeletal muscle and liver. Human FXN expression was not detected in the brain, suggesting the capsid exhibits low blood-brain barrier permeability at the dosage used.

FIGS. 17A-D show human frataxin expression in wild-type mice injected intra cisterna magna (ICM) and intramuscularly (1M) with AAV8TM-DES-5′UTR-FXN (A-C doses) Detection of human frataxin in the brain, spinal cord and skeletal muscles. (D) IHC directed against frataxin (DAB detection) shows expression throughout the medulla and pons, following ICM delivery (dose: 3e+11 vg/g of brain).

FIG. 18 shows the effects of intron placement on frataxin expression. Constructs that do not include a 5′UTR results in highly significant expression (lanes 3 and 6). Inclusion of the 5′ UTR between the intron and FXN results in low FNX expression (lane 5). Inclusion of the 5′UTR, an intron and FXN, in that order, results in desired FXN expression levels (lanes 2 and 4).

FIG. 19 shows an exemplary 5 UTR FXN sequence (SEQ ID NO: 33) with regulatory regions.

### BRIEF DESCRIPTION OF THE SEQUENCE LISTING

SEQ ID NO: 1 is an FXN nucleotide sequence which is a codon-optimized ORF from a frataxin cDNA sequence.

SEQ ID NO: 2 is an exemplary 5′ UTR FXN sequence.

SEQ ID NO: 3 is a CTCF protein binding site.

SEQ ID NO. 4 is a desmin promoter sequence.

SEQ ID NO: 5 is a chicken beta actin (CBA) promoter sequence.

SEQ ID NO: 6 is a recombinant AAV vector sequence including a codon-optimized human FXN nucleotide sequence (SEQ ID NO: 1) operably linked to a desmin promoter sequence (SEQ ID NO: 4) and further includes a desmin 5′UTR (SEQ ID NO: 22) and 5′UTR FXN sequence (SEQ ID NO: 2) operably positioned between the desmin promoter sequence and the human FXN nucleotide sequence.

SEQ ID NO: 7 is a plasmid sequence that encodes a recombinant AAV vector. The recombinant AAV vector includes a codon-optimized human FXN nucleotide sequence (SEQ ID NO: 1) operably linked to a desmin promoter sequence (SEQ ID NO: 4) and flutter includes a desmin 5′UTR (SEQ ID NO: 22) operably positioned between the desmin promoter sequence and the human FXN nucleotide sequence.

SEQ ID NO: S is a plasmid sequence that encodes a recombinant AAV vector. The recombinant AAV vector includes a codon-optimized human FXN nucleotide sequence (SEQ ID NO: 1) operably linked to a desmin promoter sequence (SEQ ID NO: 4) and further includes a desmin 5′UTR (SEQ ID NO: 22) and 5′UTR FXN sequence (SEQ ID NO: 2) operably positioned between the desmin promoter sequence and the human FXN nucleotide sequence.

SEQ ID NO: 9 is a plasmid sequence similar to SEQ ID NO: 7 except that it further includes a C-terminal V5 epitope tag in-frame with the human FXN nucleotide sequence.

SEQ ID NO: 10 is a plasmid sequence similar to SEQ ID NO: S except that it further includes a. C-terminal V5 epitope tag in-frame with the human FXN nucleotide sequence

SEQ ID NO: 11 is a plasmid sequence including a recombinant AAV vector. The recombinant AAV vector includes a codon-optimized human FXN nucleotide sequence (SEQ ID NO: 1) operably linked to a CBA promoter sequence (SEQ ID NO: 5) and further includes a CBA 5′UTR (SEQ ID NO: 23) and 5′UTR FXN sequence (SEQ ID NO: 2) operably positioned between the CBA promoter sequence and the human FXN nucleotide sequence.

SEQ ID NO: 12 is a plasmid sequence including a recombinant AAV vector. The recombinant AAV vector includes a codon-optimized human FXN nucleotide sequence (SEQ ID NO: 1) operably linked to a CBA promoter sequence (SEQ ID NO: 5) and further includes a CBA 5′UTR (SEQ ID NO: 23) operably positioned between the CBA promoter sequence and the human FXN nucleotide sequence.

SEQ ID NO: 13 is a plasmid sequence including a recombinant AAV vector. The recombinant AAV vector includes a codon-optimized human FXN nucleotide sequence (SEQ ID NO: 1) operably linked to a desmin promoter sequence (SEQ ID NO: 4) and further includes a 5′UTR FXN sequence (SEQ ID NO: 2) operably positioned between the desmin promoter sequence and the human FXN nucleotide sequence.

SEQ ID NO: 14 is a recombinant AAV vector sequence including a codon-optimized human FXN nucleotide sequence (SEQ ID NO: 1) operably linked to a CBA promoter sequence (SEQ ID NO: 5) and further includes a CBA 5′UTR (SEQ ID NO: 23) and 5′UTR FXN sequence (SEQ ID NO: 2) operably positioned between the CBA promoter sequence and the human FXN nucleotide sequence.

SEQ ID NO: 15 is a recombinant AAV vector sequence including a codon-optimized human FXN nucleotide sequence (SEQ ID NO: 1) operably linked to a desmin promoter sequence (SEQ ID NO: 4) and further includes a 5′UTR FXN sequence (SEQ ID NO: 2) operably positioned between the desmin promoter sequence and the human FXN nucleotide sequence.

SEQ ID NOs: 16-21 are CTCF protein binding sites.

SEQ ID NO: 22 is a desmin 5′UTR.

SEQ ID NO: 23 is a CBA 5′UTR.

SEQ ID NO: 24 is a plasmid sequence including a recombinant AAV vector (LP1001). The recombinant AAV vector includes, in the following order, in operable linkage, a desmin promoter sequence (SEQ ID N: 4), a 5′UTR FXN sequence (SEQ ID NO: 2), an intron, a codon-optimized human FXN nucleotide sequence (SEQ ID NO: 1).

SEQ ID NO: 25 is a plasmid sequence including a recombinant AAV vector (LP1002). The recombinant AAV vector includes, in the following order, in operable linkage, a CMV promoter sequence (SEQ ID NO: 34), a CBA promoter, (SEQ ID NO: 5, an intron and a codon-optimized human FXN nucleotide sequence (SEQ ID NO: 1).

SEQ ID NO: 26 is a plasmid sequence including a recombinant A-AV vector (LP1003). The recombinant AAV vector includes, in the following order, in operable linkage, a CMV promoter sequence (SEQ ID NO: 34), a CBA promoter. (SEQ ID NO: 5, a 5′UTR FXN sequence (SEQ ID NO: 2), an intron, and a codon-optimized human FXN nucleotide sequence (SEQ ID NO: 1).

SEQ ID NO: 27 is a plasmid sequence including a recombinant AAV vector (LP1004). The recombinant AAV vector includes, in the following order, in operable linkage, a desmin promoter sequence (SEQ ID NO: 4) an intron and a codon-optimized human FXN nucleotide sequence (SEQ ID NO: 1).

SEQ ID NO: 28 is a plasmid sequence including a recombinant A-AV vector (LP1049). The recombinant AAV vector includes, in the following order, in operable linkage, a CMV promoter sequence (SEQ ID NO: 34), a CBA promoter sequence (SEQ ID NO: 5) an intron, a codon-optimized human FXN nucleotide sequence (SEQ ID NO: 1) and a 3′ UTR FXN (SEQ ID NO: 35).

SEQ ID NO: 29 is a self-complementary plasmid sequence including AAV-CBA-EGFP (GenBauk: Accession No. MK225672).

SEQ ID NO: 30 is a primer.

SEQ ID NO: 31 is a primer.

SEQ ID NO: 32 is a primer.

SEQ ID NO: 33 is an exemplary 5′ TR FXN sequence with TFAP2 (SEQ ID NO: 57), SRF1 (SEQ ID NO: 56) and SP1 (SEQ ID NO: 58) regulatory regions.

SEQ ID NO: 34 is a CMV enhancer sequence.

SEQ ID NO: 35 is 3′ UTR FXN

SEQ ID NO: 36 is an intron sequence included in SEQ ID NO: 24.

SEQ ID NO: 37 is a modified SV40 intron with splice donor and acceptor sites.

SEQ ID NO: 38 is an exemplary mutated ITR sequence.

SEQ ID NO: 39 is an exemplary ITR sequence.

SEQ ID NO: 40 is a human 3′ UTR FXN.

SEQ ID NO: 41 is a truncated 3′UTR FXN.

SEQ ID NO: 42 is an exemplary frataxin promoter sequence.

SEQ ID NO: 43 is an exemplary frataxin promoter sequence.

SEQ ID NO: 44 is an ampicillin resistance gene.

SEQ ID NO: 45 is a kanamycin resistance gene.

SEQ ID NO: 46 is an exemplary 3′ UTR FXN sequence that does not include a putative iron binding domain.

SEQ ID NO: 47 is an exemplary 3′ UTR FXN sequence that does not include a mitochondrial localization signal.

SEQ ID NO: 48 is an exemplary 5′ UTR FXN sequence that does not include a L2 retrotransposable element (SEQ ID NO: 54).

SEQ ID NO: 49 is an exemplary 5′ UTR FXN sequence that does not include an alternate RNA export signal (SEQ ID NO: 55)

SEQ ID NO: 50 is an exemplary 5′ UTR FXN sequence that does not include a CTCF domain.

SEQ ID NO: 51 is an exemplary 5′ UTR FXN sequence.

SEQ ID NO: 52 is an exemplary 5′ UTR FXN sequence.

SEQ ID NO: 53 is an exemplary 5′ UTR FXN sequence that does not include the catalytic binding domain (SEQ ID NO: 59).

SEQ ID NO: 54 is an L2 retrotransposable element

SEQ ID NO: 55 is an alternate RNA export signal.

SEQ ID NO: 56 is an SRF regulatory sequence.

SEQ ID NO: 57 is a TFAP2 regulatory sequence.

SEQ ID NO: 58 is a regulatory SP1 sequence.

SEQ ID NO. 59 is an aconitase binding domain.

SEQ ID NO. 60 is an exemplary amino acid sequence for human frataxin.

SEQ ID NO: 61 is an AA8V triple-capsid mutant vector sequence.

SEQ ID NO: 62 is an exemplary 5′ UTR FXN.

SEQ ID NO: 63 is a nucleic acid sequence encoding a bovine growth hormone polyaclenlyation sequence.

### DETAILED DESCRIPTION

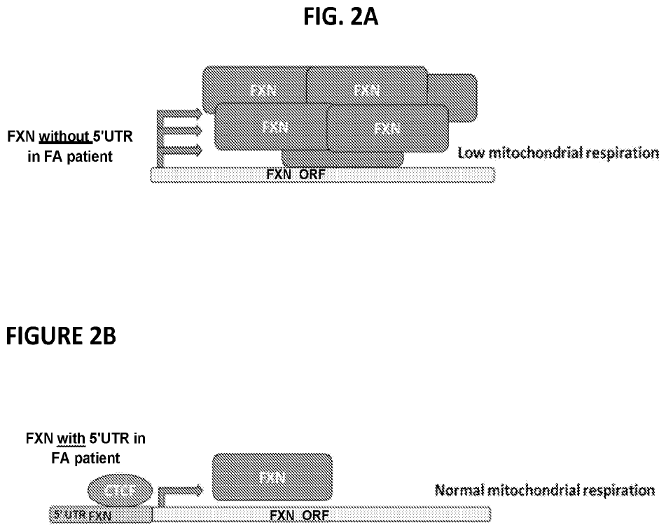

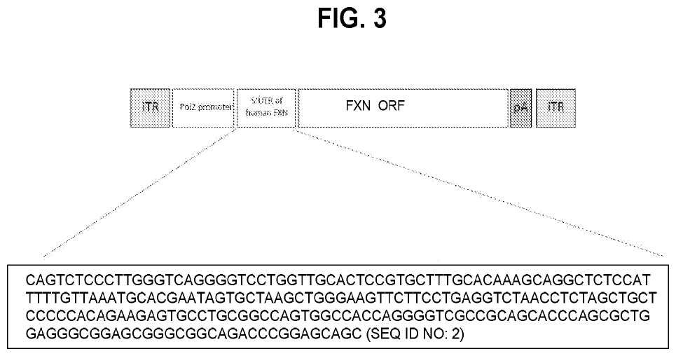

Current approaches for restoring frataxin expression focus on using non-modulated, highly-active promoter sequences to express high levels of frataxin (FIG. 2A). However, risks are associated with this approach. Non-modulated, elevated physiological levels of frataxin result in low mitochondrial respiration, which can lead to mitochondrial toxicity. In fact, reports have shown that overexpression of FXN is toxic both in vitro (Vannocci et al. “Adding a temporal dimension to the study of Friedreich's ataxia: the effect of frataxin overexpression in a human cell model,” Dis Model Mech. 2018; 11(6):dmm032706) and in viva (Belbellaa et al. “High levels of frataxin overexpression leads to mitochondrial and cardiac toxicity in mouse models,” April 2020. doi.org?10.1101/2020.03.31.015255; Belbella et al. “Correction of half the cardiomyocytes fully rescue Friedreich ataxia mitochondrial cardiomyopathy through cell-autonomous mechanisms,” Hum Mol Genet. 2019; 28(8):1274-1285).

To achieve modulated physiological levels of FXN expression in FA patients, the inventors identified the 5′ UTR of FXN as a regulatory sequence that modulates FXN expression and avoids the toxicity associated with elevated physiological levels of FXN expression. Provided herein are compositions for use in methods of treating FA in a subject. These compositions include, but are not limited to, novel nucleic acid constructs, recombinant viral vectors and cells including a human frataxin 5′UTR (5′UTR FXN) and a nucleic acid sequence encoding FXN.

### Nucleic Acid Constructs

Provided herein are nucleic acid constructs including a human frataxin 5′ untranslated region (5′UTR FXN) and a nucleic acid sequence encoding human frataxin (FXN). In some embodiments, the nucleic acid sequence encoding human FXN has at least 85% sequence identity to SEQ ID NO: 1 (as set forth below). In some embodiments, the nucleic acid sequence encoding human FXN is not a naturally-occurring nucleotide sequence encoding human FXN.

(SEQ ID NO: 1)
atgtggacat tggggcggag ggcagtggcg ggtcttcttg
cgtctcccag cccagcacaggcacaaacat tgactagagt
tccccggcca gcggagttgg cccctctctg
tggacggcggggactgcgga cggatataga cgccacctgc
acacctcgaa gagctagttc aaatcagcggggcctcaatc
aaatctggaa cgttaagaag cagagtgtgt accttatgaa
cttgagaaaaagcggaaccc tcggccaccc agggtcattg
gatgaaacaa cctatgagag gcttgcggaagagacattgg
atagcttggc cgaattcttt gaagaccttg ccgacaaacc
ctatacatttgaggattacg atgtctcctt cggctctggt
gtcctgactg tgaagttggg gggcgacctcggaacgtacg
taataaataa gcagactccg aataaacaaa tttggttgtc
ctcaccaagtagcggcccca agcggtatga ttggactggg
aagaactggg tatactccca cgacggcgttagcctgcacg
aactgttggc agccgagctt acaaaagctt tgaagacaaa
actggacctc

### 5′UTR FXN

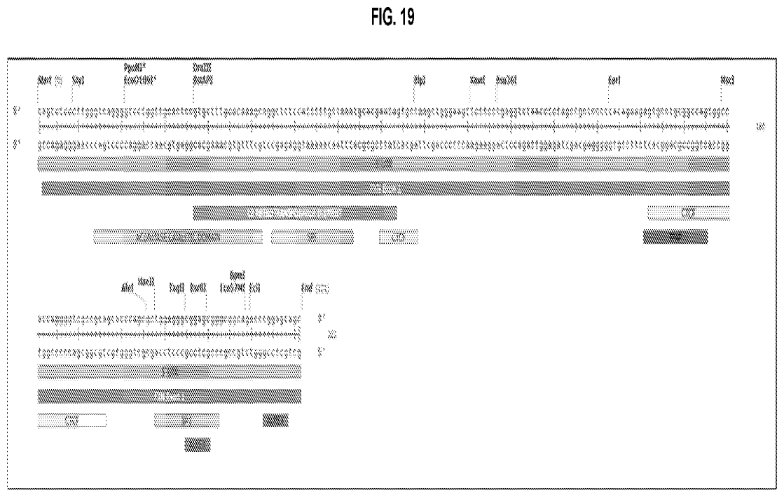

As used throughout, a 5′UTR FXN is an untranslated nucleic acid sequence, that is upstream from the initiation codon of a nucleic acid encoding human FXN. As set forth herein, a 5′ UTR FXN can modulate FXN expression. Modulated FN expression may be desired to achieve modulated physiological levels of FXN expression and mitochondrial respiration and thereby avoid non-modulated, elevated physiological levels of FX expression and reduced mitochondrial respiration. Without wishing to be bound by theory, it is believed that this modulation can be achieved via cis effects of the 5′ UTR FXN on transcription and/or translation of mRNA transcribed from a nucleic acid of the present disclosure encoding the 5′ UTR and the human FXN nucleotide sequence. The regulatory elements within the 5′ UTR FXN are shown in FIG. 19 (SEQ ID NO: 33).

The 5′UTR FXN can include a nucleotide sequence having at least 85%, 86%, 87%, 88%, 89%, 90%, 91%, 92%, 93%, 94%, 95%, 96%, 97%, 98%, or 99% sequence identity to SEQ ID NO: 2 (as set forth below) or a fragment thereof.

(SEQ ID NO: 2)
CAGTCTCCCTTGGGTCAGGGGTCCTGGTTGCACTCCGTGCTTTGCACAA
AGCAGGCTCTCCATTTTTGTTAAATGCACGAATAGTGCTAAGCTGGGAA
GTTCTTCCTGAGGTCTAACCTCTAGCTGCTCCCCCACAGAAGAGTGCCT
GCGGCCAGTGGCCACCAGGGGTCGCCGCAGCACCCAGCGCTGGAGGGCG
GAGCGGGCGGCAGACCCGGAGCAGC

The 5′UTR FXN can include a nucleotide sequence including SEQ ID NO: 2 or a fragment thereof. In other examples, the 5′UTR FXN can include SEQ ID NO: SEQ ID NO: 33, SEQ ID NO: 48. SEQ ID NO: 49. SEQ ID NO: 50. SEQ ID NO: 51, SEQ ID NO: 52, SEQ ID NO: 53, or SEQ ID NO: 62. The fragment can be at least about 2, 3, 4, 5, 6, 7, &, 9, 10, 20, 30, 40, 50 or more nucleotides shorter, at either or both ends of SEQ ID NO: 2. In some embodiments, the nucleic acid sequence including the 5′ UTR FXN is not a full-length FXN promoter. In some embodiments, the nucleotide sequence including the 5′ UTR FXN does not include, SEQ ID NO: 42 or SEQ ID NO: 43. In some embodiments, the nucleic acid sequence including the 5′ UTR FXN is a nucleic acid sequence that includes SEQ ID NO: 2, SEQ ID NO: 33, SEQ ID NO: 48, SEQ ID NO: 49. SEQ ID NO: 50. SEQ ID NO: 51, SEQ ID NO 52, SEQ ID NO: 53 and is at least 10, 20, 30, 40, 50, 60, 70, 80, 90, 100, 200, 300, 400, 500, 600, 700, 800, or 900 base pairs shorter in length, on either end, than SEQ ID NO: 43. In some embodiments, the nucleic acid sequence including the 5′ UTR FXN, includes SEQ ID NO: 2 and is not a nucleic acid sequence that is at least 10, 20, 30, 40, 50, 60, 70, 80, 90, 100, 200, 300, 400, 500, 600, 700, 800, or 900 base pairs shorter in length, on either end, than SEQ ID NO: 42.

The 5′UTR FXN can be located upstream of the nucleic acid sequence encoding human FXN for example, a nucleic acid sequence encoding SEQ ID NO: 60. The 5′UTR FXN can include a CTCF binding site. The CTCF binding site can include a nucleotide sequence including at least 85%, 86%, 87%, 88%, 89%, 90%, 91%, 92%, 93%, 94%, 95%, 96%, 97%, 98%, or 99% sequence identity to at least one of SEQ ID NOs: 3 or 16-21. The CTCF binding site can include at least one of SEQ ID NOs: 3 or 16-21. The 5′UTR FXN can include at least one CTCF binding site including any one of SEQ ID NOs: 3 and 16-21. In some embodiments, the 5′ UTR FXN does not include a functional CTCF binding site, e.g., the CTCF binding site is mutated or removed from the 5′ UTR FXN. An exemplary 5′UTR FXN that does not include a functional CTCF binding site is set forth herein as SEQ ID NO: 50.

As used herein, “modulated physiological levels of FXN expression” refers to levels of FXN expression at the protein level which are similar to those observed in wild-type cells. For example, “modulated physiological levels of FXN expression” in muscle- or nerve-derived cells including homozygous GAA repeat expansion FXN alleles treated according to methods of the present disclosure can display FXN expression levels similar to wild-type cells of a similar or isogenic background. Such “modulated physiological levels of FXN expression” can reduce negative effects on cellular mitochondria function in diseased cells due to a lack of sufficient FXN or due to a harmful excess of FXN, such as an excess due to non-modulated expression of the FXN gene.

“Modulated physiological levels of FXN expression” at the protein level can be similar to that of wild-type cells. For example, the modulated physiological levels of FXN expression at the protein level can be at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%½ at least 195%, or at least 200% of the FXN protein level in wild-type cells.

In some embodiments, including a 5′UTR FXN in a nucleic construct (e.g., a construct including a promoter, a 5′ UTR FXN and a nucleic acid encoding human FXN) results in a level of FXN expression in a cell that is at least about 5%, 10%, 15%, 20%, 25%, 30%, 35%, 40%, 45%, 50%, 60%, 70%, 80% or 90% lower than the level of FXN expression in a cell with a nucleic acid construct that does not include a 5′ UTR FXN (e.g., a construct including a promoter and a nucleic acid encoding human FXN).

### Human Frataxin (FXN Nucleotide Sequence

As used herein, “a nucleic acid sequence encoding human FXN” or a human FX nucleotide sequence” can be a human frataxin FXN cDNA sequence (e.g., SEQ ID NO: 1). Any nucleic acid sequence encoding human FXN can be operably linked to a 5′ UTR FXN described herein, including naturally and non-naturally occurring nucleic acids that encode human FXN. In some embodiments, the nucleic acid sequence encodes SEQ ID NO: 60, or SEQ ID NO: 60 with one more conservative substitutions. SEQ ID NO: 1 is an exemplary codon-optimized nucleic acid sequence that encodes human frataxin protein (SEQ ID NO: 60). An exemplary amino acid sequence for human frataxin can also be found under GenBank Accession No. NP_000135.2.

(SEQ ID NO: 60)
mwtlgrrava gllaspspaq aqtltrvprp aelaplcgrr
glrtdidatc tprrassnqr glnqiwnvkk qsvylmnlrk
sgtlghpgsl dettyerlae etldslaeff edladkpytf
edydvsfgsg vltvklggdl gtyvinkqtp nkqiwlssps
sgpkrydwtg knwvyshdgv slhellaael tkalktkldl
sslaysgkda

In some embodiments, the FXN nucleotide sequence can have at least 85%, 86%, 87%, 88%, 89% 90%, 91%, 92%, 93%, 94%, 95%, 96%, 97%, 98%, or 99% sequence identity to SEQ ID NO: 1. The FXN nucleotide sequence can include SEQ ID NO: 1. In some embodiments, the FXN nucleotide sequence is codon-optimized for expression in the cell to be infected by any of the recombinant AAV vectors or particles described herein. For example, if the recombinant AAV infected cell is a human cell, it is contemplated that a human codon-optimized polynucleotide encoding FXN, for example, SEQ ID NO: 1, can be used for producing the FXN polypeptide. Methods for codon-optimization are known in the art. See, for example, Inouye et al. “Codon optimization of genes for efficient protein expression in mammalian cells by selection of only preferred human codons,” Protein Expression and Purification 109: 47-54 (2015)). GeneOptimizer® software (Thermo Fisher Scientific, Waltham, Mass.) can also be used.

### Promoter

Also provided is a nucleic acid construct including a promoter operably linked to a nucleic acid sequence described herein. In some embodiments, the components or elements of the constructs described herein are operably linked to make a non-naturally occurring construct. In other words, the elements are not linked as they would be linked in the genome of naturally occurring cell.

Numerous promoters can be used in the constructs described herein. A promoter is a region or a sequence located upstream and/or downstream from the start of transcription that is involved in recognition and binding of RNA polymerase and other proteins to initiate transcription. In some embodiments, the promoter is an RNA polymerase II promoter, for example, and not to be limiting, an RNA polymerase II CORE promoter. As used herein, an RNA polymerase II CORE promoter is the minimal sequence that allows the basal transcription apparatus to assemble. For example, this sequence can be at 40 base pairs in length and can include a TATA box, an initiator element (Inr) and/or a downstream promoter element (DPE). See, for example, Domenger and Grimm, “Next generation AAV vectors-do not judge a virus (only) b yits cover,” Human Mol. Genetics 29(R1): R3-R14 (2019). In some embodiments the promoter is an inducible promoter, for example, the promoter can be chemically or physically regulated. A chemically regulated promoter and/or enhancer can, for example, be regulated by the presence of alcohol, tetracycline, a steroid, or a metal. Examples include, the tetracycline inducible promoter or a glucocorticoid inducible promoter. The nucleic acids of the present invention can also be under the control of a tissue-specific promoter to promote expression of the nucleic acid in specific cells, tissues or organs. Any regulatable promoter, such as a metallothionein promoter, a heat-shock promoter, and other regulatable promoters, of which many examples are well known in the art are also contemplated. Furthermore, a Cre-1oxP inducible system can also be used, as well as a Flp recombinase inducible promoter system, both of which are known in the art.

As used herein, the terms “operably linked,” “operably positioned,” and the like mean that a first nucleic acid sequence (e.g. a coding sequence for a protein or a non-coding RNA sequence) is covalently connected to at least a second nucleic acid sequence such that at least one of the two sequences can exert an effect on the other nucleic acid sequence. For example, a human FXN nucleotide sequence can be operably linked to a promoter sequence such that the promoter sequence can direct transcription of the human FXN nucleotide sequence, thereby contributing to expression of the human FXN nucleotide sequence. Similarly, a 5′ UTR FXN sequence can be operably positioned between the promoter sequence and the human FXN nucleotide sequence, such that the 5′ UTR FXN sequence can modulate expression of the human FXN nucleotide sequence.

In some embodiments, the nucleic acid construct further includes a nucleic acid sequence including an RNA polymerase II promoter that is operably linked to a 5′ UTR FXN and the nucleic acid sequence encoding a human FXN. The RNA polymerase II promoter can be, for example, a desmin promoter sequence (SEQ ID NO: 4), a CBA promoter sequence (SEQ ID NO: 5) or a frataxin promoter sequence, for example, SEQ ID NO: 42, SEQ ID NO: 43, or a fragment thereof. The RNA polymerase II promoter can include SEQ ID NO: 4. The RNA polymerase II promoter can include SEQ ID NO: 5. The RNA polymerase HI promoter can include SEQ ID NO: 42 or SEQ ID NO: 43. The RNA polymerase II promoter can include at least 85%, 86%, 87%, 88%, 89%, 90%, 91%, 92%, 93%, 94%, 95%, 96%, 97%, 98%, or 99% sequence identity to SEQ ID NO: 4. The RNA polymerase II promoter can include at least 85%, 86%, 87%, 88%, 89%, 90%, 91%, 92%, 93%, 94%, 95%, 96%, 97%, 98%, or 99% sequence identity to SEQ ID NO: 5. The RNA polymerase II promoter can include at least 85%, 86%, 87%, 88%, 89%, 90%, 91%, 92%, 93%, 94%, 95%, 96%, 97%, 98%, or 99% sequence identity to SEQ ID NO: 42 or SEQ ID NO: 43.

In some embodiments the RNA polymerase II promoter operably linked to a 5″ UTR FXN is not the endogenous promoter that is associated with the 5′ UTR FXN, i.e., it is derived from a different protein, for example, a desmin or a CBA promoter. In some embodiments, the RNA polymerase II promoter that is operably linked to a 5′ UTR FXN and the nucleic acid sequence encoding a human FXN, is not a frataxin promoter, In some embodiments, the promoter operably linked to the 5′ UTR FXN does not include SEQ ID NO: 42. SEQ ID NO: 43, or the complement of either sequence. In some embodiments, any of the construct described herein does not include a human frataxin promoter (e.g., SEQ ID NO: 42 or SEQ ID NO: 43) or a 3′ UTR FXN (e.g., SEQ ID NO: 40 or SEQ ID NO: 41).

It is understood that fragments of the desmin, CBA or a frataxin promoter can also be used in the constructs described herein, as long as the fragment retains at least 75%, 80% 85%, 90%, 95%, 100% or more of at least one activity of the promoter from which the fragment was derived, for example, the promotion of transcription of a nucleic acid in a cell (e.g., a neuronal or muscle cell. The fragment can be at least 10, 20, 30, 40, 50, 60, 70, 80, 90, 100, 150, 200, 300, 400, 500 or more nucleotides shorter than a wild-type promoter or a promoter sequence having at least 85% identity to a wild-type promoter sequence. For example, fragments that are at least 10, 20, 30, 40, 50, 100, 200, 300, 400, or 500 base pairs shorter in length than SEQ ID NO: 4, SEQ ID NO: 5, SEQ ID NO: 42 or SEQ ID NO: 43 can be used as a promoter.

In some embodiments, a 5′UTR of a RNA polymerase II promoter can be removed from any of the promoters or constructs of the present disclosure to further modulate expression of the human FXN nucleotide sequence. SEQ ID NO: 4 and SEQ ID NO: 5 are examples of a desmin promoter and a CBA promoter respectively, that do not include a 5′ UTR.

In some embodiments, an enhancer sequence, for example a CMV enhancer (e.g. SEQ ID NO: 34) is operably linked to the promoter. In some embodiments, where a CMV enhancer is operably linked to a promoter, for example, a CBA promoter, and an intron, the promoter is referred to as a CAG promoter. Exemplary constructs including a CMV enhancer, a CBA promoter and an intron are provided as SEQ ID NO: 25 and SEQ ID NO: 26.

In some embodiments, the RNA polymerase II promoter is a spatially-restricted promoter, for example, a tissue- or cell-specific promoter. The spatially-restricted promoter can be any suitable promotor, such as those selected from the group consisting of a neuron-specific promoter, a cardiomyocyte-specific promoter, a skeletal muscle-specific promoter, a liver-specific promoter, astrocyte-specific promoter, microglial-specific promoter, and oligodendrocyte-specific promoter. As used herein, specific expression does not mean that the expression product is expressed only in a specific tissue(s) or cell type(s), but refers to expression substantially limited to specific tissue(s) or cell types(s). See for example, Pacak et al. “Tissue specific promoters improve specificity of AAV9 mediated transgene expression following intra-vascular gene delivery in neonatal mice.” Genetic Vaccines and Therapy 6(13) doi:10.1186/1479-0556-6-13 (2008).

In some embodiments, the RNA polymerase II promoter can be operably linked to a 5′UTR of the RNA polymerase II promoter, the 5′UTR FXN and the FXN nucleotide sequence in the following exemplary order: RNA polymerase II promoter—5′UTR of the RNA polymerase II promoter—5′UTR FXN—nucleotide sequence. For example, the nucleic acid construct can include a codon-optimized human FXN nucleotide sequence (SEQ ID NO: 1) operably linked to a desmin promoter sequence (SEQ ID NO: 4) and further include a desmin 5′UTR (SEQ ID NO: 22) and 5′UTR FXN sequence (SEQ ID NCO: 2) operably positioned between the desmin promoter sequence and the human FXN nucleotide sequence.

The nucleic acid construct can also include a codon-optimized human FXN nucleotide sequence (SEQ ID NO: 1) operably linked to a CBA promoter sequence (SEQ ID NO: 5) and further include a CBA 5′UTR (SEQ ID NO: 23) and 5′UTR FXN sequence (SEQ ID NO: 2) operably positioned between the CBA promoter sequence and the human FXN nucleotide sequence.

In other constructs, the RNA polymerase II promoter can be operably linked to the 5′UTR FXN and the FXN nucleotide sequence in the following exemplary order: RNA polymerase II promoter—5′UTR FXN—nucleotide sequence. For example, the nucleic acid construct can include a codon-optimized human FXN nucleotide sequence (SEQ ID NO: 1) operably linked to a desmin promoter sequence (SEQ ID NO: 4) and further include a 5′UTR FXN sequence (SEQ ID NO: 2) operably positioned between the desmin promoter sequence and the human FXN nucleotide sequence. The nucleic acid construct can also include a codon-optimized human FXN nucleotide sequence (SEQ ID NO: 1) operably linked to a CBA promoter sequence (SEQ ID NO: 5) and further include a 5′UTR FXN sequence (SEQ ID NO: 2) operably positioned between the CBA promoter sequence and the human FXN nucleotide sequence.

### Introns

Any of the nucleic acid construct described herein can further include one or more intron nucleotide sequences. The intron can be located in any suitable location within the nucleic acid construct to modulate expression. The intron sequence can be located upstream of the 5′UTR FXN. The intron can be located downstream of the 5′UTR FXN. In some embodiments, the intron is positioned between the 5′UTR FXN and the nucleic acid sequence encoding human FXN. The intron, and splicing thereof, can contribute to expression of the human FXN nucleotide sequence. SEQ ID NO: 36 and SEQ ID NO: 37 (as set forth below) are exemplary intron sequences that can be used in any of the constructs provided herein. Other intron sequences are known in the art. See for example, Domrenger and Grimmn; and Huang et al. “Intervening sequences increase efficiency of RNA 3′ processing and accumulation of cytoplasmic RNA,” Nucleic Acids Res. 18(4): 937-947 (1990);

(SEQ ID NO: 36)
gtaagtatcaaagtatcaaggttacaagacaggtttaaggagaccaatag
aaactgggcttgtcgagacagagaagactcttgcgtttctgataggcacc
tattggtcttactgacatccactttgcctttctctccacag.
(SEQ ID NO: 37)
gtaagtttagtctttttgtcttttatttcaggtcccggatccggtggtgg
tgcaaatcaaagaactgctcctcagtggatgttgcctttacttctag.

In some embodiments, the intron is an intron that is not found in a naturally occurring nucleic acid encoding human frataxin.

For example, and not to be limiting, provided herein is a nucleic acid construct including, in the following exemplary order: (a) a nucleic acid sequence including RNA polymerase II promoter; (b) a nucleic acid sequence including a 5′UTR FX; (c) an intron; and (d) a nucleic acid sequence encoding human FXN, wherein the RNA polymerase II promoter is operably linked to the 5′UTR FXN and the nucleic acid sequence encoding a human FXN. In some embodiments, the nucleic acid sequence encoding, human FC has at least 85% sequence identity to SEQ ID NO: 1.

In some embodiments, the nucleic acid construct further includes a human frataxin 3′UTR (3′ UTR FXN) or a truncated 3′ UTR FXN positioned downstream of the coding sequence of human FXN. In some embodiments, the nucleic acid construct does not include a human frataxin 3′UTR (3′ _TR FXN) or a truncated 3′ UTR FXN, because the 3′ UTR FX or truncated 3′ UTR FXN does not include regulatory elements to modulate expression of FXN. Examples of 3′ UTRs include, but are not limited to SEQ ID NO: 40, SEQ ID NO: 41, SEQ ID NO 46, SEQ ID NO: 47, or a fragment thereof.

In some embodiments, the nucleic acid construct further includes a pair of inverted terminal repeats (ITR), wherein the nucleic acid construct is flanked on each said by an ITR. Exemplary ITR sequences include, but are not limited to SEQ ID NO: 38, SEQ ID NO: 39 and their reverse complements.

In some embodiments, the nucleic acid construct further includes a nucleic acid sequence encoding a polyadenylation (polyA) sequence, for example, a polyA bovine growth hormone sequence. SEQ ID NO: 63 is an exemplary sequence encoding a bovine growth hormone polyA sequence.

As used throughout, the term “nucleic acid” or “nucleotide” refers to deoxyribonucleic acids (DNA) or ribonucleic acids (RNA) and polymers thereof in either single- or double-stranded form, Sequences complementary to any of the sequences provided herein are also provided. It is understood that when an RNA is described, its corresponding cDNA is also described, wherein uridine is represented as thymidine. When a cDNA is described, its corresponding mRNA is also described. Unless specifically limited, the term encompasses nucleic acids containing known analogues of natural nucleotides that have similar properties as the reference nucleic acid and are metabolized in a manner similar to naturally occurring nucleotides. A nucleic acid sequence can include combinations of deoxyribonucleic acids and ribonucleic acids. Such deoxyribonucleic acids and ribonucleic acids include both naturally occurring molecules and synthetic analogues. The polynucleotides of the invention also encompass all forms of sequences including, but not limited to, single-stranded forms, double-stranded forms, hairpins, stein-and-loop structures, and the like.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al J. Biol. Chem. 260:2605-2608 (1985); and Rossolini et al., Mol. Cell. Probes 8:91-98 (1994)).

Provided herein are nucleic acid sequences including, consisting of or consisting essentially of a nucleic acid sequence having at least 60% identity to any one of SEQ ID NOs. 1-63. The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990). J. Mol. Biol. 215: 403-410 and Altschul f al (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et a, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T. and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat. Acad. Sci. USA 90:5873-5787 (1993)). One measure of similarity provided by the BLAST algorithm is the smallest sun probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10, and most preferably less than about 10⁻²⁰.

The recombinant nucleic acids provided herein can be included in expression cassettes for expression in a host cell or an organism of interest. The cassette may additionally contain at least one additional gene or genetic element to be cotransformed into the organism. Where additional genes or elements are included, the components are operably linked. The promoters of the Invention are capable of directing or driving expression of a coding sequence in a host cell. Other regulatory regions (i.e., transcriptional regulatory regions, and translational termination regions) can be included.

Additional regulatory signals include, but are not limited to, transcriptional initiation start sites, operators, activators, enhancers, other regulatory elements, ribosomal binding sites, an initiation codon, termination signals, and the like. See Sambrook et al. (1992) Molecular Cloning: A Laboratory Manual, ed. Maniatis et al. (Cold Spring Harbor Laboratory Press, Cold Spring Harbor, N.Y.) (hereinafter “Sambrook 11”); Davis et al., eds. (1980) Advanced Bacterial Genetics (Cold Spring Harbor Laboratory Press), Cold Spring Harbor, N.Y., and the references cited therein.

The expression cassette can also include a selectable marker gene for the selection of transformed cells. Marker genes include genes conferring antibiotic resistance, such as those conferring hygromycin resistance, kanamycin resistance, ampicillin resistance, gentamicin resistance, neomycin resistance, to name a few. Additional selectable markers are known and any can be used. Exemplary sequences for genes conferring ampicillin resistance and kanamycin resistance are provided herein as SEQ ID NO: 44 and SEQ ID NO: 45, respectively. The ampicillin resistance gene in any of the constructs described herein, for example, in pLP1001, pLP1002, pLP1003, pLp1004 or pLP1049, can be replaced with a kanamycin resistance gene.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

### Vectors

Also provided are vectors including any of the nucleic acid constructs described herein. In some embodiments, the vector is a plasmid. In some embodiments, the vector is a recombinant viral vector. In some embodiments, the vector is a DNA vector or PNA vector. Examples of viral vectors include, but are not limited to an adeno-associated virus (AAV) vector, a retroviral vector, a lentiviral vector, a herpes simplex viral vector, or an adenoviral vector. It is understood that any of the viral vectors described herein can be packaged into viral particles or virions for administration to the subject.

In some embodiments, the recombinant viral vector is an AV vector. In some embodiments, the viral vector is an AAV vector including a 5′ inverted terminal repeat and a 3′ inverted terminal repeat. In some embodiments, the AAV vector can be a single-stranded AAV vector or a self-complementary AAV vector.

As used herein, a “recombinant AAV vector” refers to an AAV vector including a nucleic acid sequence that is not normally present in AAV (i.e., a polynucleotide heterologous to AAV), for example, any of the nucleic acid constructs described herein, that expresses human frataxin. In general, the heterologous nucleic acid is flanked by at least one, and generally by two, AAV inverted terminal repeat sequences (ITRs). The term recombinant AAV vector encompasses both rAAV vector particles and recombinant AAV vector plasmids. A recombinant AAV vector may either be single-stranded (ssAAV) or self-complementary (scAAV).

In some embodiments, the recombinant AAV vector includes a nucleic acid sequence having at least 85% sequence identity to any one of SEQ ID NOs: 6-14, and 24-28. In some embodiments, the recombinant AAV vector includes any one of SEQ ID NOs: 6-14, and 24-28.

The recombinant AAV vector can further include viral sequences for packaging. Any missing viral functions can be supplied in trans by a packaging cell. For example, recombinant AAV vectors used in gene therapy may only possess inverted terminal repeat (ITR) sequences from the recombinant AAV genome and the balance of the vector can include sequences of interest (e.g., a 5′UTR FXN and a FXN nucleotide sequence). The ITR sequences can be included for packaging into AAV capsids. The packaging cell can also contain a plasmid that encodes other AAV genes (e.g., rep and cap), but lacks ITR sequences. The plasmid that encodes rep and cap genes may not be packaged in significant amounts due to a lack of ITR sequences. The packaging cell can also be infected with adenovirus as a helper virus, which can promote replication of the AAV vector and expression of A-AV genes from the plasmid that encodes rep and cap genes. The packaging cell can be transfected with a helper plasmid encoding gene products of helper viruses, such as adenovirus, which promotes replication of the AAV vector and expression of AAV genes from the plasmid that encodes rep and cap genes.

Purification of AAV particles from a packaging cell can involve growth of the packaging cells that produces the viral vectors, followed by collection of the viral vector particles from the cell supernatant and/or from the crude lysate. A-AV can then be purified, such as by ion exchange chromatography (e.g., U.S. Pat. Nos. 7,419,817 and 6,989,264), ion exchange chromatography and CsCl or iodixanol density centrifugation (e.g., PCT publication WO2011094198A10), immunoaffinity chromatography (e.g., WO2016128408) or purification using AVB Sepharose (e.g., GE Healthcare Life Sciences).

As used herein, a recombinant AAV particle or virion is a viral particle including at least one AAV capsid protein and an encapsidated recombinant AAV vector. As used herein, a recombinant AAV particle is a viral particle including at least one AAV capsid protein and an encapsidated recombinant AAV vector. An “.AAV virus,” AAV virion.” “AAV viral particle,” or “recombinant AAV vector particle” refers to a viral particle composed of at least one AAV capsid protein and an encapsidated polynucleotide recombinant AAV vector. If the particle includes a heterologous nucleic acid sequence (i.e. a nucleic acid sequence other than a wild-type AAV genome such as a transgene to be delivered to a mammalian cell), it can be referred to as a recombinant AAV vector. Thus, production of recombinant AAV particles or virion necessarily includes production of a recombinant AAV vector, as such a vector is contained within a recombinant AAV particle. Methods for producing AAV vectors and virions are known in the art. See, for example, Shin et al. “Recombinant Adeno-Associated Viral Vector Production and Purification,” Methods Mol. Biol. 798: 267-284 (2012)).

Also provided is a cell including any of the vectors described herein. The host cell can be an in vitro, ex vivo, or in vivo host cell. Populations of any of the host cells described herein are also provided. A cell culture including one or more host cells described herein is also provided. Methods for the culture and production of many cells, including cells of bacterial (for example E. coli and other bacterial strains), animal (especially mammalian), and archaebacterial origin are available in the art. See e.g., Sambrook. Ausubel, and Berger (all supra), as well as Freshney (1994) Culture of Animal Cells, a Manual of Basic Technique, 3^{rd} Ed, Wiley-Liss, New York and the references cited therein; Doyle and Griffiths (1997) Mammalian Cell Culture: Essential Techniques John Wiley and Sons, NY; Humason (1979) Animal Tissue Techniques, 4^{th} Ed. W. H. Freeman and Company; and Ricciardelli, et al., (1989) In vitro Cell Dev. Biol. 25:1016-1024.

The host cell can be a prokaryotic cell, including, for example, a bacterial cell. Alternatively, the cell can be a eukaryotic cell, for example, a mammalian cell. In some embodiments, the cell can be an HEK293T cell, a Chinese hamster ovary (CHO) cell, a COS-7 cell, a HELA cell, an avian cell, a myeloma cell, a Pichia cell, an insect cell or a plant cell. A number of other suitable host cell lines have been developed and include myeloma cell lines, fibroblast cell lines, and a variety of tumor cell lines such as melanoma cell lines. The vectors containing the nucleic acid segments of interest can be transferred or introduced into the host cell by well-known methods, which vary depending on the type of cellular host.

Methods for introducing vectors into cells are known in the art. As used herein, the phrase “introducing” in the context of introducing a nucleic acid into a cell refers to the translocation of the nucleic acid sequence from outside a cell to inside the cell. In some cases, introducing refers to translocation of the nucleic acid from outside the cell to inside the nucleus of the cell. Various methods of such translocation are contemplated, including but not limited to, electroporation, nanoparticle delivery, viral delivery, contact with nanowires or nanotubes, receptor mediated internalization, translocation via cell penetrating peptides, liposome mediated translocation. DEAE dextran, lipofectamine, calcium phosphate or any method now known or identified in the future for introduction of nucleic acids into prokaryotic or eukaryotic cellular hosts. A targeted nuclease system (e.g., an RNA-guided nuclease (for example, a CRISPR/Cas9 system), a transcription activator-like effector nuclease (TALEN), a zinc finger nuclease (ZFN), or a megaTAL (MT) (Li et al. Signal Transduction and Targeted Therapy 5. Article No. 1 (2020)) can also be used to introduce a nucleic acid into a host cell.

### AAV Serotypes

Recombinant AAV particles including the recombinant AAV vectors provided herein can include or be derived from any natural or recombinant AAV serotype. The AAV particles can be, or can be based on, a serotype selected from any of the following serotypes, and variants thereof including, but not limited to, AAV1, AAV10, AAV106.1/hu.37, AAV11, AAV114.3/hu.40, AAV12, AAV127.2/hu.41, AAV127.5/hu.42, AAV128.1/hu.43, AAV128.3/hu.44, AAV130.4/hu.48, AAV145.1/hu.53. AAV145.5/hu.54, AAV145.6/hu.55, AAV16.12/hu.11, AAV16.3, AAV16.8/hu.10, AAV161.10/hu.60. AAV161.61/hu.61, AAV1-7/rh.48, AAV1-8/rh.49, AAV2, AAV2.5T, AAV2-15rh.62, AAV223.1, AAV223.2. AAV223.4, AA V223.5. AAV223.6, AAV223.7, AAV2-3/rh.61, AAV24.1, AAV2-4/rh.50, AAV2-5/rh.51, AAV27.3, AAV29.3/bb.1, AAV29.5,bb.2, AAV2G9, AAV-2-pre-miRNA-101, AAV3, AAV3.1/hu.6, AAV3.1/hu.9, AAV3-11/rh.53. AAV3-3, AAV33.12/hu.17, AAV33.4hu.15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-lb AAV42-2, AAV42-3a. AAV42-3b, AAV42-4, AAV42-5a, AAV42-5b, AAV42-6b, AAV42-8, AAV42-aa AAV43-1, AAV43-12, AAV43-20, AAV43-21, AAV43-23, AAV43-25, AAV43-5, AAV4-4, AAV44.1, AAV44.2, AAV44.5. AAV46.2/hu.28, AAV46.6/hu.29, AAV4-8/r11.64, AAV4-8/rh.64, AAV4-9/rh.54, AAV5. AAV52.1/hu.20, AAV52hu.19. AAV5-22/rh.58, AAV5-3/rh.57, AAV54.1 hu.21, AAV54.2/hu.22, AAV54.4R/hu.27. AAV54.5/hu.23, AAV54.7/hu.24, AAV58.2/hu.25, AAV6, AAV6.1, AAV6.1L2. AA-V6.2 AAV7, AAV7.2, AAV7,3/hu 0.7, AAV8, AAV-8b. AAV-8h, AAV9, AAV9,11. AAV9.13, AAV9.16, AAV9.24, AAV9.45, AAV9.47, AAV9.61, AAV9.68, AAV9.84, AAV9.9, AAVA3.3, AAVA3.4, AAVA3.5, AAVA3.7, AAV-b. AAVC1, AAVC2 AAVC5, AAVCh.5, AAVCh.5R1, AAVcy.2, AAVey.3, AAVey.4, AAVey.5, AAVCy.5R1, AAVCy.5R2, AAVCy.5R3. AAVCy.5R4, AAVcy.6. AAV-DJ, AAV-DJ8S AAVF3, AAVF5, AAV-h, AAVH-1/hu.1, AAVH2, AAVH-5<hu.3, AAVH6, AAVhE11, AAVhER1.14. AAVhEr1.16, AAVhEr1.18, AAVhER1.23, AAVhEr135, AAVhEr1.36, AAVEr1.5 AAVhEr1.7, AAEVhEr1.8, AkVhEr2.16, AAVhrEr2.29, AAVhEr2.30, AAVhEr231, AAVEr2.36, AAVhEr2.4. AAVhEr31. AAVhu.1, AAVhu.10, AAVhu.11, AAVhu.11, AAVhu.12. AAVhu.13, AAVhu.14/9, AAVhu.15, AAVhu.16, AAVhu.17, AAVhu.18, AAVhu.19, AAVhu.2, AAVhu.20, AAVhu.21. AAVhu.22, AAVhu.23.2, AAVhu.24, AAVhu.25, AAVhu.27, AAVhu.28. AAVhu.29, AAVhu.29R, AAVhu.3, AA-hu.31. AAVhu.32, AAVhu.34, AAVhu.35, AAVhu.37, AAVhu.39, AAVhu.4, AAVhu.40. AAVhu.41, AAVhu.42, AAVhu.43, AAVhu.44. AAVhu.44R1, AAVhu.44R2, AAVhu.44R3, AAVhu.45, AAVhu.46, AAVhu.47; AAVhu.48, AAVhu.48R1, AAVhu.48R2, AAVhu.48R3, AAVhu.49. AAVhu.5. AAVhu.51, AAVhu.52, AAVhu.53, AAVhu.54. AAVhu.55, AAVhu.56, AAVhu.57, AAVhu.58, AAVhu.6 AAVhu.60, AAVhu.61, AAVhu.63, AAVhu.64, AAVhu.66, AAVhu.67, AAVhu.7, AAVhu.8, AAVhu.9, AAVhu.t 19, AAVLG-10/rh.40, AAVLG-4/rh.38, AAVLG-9,hu.39, AAVLG-9,hu.39, AAV-LK01, AAV-LK02, AAVLK03, AAV-LK03, AAV-LK04, AAV-LK05, AAV-LK06, AAV-LK07, AAV-LK08, AAV-LK09, AAV-LK10. AAV-LK11, AAV-LK12, AAV-LK13, AAV-LK14,AAV-LK15,AAV-LK17,AAV-LK18,AAV-LK19 AAVN721-8/rh.43, AAV-PAEC, AAV-PAEC11. AAV-PAEC12, AAV-PAEC2. AAV-PAEC4, AAV-PAEC6, AAV-PAEC7, AAV-PAEC8, AAVpi.1, AAVpi.2, AAVpi.3, AAVrh.10, AAVrh.12. AAVrh.13, AAVrh.13R, AAVrh.14, AAVrh.17, AAVrh.18, AAVrh.19. AAVrh.2, AAVrh.20, AAVrh.21. AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2. AAVrh.38, AAVrh.39, AAVrh.40, AAVrh.43, AAVrh.44, AAVrh.45, AAVrh.46, AAVrh.47, AAVrh.48. AAVrh.48, AAVrh.48.1, AAVrh48.1.2, AAVrh.48.2, AAVrh.49, AAVrh0.50, AAVrh.51, AAVrh.52. AAVrh.53, AAVrh.54, AAVrh.55. AAVrh.56, AAVrh.57, AAVrh.58, AAVrh.59. AAVrh.60, AAVrh.61, AAVrh.62, AAVrh64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8. AAVrh.8R, AAVrh8R, AAVrh8R A586R mutant, AAVrh8R R533A mutant, BAAV, BNP61 AAV, BNP62 AAV, BNP63 AA-V, bovine AAV, caprine AAV. Japanese AAV 10, true type AAV (ttAAV), UPENN AAV 10. AAV-LK16, AAAV, AAV Shuffle 100-1, ALAY Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, and/or AAA SM 10-8.

The AAV serotype can be, or have, a mutation in the AAV9 sequence, as described by N Pulicherla et al. (Molecular Therapy 19(6):1070-1078 (2011), such as, but not limited to, AAV9.9, AAV9.11, AAV9.13, AAV9.16, AAV9.24, AAV9.45, AAV9.47, AAV9.61, AAV9.68, AAV9.84.

The AAV serotype can be, or have, a sequence as described in United States Pat. No. US6156303, such as, but not limited to, AAV3B (SEQ ID NO: 1 and 10 of U.S. Pat. No. 6,156,303). AAV6 (SEQ ID NO: 2, 7 and 11 of U.S. Pat. No. 6,156,303) AAV2 (SEQ ID NO: 3 and 8 of U.S. Pat. No. 6,156,303), AAV3A (SEQ ID NO: 4 and 9, of US6156303), or derivatives thereof.

The serotype can be AAVDJ or a variant thereof such as AAVDJ8 (or AAV-DJ8), as described by Grinmm et al. (Journal of Virology 82(12): 5887-5911 (2008)). The amino acid sequence of AAVDJ8 can include two or more mutations in order to remove the heparin binding domain (HBD). The AAV-DJ sequence described as SEQ ID NO: 1 in U.S. Pat. No. 7,588,772, can include two mutations: (1) R587Q where arginine (R; Arg) at amino acid 587 is changed to glutamine (Q Gin) and (2) R590T where arginine (R; Arg) at amino acid 590 is changed to threonine (T; Thr). As another non-limiting example, the amino acid sequence of AAVDJ8 can include three mutations: (1) K406R where lysine (K; Lys) at amino acid 406 is changed to arginine (R; Arg), (2) R587Q where arginine (R; Arg) at amino acid 587 is changed to glutamine (Q: Gin) and (3) R590T where arginine (R; Arg) at amino acid 590 is changed to threonine (T; Thr).

The AAV serotype can be, or have, a sequence as described in International Publication No. WO2015121501, such as, but not limited to, true type AAV (ttAAV) (SEQ ID NO: 2 of WO2015121501). “UPenn AAV10” (SEQ ID NO: 8 of WO2015121501), “Japanese AAV10” (SEQ ID NO: 9 of WO2015121501), or variants thereof.

AAV capsid serotype selection or use can be from a variety of species. For example, the AAV can be an avian AAV (AAAV). The AAAV serotype can be, or have, a sequence as described in U.S. Pat. No. 9,238,800, such as, but not limited to, AAAV (SEQ ID NO: 1, 2, 4, 6, 8& 10, 12, and 14 of US9238800), or variants thereof.

The AAV can be a bovine AAV (BAAV). The BAAV serotype can be, or have, a sequence as described in U.S. Pat. No. 9,193,769, such as, but not limited to, BAAV (SEQ ID NO: 1 and 6 of U.S. Pat. No. 9,193,769), or variants thereof. The BAAV serotype can be, or have, a sequence as described in U.S. Pat. No. 7,427,396, such as, but not limited to, BAAV (SEQ ID NO: 5 and 6 of U.S. Pat. No. 7,427,396), or variants thereof.

The AAV can be a caprine AAV. The caprine AAV serotype can be, or have, a sequence as described in U.S. Pat. No. 7,427,396, such as, but not limited to, caprine AAV (SEQ ID NO: 3 of US7427396), or variants thereof.

The AAV can be engineered as a hybrid AAV from two or more parental serotypes. The AAV can be AAV2G9 which includes sequences from AAV2 and AAV9. The AAV2G9 AAV serotype can be, or have, a sequence as described in U.S. Patent Publication No. US20160017005.

The AAV can be a serotype generated by the AAV9 capsid library with mutations in amino acids 390-627 (VP1 numbering) as described by Pulicherla et al. (Molecular Therapy 19(6):1070-1078 (2011). The serotype and corresponding nucleotide and amino acid substitutions can be, but is not limited to, AAV9.1 (G1594C; D532H), AAV6.2 (T1418A and T1436X; V473D and 1479K), AAV9.3 (T1238A; F413Y), AAV9.4 (T1250C and A1617T; F417S), AAV9.5 (A1235G. A1314T, A1642G, C1760T; Q412R. T548A, A587V), AAV9.6 (T1231A; F411I), AAV9.9 (G1203A, G1785T; W595C). AAV9.10 (A1500G. T1676C; M559T), AAV9.11 (A1425T, A1702C, A1769T; T568P, Q590L), AAV9.13 (A1369C, A1720T; N457H. T574S), AAV9.14 (T1340A, T1362C, T1560C. G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V606I) AAV9.40 (A1694T, E565V), AAV9.41 (A1348T. T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 947 (G1241A, G1358A, A1669G, C1745T; S414N, G453D. K557E, T582I), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q. S469R, A555V, G604V), AAV9.54 (C1531A; T1609A; L511 I, L537M) AAV9.55 (T1605A; F535L), AAV9.58 (C1475T, C1579A; T492I, H527N, AAV.59 (T1336C; Y446H) AAV9.61 (A1493T; N498I), AAV9.64 (C1531A, A1617T; L5111), AAV9.65 (C1335T. T1530C, C1568A: A523D), AAV9.68 (C1510A; P504T) AAV9.80 (G1441A;G4S1R), AAV9.83 (C1402A, A1500T; P468T, E500D), AAV9.87 (T1464C, T1468C: S490P), AAV9.90 (A1196T: Y399F), AAV9.91 (T1316G. A1583T, C1782G, T1806C; L439R, K5281), AAV9.93 (A1273G, A1421G. A1638C. C1712T, G1732A, A1744T, A1832T: S425G, Q474R, Q546H, P571L, G578R, T582S, D61 IV) AAV9.94 (A1675T; M559L) and AAV9.95 (T1605A; F535L).

The AAV can be a serotype including at least one AAV capsid CD8+ T-cell epitope. As a non-limiting example, the serotype can be AAV1, AAV2 or AAV8.

The AAV can be a variant, such as PHP.A or PHP.B as described in Deverman. 2016. Nature Biotechnology. 34(2): 204-209.

The present disclosure also provides a method of generating a packaging cell that includes creating a cell line that stably expresses all of the necessary components for AAV particle production. For example, a plasmid (or multiple plasmids) including a recombinant AAV genome lacking AAV rep and cap genes, AAV rep and cap genes separate from the recombinant AAV genome, and a selectable marker, such as a neomycin resistance gene, are integrated into the genome of a cell. AAV genomes have been introduced into bacterial plasmids by procedures such as GC tailing (Samulski et al., 1982, Proc. Natl, Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line can then be infected with a helper virus, such as adenovirus. Some advantages of this method are that the cells are selectable and are suitable for large-scale production of recombinant AAV. Other examples of suitable methods employ adenovirus or baculovirus, rather than plasmids, to introduce recombinant AAV genomes and/or rep and cap genes into packaging cells. General principles of recombinant AAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol, Cell. Biol. 4:2072 (1984); Hermonat et al., Proc. Natl. Acad. Sci. USA. 81:6466 (1984); Tratschin et al., Mol. Cell. Biol. 5:3251 (1985); McLaughlin et al., J. Virol., 62:1963 (1988); and Lebkowski et al. 1988 Mol. Cell. Biol., 7:349 (1988). Samulski et cl. (1989, J. Virol., 633822-3828): U.S. Pat. No. 5,173,414: WO 95/13365 and corresponding U.S. Pat. No. 5,658,776; WO 95/13392; WO 96/17947; PCT/US98/18600; WO 97/09441 (PCT/US96.4423); WO 97/08298 (PCT/US96/13872); WO 97/21825 (PCT/US96/20777): WO 97/06243 (PCT/FR96/01064); WO99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595.

AAV vector serotypes can be matched to target cell types. For example, the following exemplary cell types can be transduced by the indicated AAV serotypes among others. See Table 1.

TABLE 1
Tissue/Cell Types and Serotypes

| Tissue/Cell Type | Serotype |
|---|---|
| Liver | AAV3, AA5, AAV8, AAV9 |
| Skeletal muscle | AAV1, AAV7, AAV6, AAV8, |
| Central nervous system | AAV1, AAV4, AAV5, AAV8, |
| RPE | AAV5, AAV4, AAV2, AAV8, |
| | AAV9 |
| Photoreceptor cells | AAV5, AA8, AAV9, AAVrh8R |
| Lung | AAV9, AAV5 |
| Heart | AAV8 |
| Pancreas | AAV8 |
| Kidney | AAV2, AAV8 |

### Pharmaceutical Compositions

Provided herein is a pharmaceutical composition including any of the recombinant viral vectors or viral particles described herein. The pharmaceutical compositions can include additional components suitable to, for example, increase delivery (e.g., increase infection of targeted cells and/or increase the range of cells that can be infected), increase stability of the recombinant vector, or decrease immunogenicity of the recombinant vector, for example, an AAV vector. For example, the pharmaceutical compositions can include a pharmaceutically acceptable carrier, excipient, and/or sail. The pharmaceutically acceptable carrier can exclude buffers, compounds, cryopreservation agents, preservatives, or other agents in amounts that can substantially interfere with the delivery or activity of the recombinant AAV vector to a patient. Exemplary liquid carriers are sterile aqueous solutions that contain no materials in addition to the recombinant AAV vector and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Examples of such additional liquid phases are glycerin, vegetable oils such as cottonseed oil, and water-oil emulsions.

The pharmaceutical compositions can be delivered to a subject, so as to allow production of an expression product in the cell(s) of the subject. Pharmaceutical compositions include sufficient genetic material that allows the recipient to produce an effective amount of an expression product that modulates FXN expression in a cell and/or treats FA in a subject.

In some embodiments, the pharmaceutical compositions also contain a pharmaceutically acceptable excipient. Such excipients include any pharmaceutical agent that does not itself induce an immune response harmful to the individual receiving the composition, and which may be administered without undue toxicity. Pharmaceutically acceptable excipients include, but are not limited to, liquids such as water, saline, glycerol, sugars and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying, agents, pH buffering substances, and the like, may be present in such vehicles. The preparation of pharmaceutically acceptable carriers, excipients and formulations containing these materials is described in, e.g., Remington: The Science and Practice of Pharmacy, 22nd edition, Loyd V. Allen et al. editors, Pharmaceutical Press (2012).

Pharmaceutical formulations suitable for parenteral administration may be formulated in aqueous solutions, preferably in physiologically compatible buffers such as Hanks's solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

### Genetically Modified Cells

Also provided herein are genetically modified cells including any of the nucleic acid constructs or recombinant viral vectors described herein. As used herein, a “genetically modified cell” refers to a cell that has at least one genomic modification as a result of introducing any of the nucleic acid constructs or recombinant viral vectors described herein, into the cell. The genetically modified cells can be in vitro, ex viva or in vivo genetically modified cells.

The genetically modified cells can be any suitable genetically modified cell, such as those selected from the group consisting of a human stem cell (for example, a multipotent stem cells, e.g., a mesenchymal stem cell that can differentiate into neurons and cardiomyocytes), human neuron, a human cardiomyocyte, a human smooth muscle myocyte, a human skeletal myocyte, and a human hepatocyte.

In some embodiments, bone-marrow derived mesenchymal stem cells are isolated from a subject having FA, and genetically modified to insert a nucleic acid construct including a 5′UTR and a nucleic acid sequence encoding human FX. The genetically modified cells are then autologously transplanted back into the subject. In some embodiments, the genetically modified cells can be systemically delivered to allow targeted delivery of the grafts to the brain and heart of FA patients. See, for example, Tajiri et al. “Autologous Stem Cell Transplant with Gene Therapy for Friedreich Ataxia” Med. Hypotheses 83(3): 296-298 (2014). Methods for introduction of nucleic acids and vectors for genetic modification of cells are described above.

The term “genetic modification” refers to any change in the DNA genome (or RNA genome in some cases) of a cell, organism, virus, viral vector, or other biological agent. Non-limiting examples of genetic modifications include an insertion, a deletion, a substitution, a procedure such as a transfection or transformation where exogenous nucleic acid is added to a cell and/or organism, and cloning techniques.

The term “insertion” refers to an addition of one or more nucleotides in a nucleic acid sequence. Insertions can range from small insertions of a few nucleotides to insertions of large segments such as a cDNA or a gene.

The term “deletion” refers to a loss or removal of one or more nucleotides in a nucleic acid sequence or a loss or removal of the function of a gene. In some cases, a deletion can include, for example, a loss of a few nucleotides, an exon, an intron, a gene segment, or the entire sequence of a gene. In some cases, deletion of a gene refers to the elimination or reduction of the function or expression of a gene or its gene product. This can result from not only a deletion of sequences within or near the gene, but also other events (e.g., insertion, nonsense mutation) that disrupt the expression of the gene.

The term “substitution” refers to a replacement of one or more nucleotides in a nucleic acid sequence with an equal number of nucleotides.

Genetic modification of a nucleic acid sequence can result in a “recombinant” sequence. For example, the present disclosure provides “recombinant AAV vectors,” which have been genetically modified to include elements disclosed herein.

### Methods of Treatment

Also provided are methods for treating FA. The methods include administering to a subject having FA, a therapeutically effective amount of any of the recombinant AAV particles provided herein.

As used throughout, by subject is meant an individual. The subject can be an adult subject or a pediatric subject. Pediatric subjects include subjects ranging in age from birth to eighteen years of age. Preferably, the subject is an animal, for example, a mammal such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical formulations are contemplated herein.

A used throughout “treat,” “treating,” and “treatment” refer to a method of reducing or delaying one or more effects or symptoms of FA. The subject can be diagnosed with FA. Treatment can also refer to a method of reducing the underlying pathology rather than just the symptoms. The effect of the administration to the subject can have the effect of, but is not limited to, reducing one or more symptoms of the disease, a reduction in the severity of the disease, the complete ablation of the disease, or a delay in the onset or worsening of one or more symptoms. For example, a disclosed method is considered to be a treatment if there is about a 10% reduction in one or more symptoms of the disease (e.g., muscle loss, ataxia in anus and legs in a subject, diabetes, cardiomyopathy, etc.) when compared to the subject prior to treatment or when compared to a control subject or control value. Thus, the reduction can be about a 10, 20, 30, 40, 50, 60, 70, 80, 90, 100%, or any amount of reduction in between.

Also provided are methods of modulating expression of FXN in a human cell. In some embodiments, the methods include introducing into the human cell, any of the recombinant AAV vectors provided herein. In some embodiments the cell is in a subject.

Also provided are methods for increasing adenosine triphosphate (ATP) concentration in a human cell of a subject with FA. The methods include administering to the subject a therapeutically effective amount of any of the recombinant AAV particles provided herein. In some methods, the human cell is selected from the group consisting of a neuron, a cardiomyocyte, a smooth muscle myocyte, a skeletal myocyte, and a hepatocyte.

Also provided are methods for increasing ATP concentration in a human cell of a subject with FA. The methods include administering a, therapeutically effective amount of any of the recombinant AAV particles provided herein. In some methods, the human cell is selected from the group consisting of: a neuron, a cardiomyocyte, a smooth muscle myocyte, a skeletal myocyte, and a hepatocyte.

As used herein, an increase can be an increase of about 5%, 10%, 20%, 30%, 40%, 50%, 60%, 70%, 80%, 90%, 100%, 200%, 300%, 400% or greater. Increases in the levels of ATP expressed in cells of FA patients can be beneficial for ameliorating one or more symptoms of the disease, increasing long-term survival, and/or reducing side effects associated with other treatments. Upon administration of the recombinant AAV vectors disclosed herein to human FA patients, the recombinant AAV vectors can express increased, yet modulated, levels of FXN, and ATP production by mitochondria can be increased, relative to the disease state. 100,000 to 500,000 cells: 500,000 to 1,000,000 cells; 1,000.000 cells to 2,500,000 cells; 2,500,000 to 5,000,000 cells; 5,000,000 to 10,000.000 cells: 10,000,000 to 50,000,000 cells; 50,000,000 to 100,000,000; 100,000,000 to 250,000,000 cells: 150,000,000 to 300,00,000 cells: 250,000,000 to 500,000,000 cells; 500,000,000 to 1,000,000,000 cells: 1,000,000.000 to 5,000,00,000 cells; 5,000,000,000 to 10,000,000,000 cells; 10,000,000,000 to 20,000,00000 cells: 15,000,000,000 to 30,000,000,000 cells; 30,000,000,000 to 50,000,000,000 cells: 50,000,000,000 to 75,000,000,000 cells: or 75,000,000,000 to 100,00000,000 cells in FA patients to whom such recombinant AAV vectors are administered can express increased, yet modulated levels of FXN, and ATP production by mitochondria can be increased, relative to the disease state.

The term “effective amount,” as used throughout, is defined as any amount necessary to produce a desired physiologic response, for example, reducing or delaying one or more effects or symptoms of FA. Effective amounts and schedules for administering the recombinant AAV virions described herein can be determined empirically and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, unwanted cell death, and the like. Generally, the dosage will vary with the species, age, body weight, general health, sex and diet of the subject, the mode and time of administration and severity of the particular condition and can be determined by one of skill in the art. The dosage can be adjusted by the individual physician in the event of any contraindications. Dosages can vary and can be administered in one or more doses.

An effective amount of any of the recombinant AAV virions described herein will vary and can be determined by one of skill in the art through experimentation and/or clinical trials. For example, for in vivo injection, for example, injection directly into the inner ear of a subject, an effective dose can be from about 10⁶ to about 10¹⁵ recombinant rAAV virions, or any values in between this range, for example, about 10⁶, 10⁷, 10⁸, 10⁹. 10¹⁰, 10¹¹, 10¹², 10¹³ 10¹⁴, or 10¹⁵ recombinant AAV particles.

In some embodiments, the number of rAAV particles administered to a subject may be on the order ranging from about 10⁶ to 10¹⁵ vector genomes (vgs)/ml, such as for example, about 10⁶, 10⁷, 10⁸. 10⁹ 10¹⁰, 10¹¹, 10¹², 10¹³, 10¹⁴ or 10¹⁵ vg/ml. In some embodiments, the number of rAAV particles administered to a subject can be from about 10⁶ to 10¹⁵ vg/kg, or any values in between these amounts, such as for example, about 10⁶, 10⁷, 10⁸, 10⁹, 10¹⁰, 10¹¹, 10¹², 10¹³, 10¹⁴, or 10¹⁵ vg/kg. Other effective dosages can be readily established by one of ordinary skill in the art through routine trials establishing dose response curves.

Any of the methods provided herein can further include administering a second therapeutic agent to the subject having FA, for example, a beta blocker, an ACE inhibitor, an antioxidant, a diuretic, an anti-diabetic agent, or a combination thereof.

The compositions described herein are administered in a number of ways depending on whether local or systemic treatment is desired. The compositions are administered via any of several routes of administration, intraparenchymal injection, intravenously, intrathecally, intramuscularly, intracistemally, intracoronary injection, intramyocardium injection, intradermally, endomyocardiac injection, or a combination thereof. In some embodiments, the compositions are administered canalostomy into the posterior semicircular canal of the subject. Effective doses for any of the administration methods described herein can be extrapolated from dose-response curves derived from in vitro or animal model test systems.

### General Terminology

The grammatical articles “a”, “an”, and“the”, as used herein, are intended to include “at least one” or “one or more”, unless otherwise indicated, even if “at least one” or “one or more” is expressly used in certain instances. Thus, the articles are used herein to refer to one or more than one (i.e., to “at least one”) of the grammatical objects of the article. Further, the use of a singular noun includes the plural, and the use of a plural noun includes the singular, unless the context of the usage requires otherwise.

The use herein of the terms “including.” “including,” or “having.” and variations thereof, is meant to encompass the elements listed thereafter and equivalents thereof as well as additional elements. Embodiments recited as “including,” “including,” or “having” certain elements are also contemplated as “consisting essentially of and “consisting of those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re, Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “including.”

Any numerical range recited in this specification describes all sub-ranges of the same numerical precision (i.e., having the same number of specified digits) subsumed within the recited range. For example, a recited range of “1.0 to 10.0” describes all sub-ranges between (and including) the recited minimum value of 1.0 and the recited maximum value of 10.0, such as, for example, “2.4 to 7.6,” even if the range of “²0.4 to 7.6” is not expressly recited in the text of the specification. Also, unless expressly specified or otherwise required by context, all numerical parameters described in this specification (such as those expressing values, ranges, amounts, percentages, and the like) may be read as if prefaced by the word “about.” even if the word “about” does not expressly appear before a number. “About” is used to provide flexibility to a numerical range endpoint by providing that a given value may be “slightly above” or “slightly below” the endpoint without affecting the desired result.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

The present disclosure will be more fully understood by reference to the following examples, which provide illustrative, non-limiting aspects of the invention.

As set forth above, Friedreich's ataxia (FA) is a rare mitochondrial disorder characterized by ataxia, cardiomyopathy, and diabetes. Currently there is no cure for this disease. In the context of developing an AAV-based approach, the complexity of designing a therapeutic cassette encoding frataxin (FXN), along with identification of a capsid targeting vulnerable cell types, has remained elusive.

The following examples describe compositions and methods for treatment of FA. FA can be treated by modulating expression of the FXN gene via, for example, a viral vector which promotes increased, yet modulated, FXN expression in cells homozygous for GAA trinucleotide repeat alleles. The FXN nucleotide sequence can be operably linked to a 5′ UTR FXN, which can modulate FXN expression. Modulated FXN expression is desired to achieve modulated physiological levels of FXN expression and avoid elevated levels of FXN expression. The non-modulated, elevated physiological levels of FXN can result in reduced mitochondrial respiration, which leads to mitochondrial toxicity. The described compositions and methods represent a novel strategy for treatment of FA, as described and illustrated herein.

### Example 1-Effects of the 5′ UTR FXN Sequence on FXN Gene Expression

An experiment was conducted to determine whether expression of a human FXN nucleotide sequence could be affected by inclusion of a 5′ UTR FX sequence.

Four versions of a plasmid vector encoding a recombinant AAV vector genome were constructed. A first version (SEQ ID NO: 7) includes a codon-optimized human FXN nucleotide sequence (SEQ ID NO: I) operably linked to a desmin promoter sequence (SEQ ID NO: 4) and further includes a desmin 5′UTR (SEQ ID NO: 22) operably positioned between the desmin promoter sequence and the human FXN nucleotide sequence. A second version (SEQ ID NO: 8) includes the codon-optimized human FXN nucleotide sequence operably linked to the desmin promoter sequence (SEQ ID NO: 4) and further includes a desmin 5′UTR (SEQ ID NO: 22) and 5′UTR FXN sequence (SEQ ID NO: 2) operably positioned between the desmin promoter sequence and the human FXN nucleotide sequence. A third version (SEQ ID NO: 9) is similar to the first version, except that the third version further includes a C-terminal V5 epitope tag in-frame with the human FXN nucleotide sequence. A fourth version (SEQ ID NO: 10) is similar to the second version, except that the fourth version further includes a C-terminal V5 epitope tag in-frame with the human FXN nucleotide sequence.

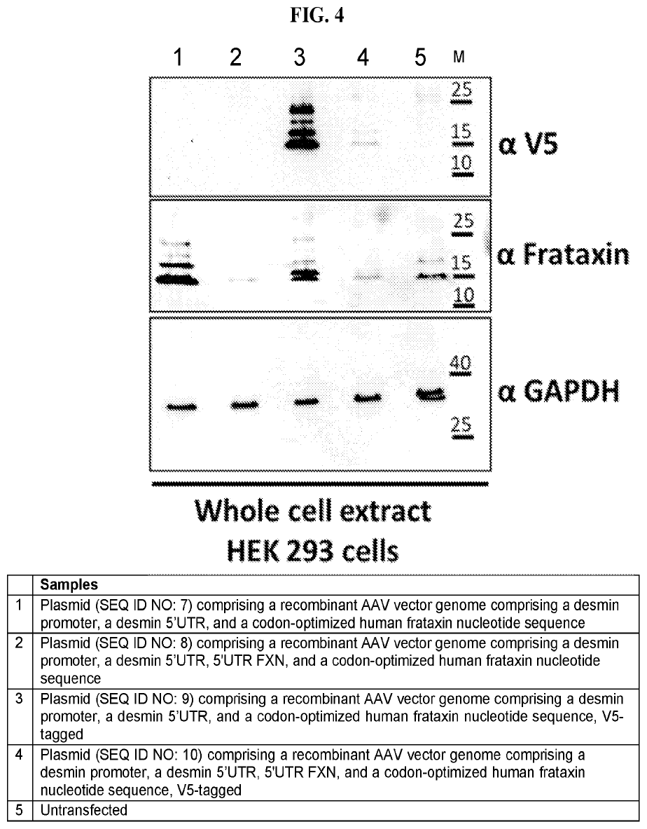

After confining the accurate construction of the four plasmid vector versions via Sanger sequencing, each of the four versions were transfected into separate HEK 293 cell populations, using commercially available transfection reagents according to the manufacturer's instructions. 48 hours post-transfection the four cell populations were collected and total protein was harvested in RIPA buffer. Samples of the total protein (whole cell extract) were subjected to SDS-PAGE and immunoblotting. As indicated in FIG. 4, blots were probed using commercially available primary antibodies against the V5 epitope (α V5), human frataxin (α Frataxin), and, as a loading control. GAPDH (glyceraldehyde 3-phosphate dehydrogenase) (α GAPDH). Subsequently, blots were probed with HRP-conjugated secondary antibodies.

Lane 1 shows results for HEK 293 cells transfected with the first version of the plasmid, including the human FXN nucleotide sequence without a 5′ UTR FXN. No signal was visible on the anti-V5 blot, since no V5 epitope is included in the first plasmid version. Additionally, a signal was visible on the anti-frataxin blot.

Lane 2 shows results for HEK 293 cells transfected with the second version of the plasmid, including the human FXN nucleotide sequence with a 5′ UTR FXN. No signal was visible on the anti-V5 blot, since no V5 epitope is included in the second plasmid version.

Additionally, a signal was visible on the anti-frataxin blot. The anti-frataxin signal in lane 2 was less intense than the signal in lane 1.

Lane 3 shows results for HEK 293 cells transfected with the third version of the plasmid, including the human FXN nucleotide sequence without a 5′ UTR FXN and with a V5 epitope tag. Signal was visible on the anti-V5 blot, and a signal was visible on the anti-frataxin blot.

Lane 4 shows results for HEK 293 cells transfected with the fourth version of the plasmid, including the human FXN nucleotide sequence with a 5′ UTR FXN and with a V5 epitope tag. Signal was visible on the anti-V5 blot, and a signal was visible on the anti-frataxin blot. The anti-frataxin signal and anti-V5 signal in lane 4 were less intense than the corresponding signals in lane 3.

Lane 5 shows results for untransfected HEK 293 cells. Signal from endogenous frataxin and GAPDH is visible on the anti-frataxin and anti-GAPDH blots, respectively.

These data provide evidence that expression of a human FXN nucleotide sequence can be modulated by inclusion of a 5′ UTR FXN sequence. Surprisingly, the 5′ UTR FXN sequence was found to decrease expression of the frataxin protein encoded by the plasmid construct, relative to a construct without the 5′ UTR sequence.

### Example 2—Effects of the 5′ UTR FXN Sequence on FXN Gene Expression

An experiment was conducted to further investigate how expression of a human FXN nucleotide sequence in neuron-derived cells can be affected by inclusion of a 5′ UTR FXN sequence.

As in Example 1, separate cell populations were transfected with each of the four plasmid versions. In the instant Example, SK-N-SH cells (a neuroblastoma cell line) were used, instead of the HEK 293 cells of Example 1. Transfections and immunoblotting were performed as described in Example 1.

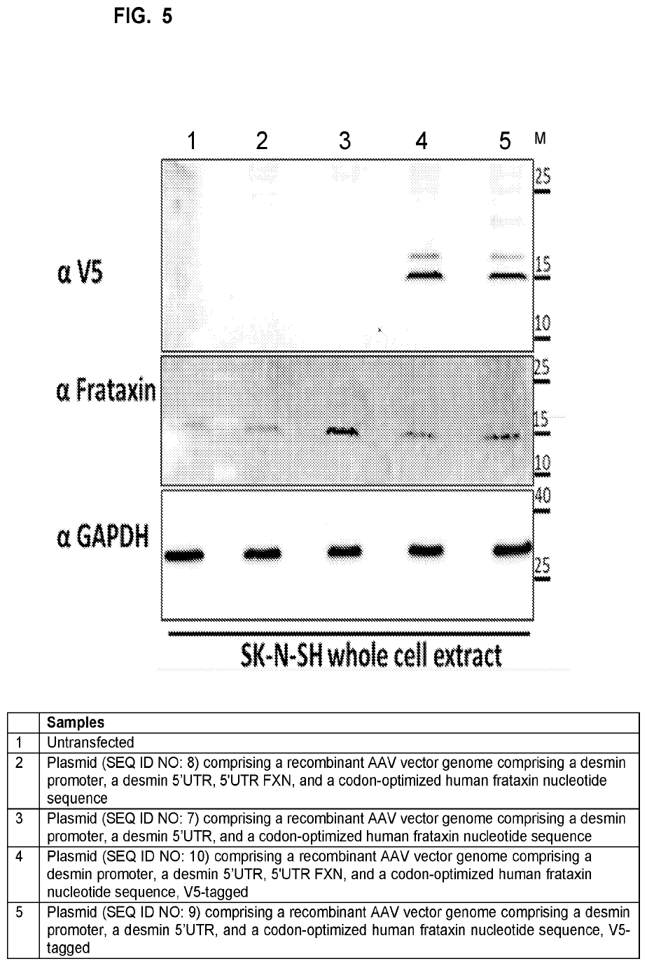

Referring to FIG. 5, lane 1 shows immunoblotting results for untransfected SK-N-SH cells. Signal from endogenous frataxin and GAPDH is visible on the anti-frataxin and anti-GAPDH blots, respectively.

Lane 2 shows results for SK-N-SH cells transfected with the second version of the plasmid, including the human FXN nucleotide sequence with a 5′ UTR FXN. No signal was visible on the anti-V5 blot, since no V5 epitope is included in the second plasmid version. Additionally, a signal was visible on the anti-frataxin blot.

Lane 3 shows results for SK-N-SH cells transfected with the first version of the plasmid, including the human FXN nucleotide sequence without a 5′ UTR FXN. No signal was visible on the anti-V5 blot, since no V5 epitope is included in the first plasmid version. Additionally, a signal was visible on the anti-frataxin blot. The anti-frataxin signal in lane 2 was less intense than the signal in lane 3.

Lane 4 shows results for SK-N-SH cells transfected with the fourth version of the plasmid, including the human FXN nucleotide sequence with a 5′ UTR FXN and with a V5 epitope tag. Signal was visible on the anti-V5 blot, and a signal was visible on the anti-frataxin blot. [000160] Lane 5 shows results for SK-N-SH cells transfected with the third version of the plasmid, including the human FXN nucleotide sequence without a 5′ UTR FXN and with a V5 epitope tag. Signal was visible on the anti-V5 blot, and a signal was visible on the anti-frataxin blot. The anti-frataxin signal and anti-V5 signal in lane 4 was less intense than the corresponding signals in lane 5.

These data provide evidence that expression of a human FXN nucleotide sequence can be modulated by inclusion of a 5′ UTR FXN sequence. Surprisingly, the 5′ UTR FXN sequence was found to decrease expression of the frataxin protein encoded by the plasmid construct, relative to a construct without the 5′ UTR sequence. Additionally, the effects in neuron-derived cells were consistent with the effects observed in Example 1.

### Example 3—Effects of the 5′ UTR FXN Sequence on FXN Gene Expression

An experiment was conducted to further investigate how expression of a human FXN nucleotide sequence in muscle-derived cells can be affected by inclusion of a 5′ UTR FXN sequence.

As in Examples 1 and 2, separate cell populations were transfected with each of the four plasmid versions. In the instant Example, C2C12 cells (a murine myoblast cell line) were used, instead of the HEK 293 cells of Example 1. Transfections and immunoblotting were performed as described in Example 1.

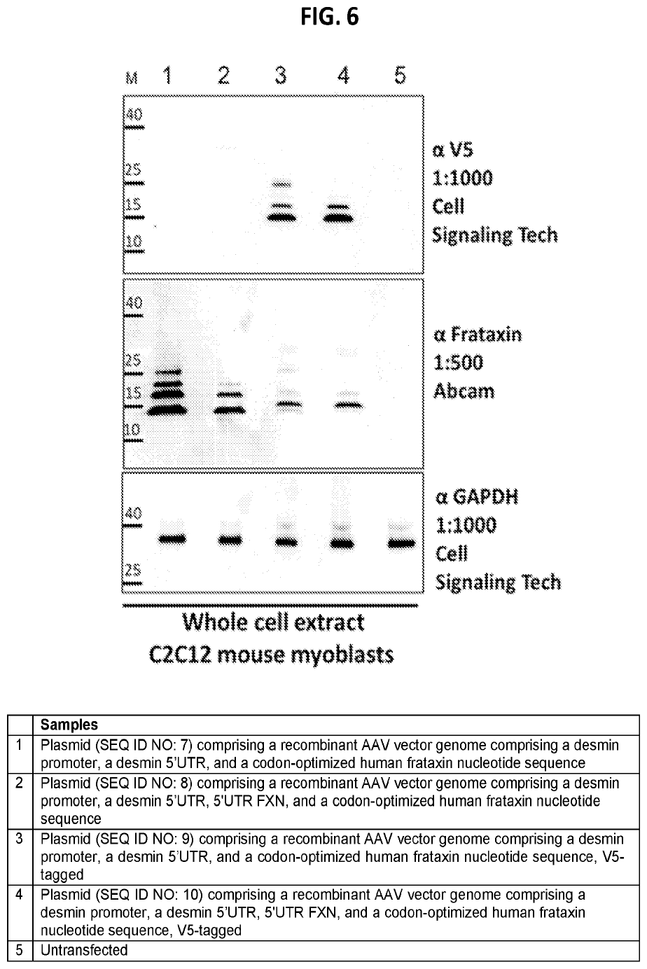

Referring to FIG. 6, lane 1 shows results for C2C12 cells transfected with the first version of the plasmid, including the human FXN nucleotide sequence without a 5′ UTR FXN. No signal was visible on the anti-V5 blot, since no V5 epitope is included in the first plasmid version. Additionally, a signal was visible on the anti-frataxin blot.

Lane 2 shows results for C2C12 cells transfected with the second version of the plasmid, including the human FXN nucleotide sequence with a 5′ UTR FXN. No signal was visible on the anti-V5 blot, since no V5 epitope is included in the second plasmid version. Additionally, a signal was visible on the anti-frataxin blot. The anti-frataxin signal in lane 2 was less intense than the signal in lane 1.

Lane 3 shows results for C2C12 cells transfected with the third version of the plasmoid, including the human FXN nucleotide sequence without a 5′ UTR FXN and with a V5 epitope tag. Signal was visible on the anti-V5 blot, and a signal was visible on the anti-frataxin blot.

Lane 4 shows results for C2C12 cells transfected with the fourth version of the plasmid, including the human FXN nucleotide sequence with a 5′ UTR FXN and with a V5 epitope tag. Signal was visible on the anti-V5 blot, and a signal was visible on the anti-frataxin blot. The anti-frataxin signal and anti-V5 signal in lane 4 was less intense than the corresponding signals in lane 3.

Lane 5 shows results for untransfected C2C12 cells. Signal from endogenous GAPDH is visible on the anti-GAPDH blot.

These data provide evidence that expression of a human FXN nucleotide sequence can be modulated by, inclusion of a 5′ UTR EX-N sequence. Surprisingly, the 5′ UTR FXN sequence was found to decrease expression of the frataxin protein encoded by the plasmid construct, relative to a construct without the 5′ UTR sequence. Additionally, the effects in muscle-derived cells were consistent with the effects observed in Examples 1 and 2.

### Example 4—Effects of the 5′ UTR FXN Sequence on Mitochondrial Function

An experiment was conducted to further investigate how expression of a human FXN nucleotide sequence in muscle-derived cells can affect mitochondrial function in cells expressing various FXN constructs.

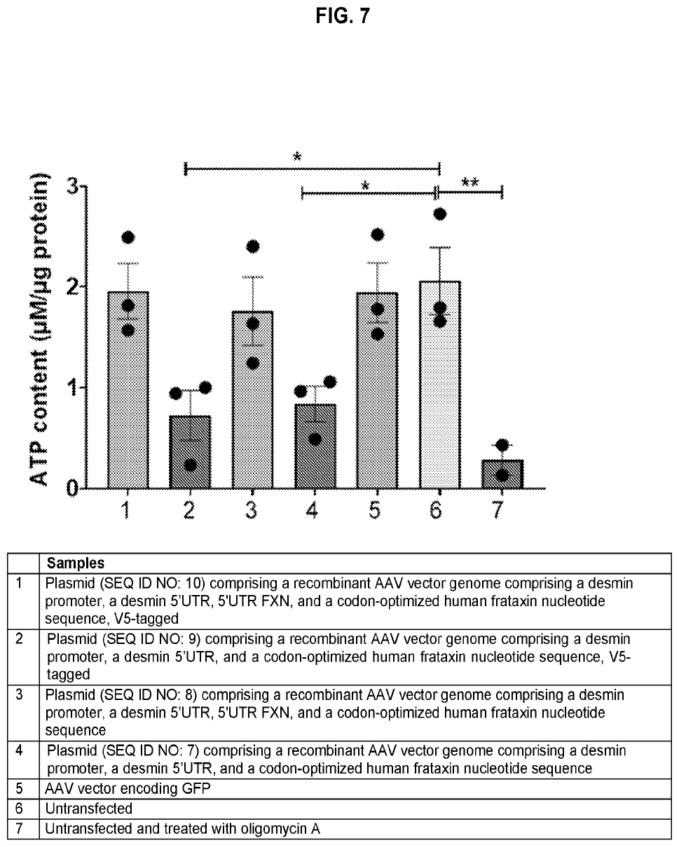

As in Examples 1-3, separate cell populations were transfected with each of the four plasmid versions. In the instant Example, C2C12 cells were used, instead of the HEK 293 cells of Example 1. Transfections for the four plasmid versions were performed as described in Example 1. 48 hours post-transfection, the four cell populations were collected and subjected to an adenosine triphosphate (ATP) assay. The ATP assay can measure ATP content in cells and can indicate the relative health of cells' mitochondria. After mitochondrial isolation, mitochondria were assayed using a luciferase assay to quantify the amount of ATP in each sample. Luciferase signal for each sample was analyzed with a standard curve of ATP concentration and measured relative to total protein in each sample. Referring to FIG. 7, data for cells transfected with these plasmids is shown as samples 1-4. Samples 5-7 are control data for cells transfected with a vector encoding green fluorescent protein (GFP), untransfected cells, and untransfected cells treated with oligomycin A, respectively. Oligomycin A is an inhibitor of ATP synthase, and cells treated with oligomycin A serve as a negative control for ATP production.

Sample 1 shows results for C2C12 cells transfected with the fourth version of the plasmid, including the human FXN nucleotide sequence with a 5′ UTR FXN and with a V5 epitope tag. The ATP concentration in sample 1 was not statistically different (one-way ANOVNA) from that of the untransfected cells of sample 6.

Sample 2 shows results for C2C12 cells transfected with the third version of the plasmid, including the human FXN nucleotide sequence without a 5′ UTR FXN and with a V5 epitope tag. The ATP concentration in sample 1 was increased relative to the ATP concentration in sample 2.

The ATP concentration in sample 2 was decreased relative to that of the untransfected control cells of sample 6. *P<0.05 (one-way ANOVA).

Sample 3 shows results for C2C12 cells transfected with the second version of the plasmid, including the human FXN nucleotide sequence with a 5′ UTR FXN. The ATP concentration in sample 3 was not statistically different (one-way ANOVA) from that of the untransfected cells of sample 6.

Sample 4 shows results for C2C12 cells transfected with the first version of the plasmid, including the human FXN nucleotide sequence without a 5′ UTR FXN. The ATP concentration in sample 3 was increased relative to the ATP concentration in sample 4. The ATP concentration in sample 4 was decreased relative to that of untransfected control cells of sample 6. *P<0.05 (one-way ANOVA).

The ATP concentration of sample 6 (untransfected cells), was significantly increased over the ATP concentration of sample 7 (oligomycin A-treated cells), confirming the ability of the assay to measure ATP availability. **P<,0.0 (one-way ANOVA).

These data provide evidence that expression of a human FXN nucleotide sequence which is unmodulated by the 5′ ITR FXN sequence can have deleterious effects for mitochondrial function. Surprisingly, inclusion of the 5′ UTR FXN sequence was found to reduce or eliminate the deleterious effects in muscle-derived cells.

### Example 5—Effects of the 5′ UTR FXN Sequence on FXN Gene Expression

An experiment was conducted to further investigate how expression of a human FXN nucleotide sequence in the C2C12 cells of Examples 3 and 4 can be affected by inclusion of a 5′ UTR FXN sequence in the context of various promoters.

Five plasmids encoding a recombinant AAV vector genome were constructed. A first plasmid (SEQ ID NO: 11) includes a codon-optimized human FXN nucleotide sequence (SEQ ID NO: 1) operably linked to a chicken beta actin (CBA) promoter sequence (SEQ ID NO: 5) and further includes a CBA 5′UTR (SEQ ID NO: 23) and 5′ UTR FXN sequence (SEQ ID NO: 2) operably positioned between the CBA promoter sequence and the human FXN nucleotide sequence. A second plasmid (SEQ ID NO: 1²) includes the codon-optimized human FXN nucleotide sequence operably linked to the CBA promoter sequence (SEQ ID NO: 5) and further includes a CBA 5′UTR (SEQ ID NO: 23) operably positioned between the CBA promoter sequence and the human FXN nucleotide sequence. A third plasmid (SEQ ID NO: 13) includes the codon-optimized human FXN nucleotide sequence operably linked to the desmin promoter sequence (SEQ ID NO: 4). The third plasmid further includes the 5′ UTR FXN sequence operably positioned between the desmin promoter sequence and the human FXN nucleotide sequence. A fourth plasmid (SEQ ID NO: 8) includes the codon-optimized human FXN nucleotide sequence operably linked to the desmin promoter sequence (SEQ ID NO: 4) and further includes a desmin 5 UTR (SEQ ID NO: 22) and 5′UTR FXN sequence (SEQ ID NO: 2) operably positioned between the desmin promoter sequence and the human FXN nucleotide sequence. A fifth plasmid (SEQ ID NO: 7) includes a codon-optimized human FXN nucleotide sequence (SEQ ID NO: 1) operably linked to a desmin promoter sequence (SEQ ID NO: 4) and further includes a desmin 5′UTR (SEQ ID NO: 22) operably positioned between the desmin promoter sequence and the human FXN nucleotide sequence.

After confining the accurate construction of the five plasmids via Sanger sequencing, each of the five versions were transfected into separate C2C12 cell populations, using commercially available transfection reagents according to the manufacturer's instructions.

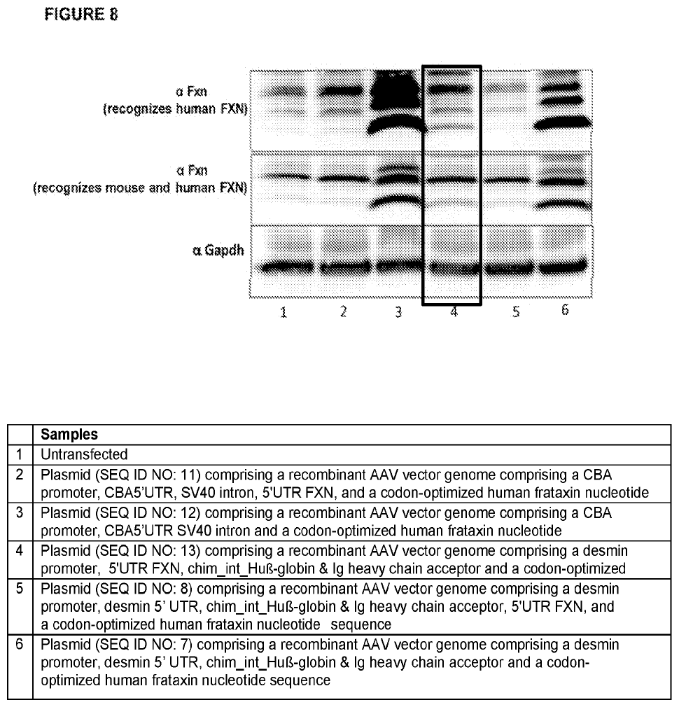

48 hours post-transfection the five cell populations were collected and total protein was harvested in RIPA buffer. Samples of the total protein (whole cell extract) were subjected to SDS-PAGE and immunoblotting. As indicated in FIG. 8, blots were probed using commercially available primary antibodies against either human frataxin or human and mouse frataxin (a. FXN), and, as a loading control, GAPDH (glyceraldehyde 3-phosphate dehydrogenase) (a GAPDH). Subsequently, blots were probed with HRP-conjugated secondary antibodies.

Referring to FIG. 8, lane 1 shows results for untransfected C2C12 cells. Signal from endogenous frataxin and GAPDH is visible on the two anti-frataxin blots and the anti-GAPDH blot, respectively.

Lane 2 shows results for C2C12 cells transfected with the first plasmid (SEQ ID NO: 11), including the CBA promoter—CBA 5′UTR—5′ UTR FXN—human FXN nucleotide sequence construct. Relative to lane 1, signal in lane 2 was increased.

Lane 3 shows results for C2C12 cells transfected with the second plasmid (SEQ ID NO: 12), including the CBA promoter—CBA 5′UTR—human FXN nucleotide sequence construct.

Relative to lane 2, signal in lane 3 was greatly increased.

Lane 4 shows results for C2C12 cells transfected with the third plasmid (SEQ ID NO: 13), including the desmin promoter—5′ UTR FXN—human FXN nucleotide sequence construct. Relative to lane 1, signal in lane 4 was increased.

Lane 5 shows results for C2C12 cells transfected with the fourth plasmid (SEQ ID NO: 8), including the desmin promoter—desmin 5′ UTR—5′ UTR FXN—human FXN nucleotide sequence construct. Relative to lane 4, signal in lane 5 was decreased. Relative to lane 1, signal in lane 5 was of a similar level.

Lane 6 shows results for C2C12 cells transfected with the fifth plasmid (SEQ ID NO: 7), including the desmin promoter—desmin 5′ UTR—human FXN nucleotide sequence construct. Signal in lane 6 was increased compared to any one of lanes 1, 4, or 5.

These data provide evidence that expression of a human FXN nucleotide sequence can be modulated by inclusion of a 5′ UTR FXN sequence. Surprisingly, the 5′ UTR FXN sequence was found to decrease expression of the frataxin protein encoded by the plasmid construct, relative to a construct without the 5′ UTR sequence. Additionally, evidence is provided that the effect of the 5‘ UTR FXN’ sequence on FXN expression is consistent for various promoters.

### Example 6—Effects of the 5′ UTR FXN Sequence on FXN Gene Expression

An experiment was conducted to further investigate how expression of a human FXN nucleotide sequence can be affected by inclusion of a 5′ UTR FXN sequence in the context of various promoters. In this experiment expression was investigated at the level of transcription using quantitative PCR (qPCR).

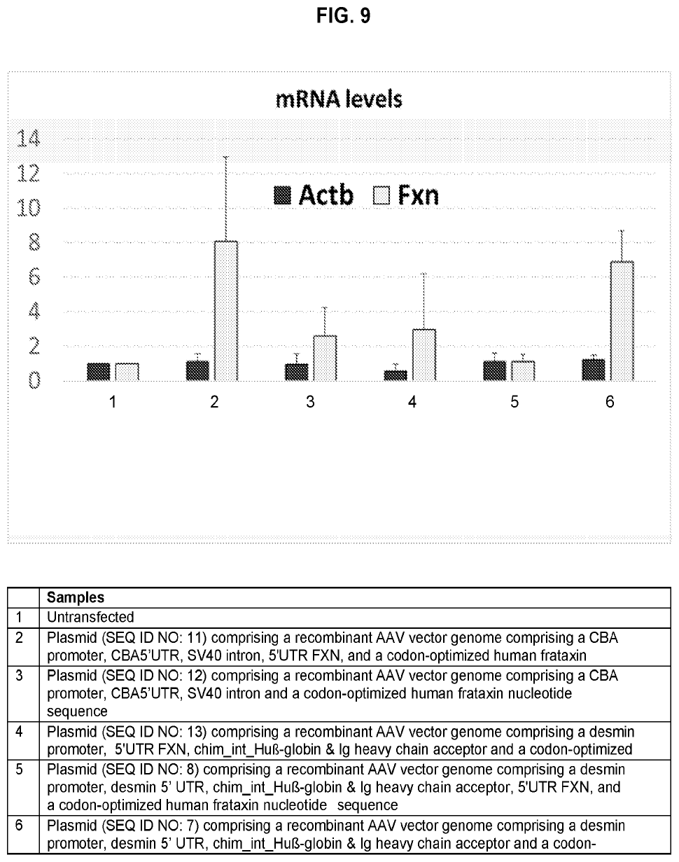

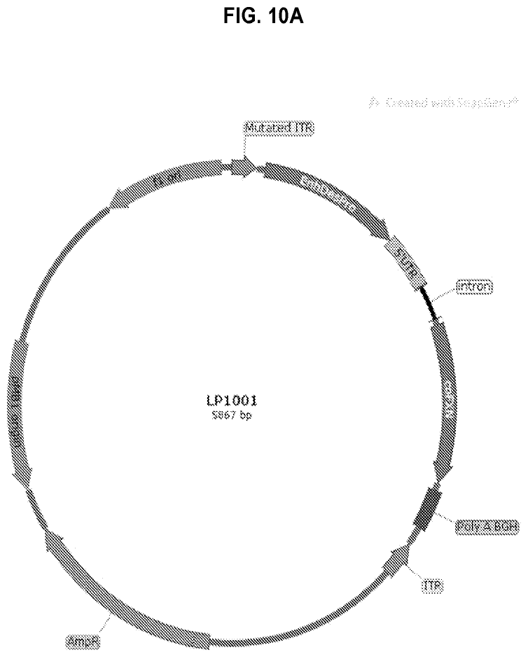

Cells transfected and harvested in Example 5, were also used to extract RNA samples. The RNA samples were then subjected to reverse transcription, and the resulting cDNA samples were then subjected to qPCR. The same five plasmids, as described in Example 5, were used in the present example. Referring to FIG. 9, relative expression of both beta-actin (Actb) and FXN (Fxn) were determined.

Sample 1, which was based on RNA extracted from untransfected cells, showed basal levels of both Actb and FXN expression.

Sample 2 was based on RNA extracted from cells transfected with the second plasmid (SEQ ID NO: 12), including the CBA promoter—CBA 5′UTR—human FXN nucleotide sequence construct. Sample 2 showed basal levels of Actb expression and increased levels of FXN expression, relative to the untransfected cells.

Sample 3 was based on RNA extracted from cells transfected with the first plasmid (SEQ ID NO: 11), including the CBA promoter—CBA 5′UTR—5′ UTR FXN—human FXN nucleotide sequence construct. Sample 3 showed basal levels of Actb expression and increased levels of FXN expression, relative to the untransfected cells, but decreased levels of FXN expression relative to Sample 2.

Sample 4 was based on RNA extracted from cells transfected with the third plasmid (SEQ ID NO: 13), including the desmin promoter—5′ UTR FXN—human FXN nucleotide sequence construct. Sample 4 showed basal levels of Actb expression and increased levels of FXN expression, relative to the untransfected cells.

Sample 5 was based on RNA extracted from cells transfected with the fourth plasmid (SEQ ID NO: 8), including the desmin promoter—desmin 5′ UTR—5′ UTR FXN—human FXN nucleotide sequence construct. Sample 5 showed basal levels of Actb expression and basal levels of FXN expression.

Sample 6 was based on RNA extracted from cells transfected with the fifth plasmid (SEQ ID NO: 7), including the desmin promoter—desmin 5′ UTR—human FXN nucleotide sequence construct. Sample 6 showed basal levels of Actb expression and increased levels of FXN expression, relative to the untransfected cells, Sample 4, and Sample 5.

These data provide evidence that expression of a human FXN nucleotide sequence can be modulated by inclusion of a 5′ UTR FXN sequence. Surprisingly, the 5′ LEER FXN sequence was found to decrease expression of the frataxin protein encoded by the plasmid construct, relative to a construct without the 5′ UTR sequence. Additionally, evidence is provided that the effect of the 5′ UTR FXN sequence on FXN expression is consistent for various promoters.

Sample 6 was based on RNA extracted from cells transfected with the fifth plasmid (SEQ ID NO: 7), including the desmin promoter—desmin 5′ UTR—human FXN nucleotide sequence construct. Sample 6 showed basal levels of Actb expression and increased levels of FXN expression, relative to the untransfected cells, Sample 4, and Sample 5.

These data provide evidence that expression of a human FXN nucleotide sequence can be modulated by inclusion of a 5′ UTR FXN sequence. Surprisingly, the 5′ UTR FXN sequence was found to decrease expression of the frataxin protein encoded by the plasmid construct, relative to a construct without the 5′ UTR sequence. Additionally, evidence is provided that the effect of the 5‘ U’TR FXN sequence on FXN expression is consistent for various promoters.

### Example 7

Consistent with the results obtained in Examples 1-6, cassettes containing 1) a tissue-restricted or ubiquitous promoter, and 2) FXN with or without the 5′ untranslated region (UTR) of FXN were designed and tested. The 5′UTR region was selected as a regulatory expression element based on the inherent structures of this region and effects on translation initiation (i.e. post-transcriptional control of gene expression). In vitro experiments demonstrated the effect of transfection-mediated overexpression of frataxin with or without the 5′UTR region of frataxin driven by a modified Desmin (DES) or Chicken Beta Actin (CBA) promoter in a self-complementary terminal repeat (TR) plasmid. Evaluation of AAV-Des driven 5′UTR-FXN (AAV-Des5′) overexpression in vivo was performed to determine if AAV-mediated overexpression of 5′UTR-FXN results in toxicity in wild-type (i.e. normal) mice following intravenous injection or dual-injection routes targeting cerebrospinal fluid (CSF) and skeletal muscle, administered via cisterna magna and intramuscularly (tibialis anterior muscle; TA), respectively.

In developing a gene therapy for FA, it is important to determine whether overexpression of FXN leads to toxicity and to determine whether expression may be regulated to limit toxicity and enhance overall therapeutic benefit. Previous studies reported FXN toxicity used the coding region of the gene without untranslated regions (UTR) that serve as regulatory expression elements to effect translation initiation (i.e. post-transcriptional control of gene expression). Therefore, whether gene expression could be controlled by including the 5′ untranslated region (5′UTR) of FXN was tested.

Additionally, for evaluation of translational efficiency in vitro using human fibroblast cell lines, two, different promoters, a modified human desmin (DES) promoter (Pacak et al. “Tissue specific promoters improve specificity of AAV9 mediated transgene expression following intra-vascular gene delivery in neonatal mice,” Genet Vaccines Ther 6, 13 (2008)) and a chicken β-actin (CBA) promoter (Shevtsova et al. “Promoters- and serotypes: targeting of adeno-associated virus vectors for gene transfer in the rat central nervous system in vitro and in vivo. Experimental Physiology, 90: 53-59 (2005)), were tested, to drive expression of FXN. DES is known for its high transduction in the myocardium, skeletal muscle and CNS, while CBA is a strong ubiquitous promoter leading to high transduction in all cell types. By comparing two different promoters, the aim was to optimize translational efficiency of FXN levels without inducing FXN toxicity. To evaluate FXN overexpression in viva, a AAV8 triple-capsid mutant, AAV87TM (SEQ ID NO:61), was used for delivery of DES-driven 5′UTR-FXN In wild-type mice (AAV8TM-DES-5′UTR-FXN) (plasmid LP1001) (Gilkes et al. “Mucopolysaccharidosis IIIB confers enhanced neonatal intracranial transduction by AAV8 but not by 5, 9 or rh10. Gene Ther. 2016; 23(3):263-27I). This was performed in parallel with in vitro studies to evaluate potential toxicity following FI overexpression.

### Agents

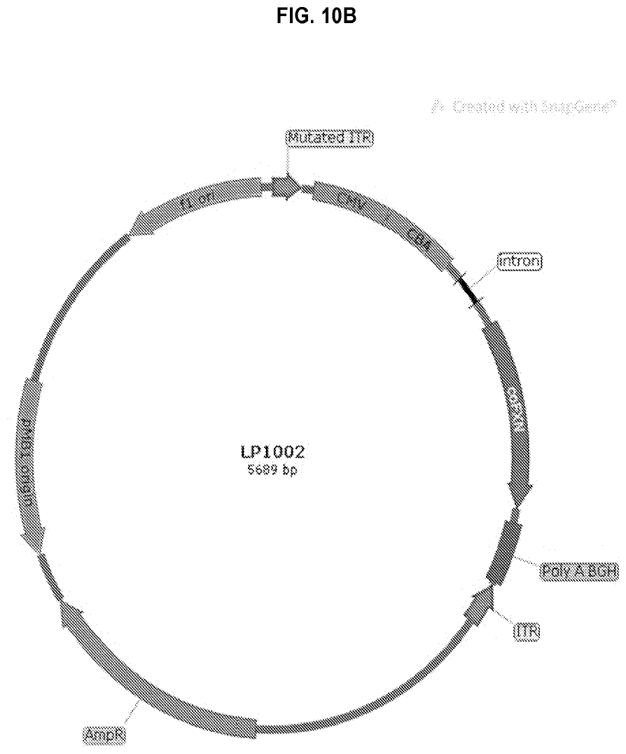

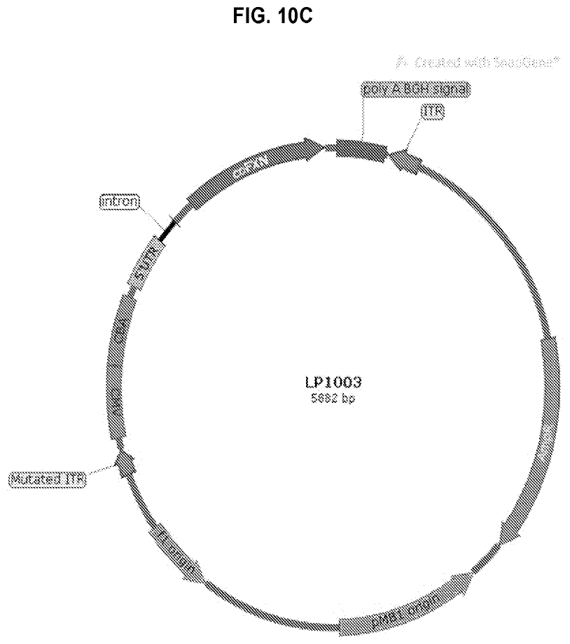

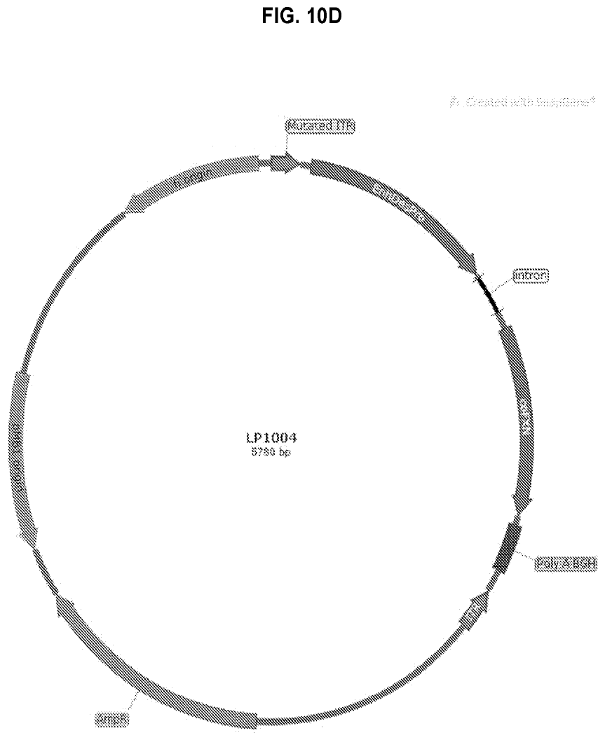

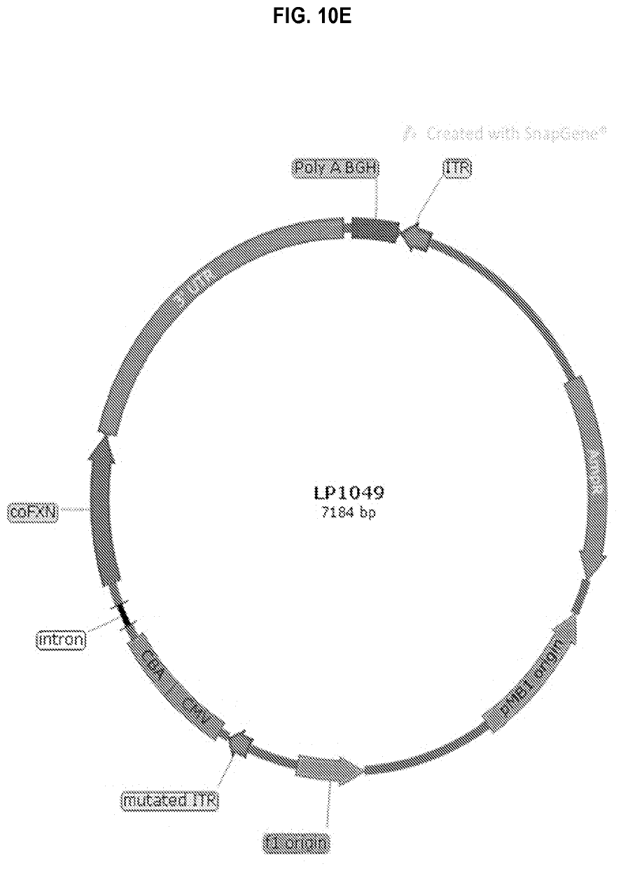

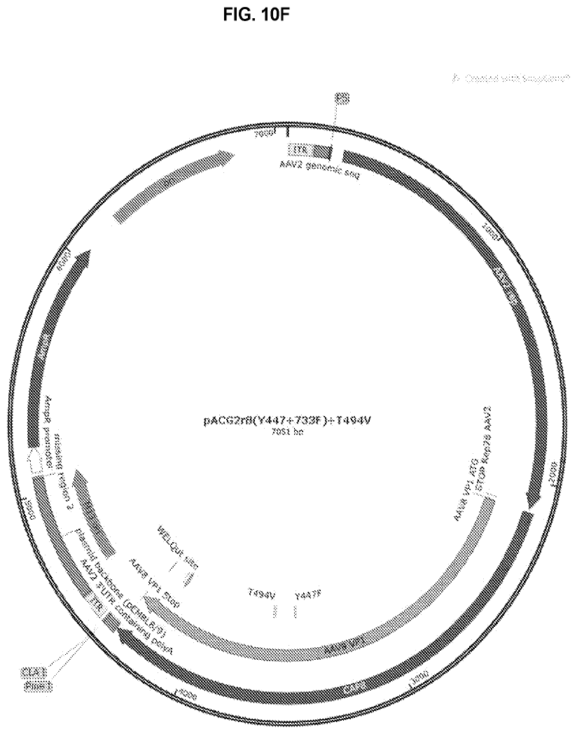

Human codon-optimized frataxin (630 bp) 3′UTR or 5′UTR human frataxin (1490 bp and 221 bp, respectively) were cloned in a self-complementary pTR plasmid. The genes of interest (GOI) were driven by a modified human desmin (DES) promoter or chicken β-actin (CBA) promoter (see Table 2). Plasmid maps are shown in FIGS. 10A-E. The SEQ ID NO. for the nucleic acid sequence of each plasmid is also provided in Table 2.

TABLE 2

| Plasmid List | | |
|---|---|---|
| | Plasmid | |
| Plasmid ID | Description. | SEQ ID NO. |
| pLP1001 | DES-5′UTR-FXN | 24 |
| pLP1002 | CBA-FXN | 25 |
| pLP1003 | CBA-5′UTR-FXN | 26 |
| pLP1004 | DES-FXN | 27 |
| pLP1049 | CBA-3′UTR-FXN | 28 |

GOIs were synthesized by Integrated DNA Technologies (IDT; Coralville, Iowa, USA) and cloned into pTR-plasmid by restriction enzymes. pLP1001 was generated by cloning “EnhDesPro-intron-5′UTRcoFXNv1” fragment into pds AAV-CBA-EGFP (GenBank: Accession No. MK225672 (SEQ TE NO: 29)) using the restriction enzymes KpnI and SacI. pLP1004 was synthesized by cloning “EnhDesPro-intron-coFXNv1” into pds AAV-CBA-EGFP using restriction site KpnI and SacI. pLP1002 was completed by synthesizing coFXNv1 with Age1 and Sac1 restriction sites which was then cloned into pds-AAV-CBA-EGFP. pLP1003 was completed by synthesizing 5′UTR-intron and cloning it into LP1002 using SalI and SpeI. (Lacerta Therapeutics, Inc. Intellectual Property. Lab notebooks LBN24 LBN25, LBN08)

All plasmids were cloned, transformed, and verified by restriction enzyme digest Sma I at Lacerta Therapeutics, Inc. (Alachua, Fla., USA). Following sequence verification (Eurofins), plasmids were sent to Aldevron (Fargo, N. Dak., USA) for large-scale plasmid production. AAV8TM virus expressing DES-5′UTR-FXN (AAV8TM-DES-5′UTR-FXN) was produced in 2 cell stacks (Reference Number 7047 and 7048) by triple transfection in adherent HEK293 cells at the University of Florida, Powell Gene Therapy Center (PGTC), Vector Core Laboratory (Gainesville, Fla., USA). The two cell stacks were pooled, virus was purified by Iodixanol gradient centrifugation followed by an AAVX column and titered by dot blot at PGTC (Table 3). Vector titer was also determined by digital drop PCR (ddPCR) at Lacerta. However, dot blots reportedly show elevated titers and less accuracy when compared to digital drop PCR. Therefore, in vivo dosing was calculated by the ddPCR titer.

TABLE 3
AAV8TM-DES-5′UTR-FXN viral titer determined
by dot plot (PGTC) or ddPCR (Lacerta)

| | Titer | Titer | Final Volume | |
|---|---|---|---|---|
| Method | (vg/ml) | (vg/μl) | (mL) | Total vg |
| ddPCR | 1.57E+13 | 1.57E+10 | 0.86 mL | 1.35E+13 |
| Dot Blot | 2.54E+13 | 2.54E+10 | 1 mL | 2.54E+13 |

### Dose and Exposure

LTX 401.3: Intravenous Administration of AAV8TM-DES-5′UTR-FXN to Assess Potential Toxicity from FXN Overexpression in Normal Wild-Type Mice

Each animal (n=3) received a single bolus injection of 5E±13 vg/kg AAV8TM-DES-5′UTR-FXN in a final volume of 100 μL through the jugular vein. Vims was prepared by dilution in PBS+0.001% pluronic (excipient) to attain the final dose formulation. An untreated, age matched control animal was also used in this study.

LTX 401.4: Dual Administration of AAV8TM-DES-5′UTR-FXN Via Intra Cisterna Magna and Intramuscular Injection to Assess Potential Toxicity from FXN Overexpression in Normal Wild-Type Mice

For intra cisterna magna (ICM) injections, female mice received a single injection of 1E+11 vg (n=3), and males received 1.5E+11 vg total (n=3) in a final volume of 10 μl. Virus was diluted in excipient to attain the final dose formulation. In addition, these mice received intramuscular injections (IM) at three different doses (3.70E+8, 8.2E+8, or 1.92E+9 vg/mg tibialis anterior (TA)). Each dose was injected into the right and left TA of one male and one female mouse. To calculate dosage, TA muscle weight was assumed to be 10% of the total body weight. All IM injections were prepared to a final volume of 5 μl by quantity sufficient dilution in excipient. One animal was injected with 10 μL excipient as a procedural control. In addition, an untreated, age matched control animal was included for comparison.

### Research Objectives and Rationale

The overall goal for this set of experiments was to determine the potential toxicity of FXN overexpression. In addition, in vitro experiments with human fibroblast cell lines were performed to test the hypothesis that addition of a 5′ untranslated region upstream of FXN will regulate gene expression and reduce any potential toxicity as reported previously. In vivo experiments evaluated biodistribution and potential toxicity in normal wild-type mice using multiple routes of administration and dosages. Combined, this data provides support for the regulation of gene expression, potential toxicity, and capsid biodistribution.

### Study Design

### Human Fibroblast Toxicity Analysis

Two healthy controls and two patient fibroblast cell lines (Table 4) were transfected with the frataxin plasmids listed in Table 2. Following plasmid transfection, assays were performed for cellular toxicity (measured by DNA content), ATP quantification (mitochondrial status), and Western Immunoblot and ELISA for human FXN.

TABLE 4
Fibroblast cell lines obtained from Corielle Institute for in vitro assessments

| | | | | | | | Age at |
|---|---|---|---|---|---|---|---|
| ID # | Affected | Product | Source | Gene | Mutations | Gender | Sample |
| GM04078 | Friedreich | Fibroblast | Skin, Arm | FXN | (GAA)ₙ | Male | 30 yr. |
| | Ataxia 1 | | | | Expansion | | |
| GM03816 | Friedreich | Fibroblast | — | FXN | (GAA)ₙ | Female | 36 yr. |
| | Ataxia 1 | | | | Expansion | | |
| GM00969 | No | Fibroblast | Skin, | — | — | Female | 2 yr. |
| | | | Unspecified | | | | |
| GM03651 | No | Fibroblast | Skin, Arm | — | — | Female | 25 yr. |

LTX 401.3: Intravenous Administration of AAV8TM-DES-5′UTR-FIN to Assess Potential Toxicity from FXN Overexpression in Normal Wild-Type Mice

The experimental design for intravenous administration of AAV8TM-DES-5′UTR-FXN (AAV-DES5′) is outlined in Table 5. Wild-type C57BL/6J mice (JAX, 000664) were harvested 28-32 days post-injection and tissues were collected as described below, Table 8. Tissues were processed for FXN detection by histology or ELISA to determine biodistribution, human frataxin protein expression, or obvious toxicity following vector administration.

TABLE 5
Experimental Design for 401.3, Intravenous
administration of AAV8TM-DES-5′UTR-FXN to assess
potential toxicity from FXN overexpression in normal wild-
type mice

| | | | Route | | |
|---|---|---|---|---|---|
| Mouse | | | of | | |
| Strain | Treatment | Dose | Admin. | Gender | n |
| B6/J | Uninjected | — | — | Male | 1 |
| B6/J | AAV- | 5E+13 | IV | Male | 3 |
| | DES5′ | vg/kg | | | |

LTX 401.4: Dual Administration of AAV8TM-DES-5′UTR-FXN Via Intra Cisterna Magna and Intramuscular Injection to Assess Potential Toxicity from FXN Overexpression in Normal Wild-Type Mice

Three animals of each gender were injected with a single ICM dose plus one of three IM doses of AAV8TM-DES-5′UTR-FXN (AAV-DES5′) per the experimental design outlined in Table 6. For control tissues, one animal of each gender vas injected with excipient only and one animal of each gender was untreated (WT). Animals were harvested 28-32 days post-AAV administration. Tissues were harvested according to section 4.5.1, Table 9. Tissues were analyzed for human FXN by histology and ELISA.

TABLE 6
Experimental Design for 401.4, Intra cisterna magna
plus Intramuscular administration of AAV8TM-DES-5′UTR-
FXN to assess potential toxicity from FXN overexpression in
normal wild-type mice

| | | | Route | | |
|---|---|---|---|---|---|
| Mouse | | | of | | |
| Strain | Treatment | Dose | Admin. | Gender | N |
| B6/J | Untreated | N/A | N/A | Male | 1 |
| B6/J | Excipient | 10 μL | ICM | Male | 1 |
| | | 5 μL | IM | | |
| B6/J | AAV- | 3e11 vg/g brain | ICM | Male | 1 |
| | DES5′ | 3.7e+08 vg/mg TA | IM | | |
| B6/J | AAV- | 3e11 vg/g brain | ICM | Male | 1 |
| | DFS5′ | 8.2e+08 vg/mg TA | IM | | |
| B6/J | AAV- | 3e11 vg/g brain | ICM | Male | 1 |
| | DES5′ | 1.92e+09 vg/mg TA | IM | | |
| B6/J | Untreated | N/A | N/A | Female | 1 |
| B6/J | Excipient | 10 μL | ICM | Female | 1 |
| | | 5 μL | IM | | |
| B6/J | AAV- | 2e11 vg/g brain | ICM | Female | 1 |
| | DES5′ | 3.7e+08 vg/mg TA | IM | | |
| B6/J | AAV- | 2e11 vg/g brain | ICM | Female | 1 |
| | DES5′ | 8.2e+08 vg/mg TA | IM | | |
| B6/J | AAV- | 2e11 vg/g brain | ICM | Female | 1 |
| | DES5′ | 1.92e+09 vg/mg TA | IM | | |

Analysis of Frataxin Overexpression in Human Fibroblast Cell Lines to Understand their Effect on Toxicity and Potential Disruption in Mitochondrial Function

### Cell Couture of Human Fibroblasts

Human fibroblast cell lines from Friedreich's Ataxia patients (ID #GM04078 and GM-03816) and healthy donors (ID #GM00969 and GM03651) were obtained from the Corielle Institute (Camden, N.J., USA: Table 7) and cultured in fibroblast growth medium (Promocell C-23010) with 20% fetal bovine serum (Atlanta Biologicals, 511150H). 50 units/ml penicillin, and 50 mg/ml streptomycin (Gibco. 15140-122).

TABLE 7
Fibroblast cell lines obtained from Corielle Institute for in vitro assessments

| | | | | | | | Age at |
|---|---|---|---|---|---|---|---|
| ID # | Affected | Product | Source | Gene | Mutations | Gender | Sample |
| GM04078 | Friedreich | Fibroblast | Skin, Arm | FXN | (GAA)ₙ | Male | 30 yr. |
| | Ataxia 1 | | | | Expulsion | | |
| GM03816 | Friedreich | Fibroblast | — | FXN | (GAA)ₙ | Female | 36 yr. |
| | Ataxia 1 | | | | Expansion | | |
| GM00969 | No | Fibroblast | Skin, | — | — | Female | 2 yr. |
| | | | Unspecified | | | | |
| GM03651 | No | Fibroblast | Skin, Arm | — | — | Female | 25 yr. |

### Transfection of Human Fibroblasts

Approximately 24 hours before transfection, cells were seeded in a 6 well-plate at a density of 0.8-3.0×105 cells/nil in 2.5 ml complete growth medium per well. Cells were maintained at 5% CO2, 37° C. overnight, and the next day were transfected with 1.25, 2.5 or 5 μg of plasmid for titration experiments and 5 μg of plasmid in all other in vitro experiments (listed in Table 2) using TransIT@-LT1 Transfection Reagent (Mirus Bio, MIR 2300) according to the manufacturer's protocol.

### Measurement of Cellular Toxicity in Human Fibroblasts by Proliferation Assay

Cells were harvested 24 hours after transfection with a cell lifter, then counted and seeded into a 96-well plate at cell density of 5.000 cells/well. The next day, a CyQUANT™ Cell Proliferation Assay (Invitrogen, C7026) was performed according to manufacturer's protocol.

### Measurement of ATP Content in the Mitochondrial Fraction of Human Fibroblasts

Cells were harvested and processed for mitochondrial isolation as mentioned in Preble et al. (“Rapid isolation and purification of mitochondria for transplantation by tissue dissociation and differential filtration,” J Vis Exp. 2014; (91):e51682). Protein concentration of the mitochondrial fraction was measured by DC assay (Bio-Rad, 5000112). ATP content was measured with ATPlite Luminescence Assay System (PerkinElmer. 6016943). In this assay, luminescence is proportional to the ATP concentration in the sample. Briefly, isolated mitochondria (10 μl) were seeded into 96-well plates, then lysed with mammalian cell lysis solution (50 μl) lyse mitochondria and release ATP. Luminescence was measured using a CLARIOstar Microplate Reader (BMG Labtech). A standard curve was generated per the manufacture's protocol and the ATP concentration for each sample was obtained by linear regression analysis. ATP content was normalized to mitochondrial protein concentration. See, Saha et al. “Impact of PYROXD1 deficiency on cellular respiration and correlations with genetic analyses of limb-girdle muscular dystrophy in Saudi Arabia and Sudan,” Physiol Genomics. 2018; 50(11):929-939.

### Quantification of Human FXN in Mitochondrial Fraction of Human Fibroblasts

Protein concentration was determined by Detergent Compatible (DC) Protein Assay (Bio-Rad). For Western immunoblot, mitochondrial extract in the amount of 200 μg total protein was resolved on a 4-12% tricine-polyacrylamide gel (Life Technologies), then transferred onto a nitrocellulose membrane (20 μm). The membrane was blocked in 5% milk/TBST (0.5% Tween-20, 8 mM Tris-Base, 25 mM Tris-HCl, 154 mM NaCl), then probed with primary mouse anti-frataxin antibody (supernatant) at a 1:1,000 dilution and anti-GAPDH at a 1:1,000 dilution (2118S. Cell Signaling Technologies). The membrane was incubated with horseradish peroxidase-conjugated secondary antibodies and visualized by chemiluminescence (Millipore) on an iBright CL1000. To determine human frataxin levels in cultured fibroblasts, mitochondrial extracts were assayed using Human Frataxin ELISA Kit (ab176112), according to the manufacturer's instructions.

### Densitometric Analysis

Quantification of Western blot images was conducted using ImageJ (Gassmann et al. “Quantifying Western blots: Pitfalls of densitometry, ELECTROPHORESIS, 30: 1845-1855. doi:10.1002/elps.200800720). FXN levels were normalized to GAPDH levels.

### Indirect Immuno-Staining of Patient Fibroblasts

Fibroblasts were seeded onto chamber slides (Thermo Scientific, 12-565-8) treated with 10% Matrigel (Corning, CB-40234A) in Dulbecco's Modified Eagle's Medium (Corning #3MT10013CV) after transfection with plasmids, as indicated. At day 4, the growth medium was removed. Cells were washed in PBS, then fixed with 2% Paraformaldehyde in PBS for 10 minutes at room temperature and consecutively washed in PBS for three times. Cells were permeabilized with 0.2% Triton X-100 in PBS for 10 minutes and blocked by the addition of 5% normal goat serum in PBS for 60 minutes. After overnight incubation at 4° C. with mouse anti-human FXN (Puccio) in a 1:100 dilution and rabbit anti-Tomm20 (Cell Signaling Tech, 42406S) in a 1:150 dilution, cells were extensively washed and further incubated with goat anti-mouse 488 and goat anti-rabbit 594 (1:1000 each) secondary antibody. Cells were washed three times with PBS and coverslips were mounted on a microscope slide using a VECTASHIELD® Vibrance mountant with DAPI (Vector Laboratories, H-1200-10). Microscopic analysis was performed using a Keyence BZ-X810 fluorescence microscope. Immunostaining was also performed with untreated control fibroblasts to determine the specificity of the frataxin signal and to optimize antibody mediated FXN detection.

### In Vivo Assessment of FXN Overexpression in Wild-Type Mice

### Virus Titering

The titer (vg/mL) for AAV8tm-DES-5′UTR-FXN was determined by dot blot at PGTC and by the QX200 Droplet Digital PCR System from Bio-Rad (QX 200 Droplet Generator and QX200 Droplet Reader Bio-Rad). For ddPCR, samples were serially diluted in Nuclease Free Water to 1E3 to 1E2 vg/well to ensure the samples were below the maximum range of analysis (1E4 vg/well). To ensure enough volume for droplet formation, a total volume of 25 uL Mastermix and sample was prepared. The reaction mixture included 1× ddPCR Supermix for Probes (No dUTP; Bio-Rad 1863024), 900 nM BGH forward 5′ GCC AGC CAT CTG TTG T 3′ (IDT) (SEQ ID NO: 30) and reverse 5′ GGA GTG GCA CCT TCC A 3′ (IDT) (SEQ ID NO: 31) primers, 250 nM BGH probe 5′ FAM/TCC CCC; GTG/ZEN/CCT TCC TTG ACC/ABkFQ 3′ (IDT) (SEQ ID NO: 32), and 5 μL of sample diluted in nuclease free water. The mixture was vortexed prior to droplet preparation. Droplets were formed using the QX200 Droplet Generator (Bio-Rad) by adding 20 μL of the sample mixture into the center wells of a DG8 Cartridge (Bio-Rad, 1864008) followed by 70 μL of Droplet Generation Oil for Probes (Bio-Rad, 1863005) into the appropriate wells of the cartridge. The cartridge was covered with a DG8 Gasket (Bio-Rad, 1863009) and placed into the Droplet Generator. Newly formed droplets (40 μL) were carefully pipetted and transferred to a ddPCR 96-well plate (Bio-Rad, 12001925) and covered with a Pierceable Foil Heat Seal (Bio-Rad, 1814040), placed in a PX1 PCR Plate Sealer (Bio-Rad) and heat sealed at 180° C. for 5 seconds. The plate was immediately removed and placed in a C1000 Thermal Cycler (Bio-Rad) at 95° C. for 10 minutes, then 95° C. for 30 seconds, 57.4° C. for 1 minute, and 72° C. for 15 seconds for 42 cycles, followed by 98° C. for 10 minutes and an indefinite hold of 12° C. until the un was stopped. After completed PCR, the plate was transferred to the QX200 Droplet Reader (Bio-Rad) for Absolute Quantification analysis. Results were reported as a concentration and copies per 20 μL well. To determine the number of vector genomes per mL, the formula {((Concentration*total volume initial reaction)/μL Sample)*1000*dilution factor)}] was used.

### Surgical Suite Set Up

Before surgical procedures, the procedure space was prepared with three designated stations: the animal preparation area, the surgical area, and the recovery/post-op area (Gakuba et al., “General Anesthesia Inhibits the Activity of the “Glymphatic System”,” Theranostics, 8(3), 710-722 (2018)). The Animal Prep Area: To reduce the chance of microbial contamination of the sterile surgical field, the animal prep area was positioned on a designated table away from the surgical area. Mice were anesthetized using 2% isoflurane (IL O2) (Falk et al. “Comparative impact of AAV and enzyme replacement therapy on respiratory and cardiac function in adult Pompe mice, Molecular Therapy—Methods & Clinical Development, Volume 2, 2015, 15007, ISSN 2329-0501, https://doi.org/10.1038/mtm.2015.7) was administered through a chamber using a vaporizer system, the animal was weighed, and its hair was clipped. The animal was then moved to the surgical area.

### Surgical Area

The surgical area equipment consists of a stainless-steel table, mobile vaporizer anesthesia system, glass bead sterilizer, stereotaxic device, and injection pump; all surfaces were cleaned with 70% alcohol prior to surgery. A sterile drape was placed underneath the stereotax. A heating pad with digital readout was placed on the stereotax where the animal was to be placed and a puppy pad is wrapped around the heating pad once to prevent direct contact of the animal to the heating pad. Two specimen cups, one with chlorhexidine surgical wash, and one with sterile saline rinse were placed on the sterile drape as well as the autoclaved surgical instruments. Surgical instruments were cleaned with soap and water, dried, and sterilized with the glass bead sterilizer in between animals. Autoclaved instruments were used and cleaned a maximum of 10 times before switching to a new set of autoclaved instruments.

### Post-Op Recovery Area

Upon completion of the surgical procedure, the animal was moved to a clean cage for monitoring and post-op care. This cage was set up so that half of the cage rests on a heating pad with digital readout to minimize hypothermia in the recovering animal; there was a puppy pad between the cage and heating pad to prevent direct contact. The recovery station was close enough to the surgeon/assistant so that recovery could be monitored. Once the animal was able to move normally on its own, showed no sign of distress or pain, and was otherwise bright, alert, and responsive; the animal was be moved back to the cage rack. Animals were monitored daily for the first 5 days post-op, then checked at least every other day until harvest to monitor for complications.

LTX-401.3: Intravenous Injection of AAV8TM-DES-5′UTR-FXN into Wild Type Mice

### Pre-Operative Mouse Preparation

At the preparation station, animals were anesthetized using vaporized Isoflurane as outlined in the approved IACUC protocol and then weighed (g) in order to calculate analgesia (Rimadyl) administration. Hair was carefully removed from the neck/throat area using depilatory cream (Nair™) and the animal was transferred to the surgical area.

### Surgical Set-Up

After pre-op, the animal was given 1 mL of Lactated Ringers (to replace fluid loss during surgery) and a 10 mg/kg dose of analgesia (Rimadyl) subcutaneously. To begin the surgical procedure, the animal was placed on the stereotaxic stage in the supine position with its face positioned upwards into the anesthesia face mask. The head was held in an upward position using the anesthetic face mask, the front feet were pulled gently downward and secured in place with tape to expose the neck of the animal and keep contaminated paws out of the surgical area. The flow of vaporized Isoflurane was transferred from the induction chamber at the pre-op station to the anesthesia mask. Anesthetic plane was assessed frequently throughout surgery by observing respirations as well as a toe pedal response, isoflurane levels were adjusted accordingly.

After appropriate positioning of the animal, the surgical site was aseptically prepared using alternating spiraling outward scrubs of chlorhexidine and 0.9% sterile saline solution beginning at the center of the area from which hair vas removed and working outward towards the periphery, this is repeated at least three tines, or until there is no debris seen on the swab. After the area was sterilized, a 2 cm incision in the skin was made using sterile surgical scissors and forceps to expose the jugular vein. The jugular vein vas located by gently moving away superficial connective and adipose tissue from the incision around the animal's neck. The animal was then ready for injection.

### Injection

Once the jugular vein was exposed to the surgeon, a primed and prefilled 29-gauge insulin syringe 100 μl of diluted virus was inserted to the vein with the bevel of the needle facing upwards. Before injecting any virus, the syringe was aspirated checking for blood flowback into the syringe. If there was no blood drawn up into the syringe, the needle was repositioned and checked again. If there was still no blood return, the needle was removed, and a fresh attempt was made. If excessive bleeding occurred, sterile cotton swabs were used to apply pressure to the vein until bleeding was stopped. In the unlikely event that the vein appeared to be unusable prior to injection, the site was sutured, and the surgeon performed the injection on the other side of the neck, this was noted on the surgery record. Once injection was complete, the syringe was slowly retracted, and pressure was applied to the injection site with a sterile cotton swab to prevent back flow and bleeding; the site vas then cleaned and sutured.

Isoflurane delivery was stopped, and the animal was removed from the stereotaxic device. Initial recovery was monitored on the surgical stage before moving the mouse into the recovery cage.

LTX-401.4: Intra Cisterna Magna and Intramuscular Injection of AAV8TM-DES-5′UTR-FXN into Wild Type Mice

### Pre-Operative Mouse Preparation

After pre-op, the animal was placed on the stereotaxic device by fixing the head in ear bars and placing the nose in the integrated anesthetic mask. The flow of vaporized Isoflurane was transferred from the induction chamber to the stereotaxic anesthesia mask. Anesthetic plane was assessed frequently throughout surgery by observing respirations as well as a toe pedal response; isoflurane levels were adjusted accordingly. To minimize the chance of respiratory distress, gauze was placed under the heating pad to lift the mouse at an angle so that the spine formed a downward 15° angle with the horizontal line of the ear bars. The anesthetic mask was then adjusted so that the facial surface formed a 15° angle with the vertical line of the stereotaxic arm, this achieves an approximated 90° angle of the head to the spine. At this position, the cisterna magna was the highest point of the animal's body and the dura was taut to allow puncture and prevent viral backflow. After appropriate positioning of the animal, the surgical site was aseptically prepared using alternating spiraling outward scrubs of chlorhexidine and 0.9% sterile saline solution beginning at the center of the shaved area and working outward towards the periphery, this was repeated at least three times or until there was no debris seen on the swab. After the area was sterilized, a 2 cm incision in the skin was made using sterile surgical scissors and forceps to expose the suboccipital muscles covering the cisterna magna. These muscles were gently separated using forceps (to ensure minimal to no muscle damage is caused) and held to the side with Dieffenbach Serrefine vascular clamps, thereby exposing the surface of the dura mater. The animal was then given 1 mL of Lactated Ringers (to replace fluids lost during surgery) and a 10 ng/kg dose of analgesia (Rimadyl) subcutaneously. The animal was then ready for injection.

### Surgical Set-Up

A 25 μl Hamilton syringe with a 33-gauge 45°-degree beveled needle attached, pre-filled with 12 μl (ensuring sufficient volume to deliver 10 μl) of diluted virus was then placed in the injection pump, mounted on the stereotaxic arm. Subsequently the stereotaxic arm was moved from a 90° vertical angle, down to a 45° angle towards the surgeon. This positioned the needle to be perpendicular to the dura mater. Then the needle of the syringe was positioned using the micromanipulator dials to touch the dura mater (avoiding any blood vessels), the digital readout of the stereotaxic device is then zeroed to mark the start of the dura. With a quick, small rotation of the dorsoventral dial, the dura mater was pierced. The needle was then retracted back out of the dura using the dials to allow the outflow of cerebrospinal fluid (CSF) to create negative pressure to allow room for the virus. The outflow of CSF also confirmed that the surgeon was in the correct location. Once the flow of CSF was confirmed by the surgeon, the needle was then reinserted using the dials to position the needle bevel just inside the cisterna magna, approximately 1 mm deep past the recorded dura location. Once the needle was in the correct position, the whole stereotaxic frame wars slowly elevated to form a 30° angle with the table surface to promote the downward flow of virus into the brain. A dollop of sterile Vaseline was placed entirely around the needle at the injection location and on the exposed dura mater to help prevent back flow of vims and CSF. The injection pump, set at 1000 nl/min, was then started, and precisely delivered 10 μl of diluted virus.

Once the viral load was delivered, a tinier was set for one minute to allow for the virus to flow through the subarachnoid space with the CSF to reduce the chance of virus backflow when removing the needle from the cisterna magna. The needle is carefully retracted using the dorsoventral dial. After the needle was retracted, the stereotaxic device was carefully repositioned back to the table level, and the surgical area was cleaned and sutured.

Following the ICM injection, anesthetized animals (still on the stereotax) underwent tibialis anterior muscle injections in the left and right leg. The injection site was aseptically prepared using alternating spiraling outward scrubs of chlorhexidine and 0.9% sterile saline solution beginning at the center of the Naired area and working outward towards the periphery, this was repeated at least three times or until there was no debris seen on the swab. Injections were performed into the central portion of the tibialis anterior muscle using a primed 0.5-ml tuberculin syringe with a 29-gauge 45°-degree beveled needle. The needle was inserted into the skin, bevel up, with the needle nearly parallel to the plane of the skin. Once the surgeon was confident in needle positioning into the muscle, the viral load was slowly injected. Once the contents of the syringe were fully injected, the needle was slowly retracted to reduce viral backflow. Pressure was applied to the injection site directly after the needle was retracted to help prevent back flow.

### Injection

At the preparation station, animals were anesthetized using vaporized Isoflurane as outlined in the approved IACUC protocol and then weighed (g) in order and calculate the amount of analgesia (Rimadyl) needed. Hair was then removed from the back of the head extending from just behind the eyes to the base of the neck using electric clippers. Hair on both the lower hind limbs was then carefully removed using Nair to expose the tibialis anterior muscles. The animal was then transferred to the surgical area.

### Tissue Harvest

Animals were euthanized using an overdose of vaporized isoflurane. Once respirations ceased, the animal was placed on the harvest table and pinned into place onto a Styrofoam board. The abdominal and thoracic cavity were opened to expose the organs. Blood was collected by direct cardiac puncture. After blood collection (if done), the animal was perfused with 1×PBS. Once perfusion was complete, organs were harvested carefully and divided into sections for histology or biological assay (Table 8 and 9). Sections used for assay were placed in an Eppendorf tube, then immediately submerged into liquid nitrogen. Sections saved for histology were placed in pre-labeled cassettes and submerged in 4% paraformaldehyde (PFA); after 24 hours, the PFA was replaced with 1×PBS and the cassettes were sent off for processing.

TABLE 9
Tissue harvest list for
401.4, ICM + IM
administration

| | Frozen | |
|---|---|---|
| Tissue | (−80° C.) | Histology |
| Brain | X | X |
| Spinal Cord | X | X |
| Liver | X | X |
| L TA | X | X |
| R TA | X | X |
| Quad | X | X |
| Heart | X | X |

TABLE 8
Tissue harvest list for
401.3, IV administration

| | Frozen | |
|---|---|---|
| Tissue | (−80° C.) | Histology |
| Brain | X | X |
| Heart | X | X |
| Liver | X | X |
| Spleen | X | X |
| R. Quad | X | X |
| L. TA | X | X |
| R. TA | X | X |
| Serum | X | |

Mitochondrial Isolation from Tissues

Mitochondrial isolation was performed as described in Preble et al. In summary, after preparation of homogenization buffer, fresh samples were homogenized using the gentleMACS™ Dissociator (Miltenyi Biotec). The homogenate was passed through a 40 μm filter followed by a 10 μm filter. The eluate was centrifuged at 9,000×g for 10 minutes at 4° C. Pellets were collected and resuspended in ELISA buffer for protein estimation, Immunoblot, and ELISA.

ELISA Quantification of Human Frataxin in Mitchondrial Fractions from Tissues

Human and mouse frataxin levels were analyzed in isolated mitochondrial fractions from mouse tissues using Human Frataxin ELISA Kit (ab176112) and Mouse Frataxin ELISA Kit (ab199078) according to the manufacturer's instructions.

### Histology

### Tissue Processing and H&E

At harvest, a portion of each tissue was fixed immediately in 4% parafomaldehyde (PBS 7.4) before paraffin-embedding and sectioning. Slides were dewaxed and re-hydrated using xylenes followed by an ethanol gradient. Hematoxylin and Eosin (H&E) staining was performed using a Leica auto-stainer at the University of Florida Molecular Pathology Core.

### Frataxin Immunofluorescence

Once slides were rehydrated, citrate antigen retrieval was performed in a steamer followed by streptavidin/biotin blocking (Vector Laboratories, SP-2002). An anti-mouse IgG (Vector Laboratories, MKB-2213-1) and serum block was also performed before application of the primary mouse anti-frataxin antibody (purified) at 1:300 overnight at 4° C. A biotinylated horse anti-mouse antibody (BA-2000) was applied 1:300 for 10 minutes at room temperature to tissue sections, then washed before application of the fluorescent Dylight 488 conjugated streptavidin (Vector Laboratories, SA-5488-1) at 1:200 for 10 minutes at room temperature. Finally, slides were counterstained with DAPI and treated for auto-fluorescence background according to the manufacturer's protocol (Vector Laboratories, SP-8400-15). Finally, the slides were mounted using Vectashield Vibrance mountant (Vector Laboratories, H-1700-10).

### Microscopy

Image acquisition was performed using the Keyence all-in-one microscope (BZ-X810). All H&E slides were scanned at 10× magnification with brightfield settings. To image frataxin, heart and quadricep from 401.3 and quadricep and tibialis anterior from 401.4 were scanned at 20×. All heart and skeletal muscle sections were scanned using the same settings, heart was scanned at high resolution and muscle with standard resolution. All fluorescent scans included the red channel for contrast and to determine background correction for determination of positive staining.

### Histopathology

For all groups, brain or spinal cord were examined carefully for immune infiltrates. Immunotoxicity in muscle was scored by the presence of necrotic fibers, mineralization, vacuolization, fibrosis, and presence of centralized nuclei on a scale ranging from none to severe (0-3). In IV administered animals, livers were scored by number and size of immune cell infiltrates, per field, on a scale ranging from none to severe (0-3).

### Image Analysis

All images were edited using the same methods and settings. Red and green channels were merged, and background removed using FIJI (Image JT). The signal-to-noise was low: therefore, quantitative analysis could not be performed using traditional thresholding methods. Stained tissues from treated animals were compared with stained tissues from sham-injected or untreated animals.

### Statistical/Analysis

All data were expressed as an average ± standard error or standard deviation, as indicated. Statistical analyses were performed with GraphPad Prism 8 (GraphPad Software).

### Results

Addition of a 5′-Untranslated Region to Frataxin Expressing Plasmids Reduces Toxicity while Enhancing Transduction and Expression of Frataxin Protein In Vitro

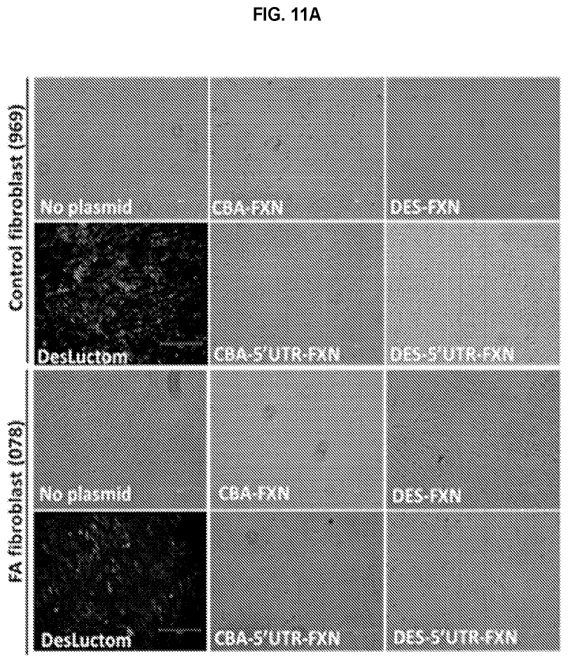

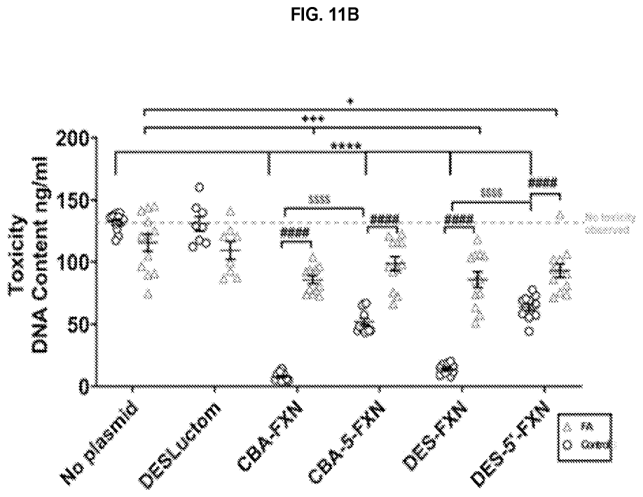

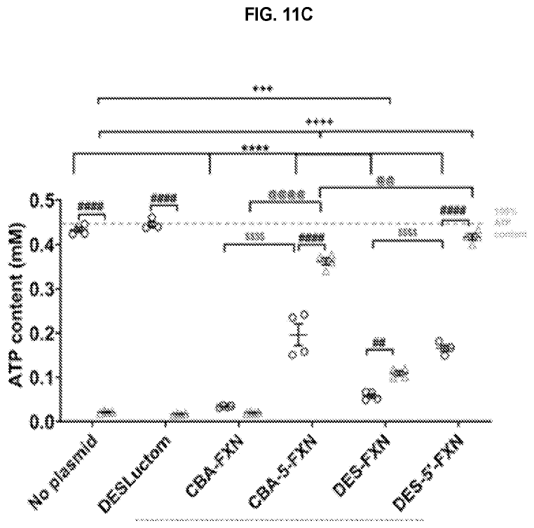

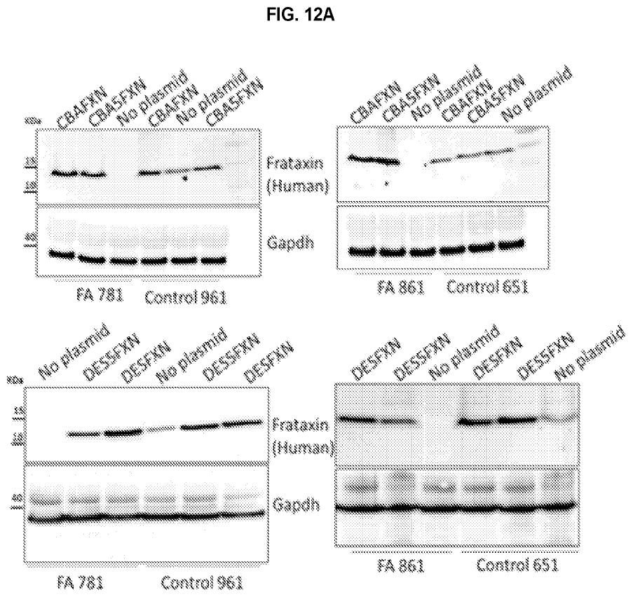

Fibroblasts from healthy individuals (control) and Friedreich's ataxia patients (TA) were treated with plasmid constructs expressing FXN under the control of a CBA or DES promoter with or without a 5′UTR (Table 1). Cells were imaged in a 24-well plate for visualization of cell confluency after transfection with the different constructs (FIGS. 11A-B). To quantify cell viability, DNA content was measured by CyQUANT Proliferation Assay (FIG. 11C). Untreated cells and cells transfected with a dual reporter plasmid (luciferase-furin2a-tdTomato) under the control of a DES promoter were used as negative and transfection-control, respectively. The blue line in FIG. 11C represents the value at which no toxicity was observed, as determined by the DNA content in normal untreated fibroblasts. All FXN expressing plasmids showed some level of toxicity in both normal and FA fibroblasts. In patient fibroblasts, this level of toxicity remained relatively constant across all FXN plasmid transfections. However, in control fibroblast cell lines, higher DNA content was observed in cells treated with plasmids containing the 5′UTR suggesting this region regulates FXN expression and reduces cellular toxicity.

To determine the effect of plasmid transfection on ATP levels, mitochondria were isolated from untreated and plasmid transfected fibroblasts from healthy and FA affected (FIG. 11D). Overall, ATP content was higher in fibroblast cultures treated with plasmids containing the 5′UTR compared to plasmids without a 5′UTR. In addition, plasmids containing the 5′UTR significantly increased mitochondrial ATP content in disease fibroblasts compared to untreated FA fibroblasts, indicating FXN overexpression restored ATP content in diseased cell lines. 5′UTR regulated FXN expression decreased ATP content in normal fibroblasts compared to untreated healthy fibroblasts. This suggests high overexpression of FXN in normal fibroblasts leads to toxicity. This data is consistent with the results from toxicity assay.

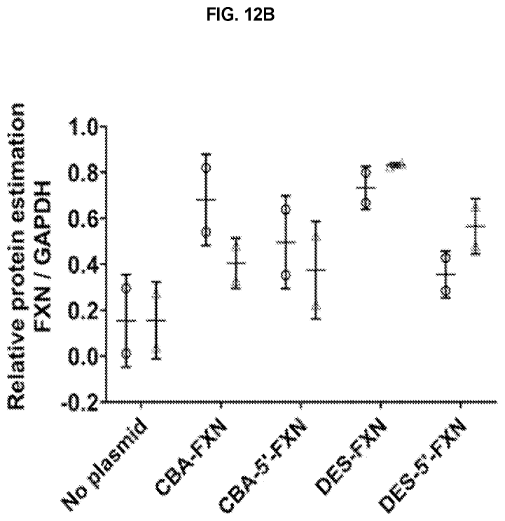

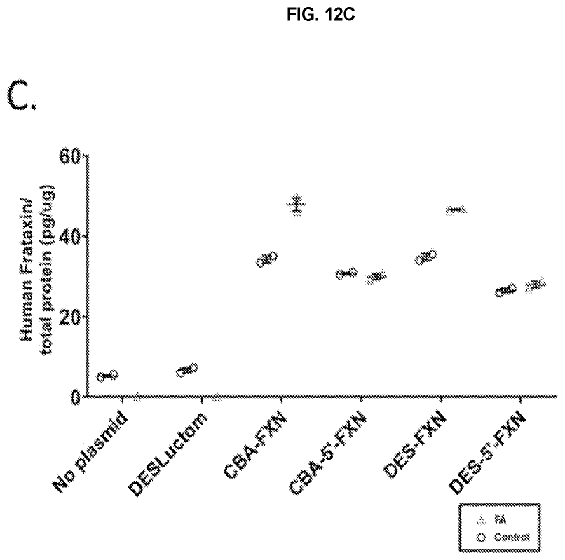

Western blot (FIGS. 12A-B) and ELISA (FIG. 12C) assays were conducted on cell lysates of transfected control and diseased fibroblasts to detect human FXN. In both assays, all four FXN expressing plasmids successfully transduced cells. DES-5′UTR-FXN appears to have lower FXN expression compared to DES-FN by Western blot in FA fibroblasts. These results were confirmed and quantified by ELISA showing 60% higher expression in DES-5′UTR-FXN compared to DES-FXN. ELISA showed similar results in control healthy fibroblasts, but the fold difference was negligible in comparison to diseased fibroblasts. Overall, across all cell lines, ELISA quantified expression levels were higher in cells transfected with plasmids lacking a 5′UTR (CBA-FXN and DES-FN) compared to plasmids with a 5′UTR (CBA-5′UTR-FXN and DES-5′UTR-FXN). This data suggests the 5′UTR element can sufficiently control the overexpression of FXN, leading to reduced toxicity.

### Comparison of a 5′-Untranslated Region and 3′-Untranslated Regions of Frataxin Plasmids In Vitro

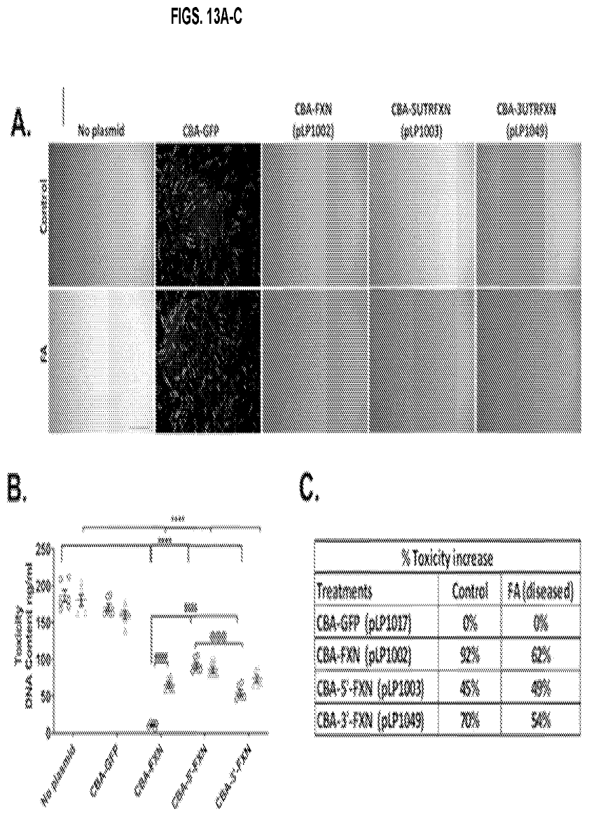

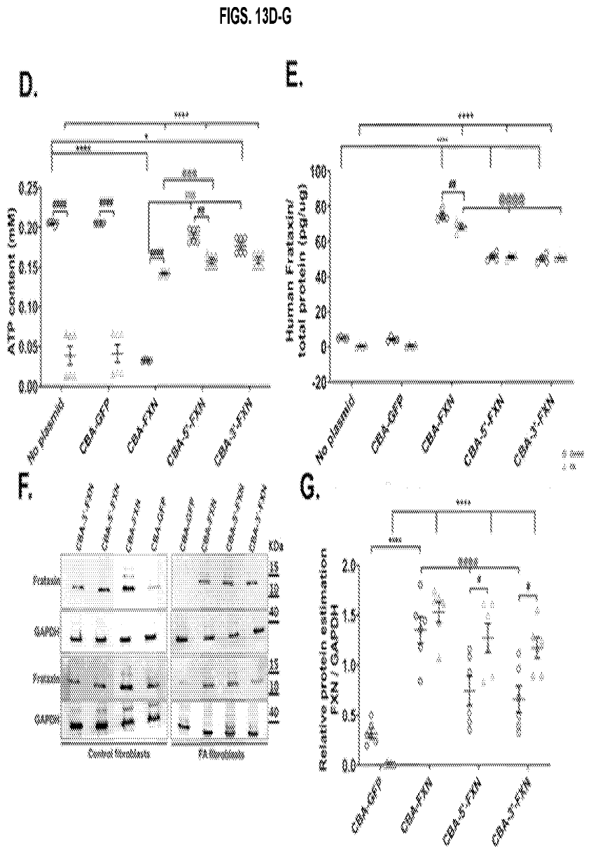

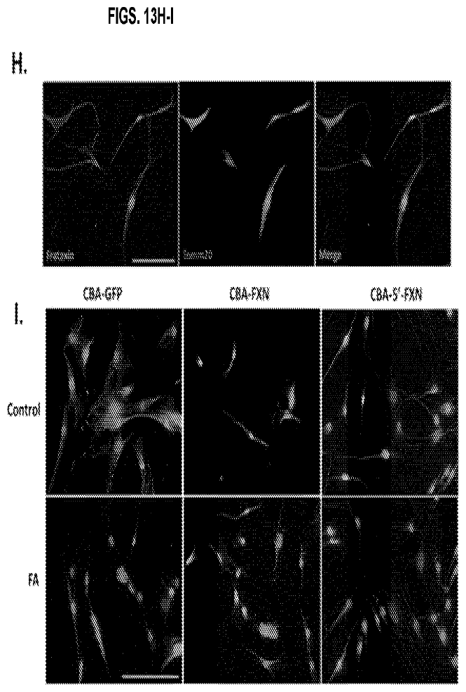

Fibroblasts from healthy (control) and FA patients were transfected with 5 pig of plasmid expressing FXN with or without a UTR under the control of CBA promoter (Table 2). Cells that were not transfected (no plasmid) and cells transfected with CBA-GFP were used as negative and transfection control, respectively. Cells were imaged in a 24-well plate for visualization of cell confluency after transfection of constructs (FIG. 13A). Cell viability was measured after transfection by CyQUANT assay (FIG. 13B). Toxicity analyses revealed CBA-FXN decreased cell viability in control fibroblasts when compared to CBA-5′-FXN. However, FA fibroblasts do not show the same distribution of toxicity (FIG. 13C). Similarly, ATP content was measured in non- and transfected cells (FIG. 13D). Detection of frataxin overexpression by ELISA was ˜16 times higher in CBA-FXN transfected control fibroblasts above endogenous frataxin levels. CBA-5′-FXN and CBA-3′-FXN were ˜10 times higher in expression when compared to endogenous frataxin expression (CBA-GFP). Densitometric analysis was performed after western blot directed against frataxin and GAPDH (FIG. 13E-13G). Immunocytochemistry detection of frataxin and tomm20 confirmed co-localization of frataxin in mitochondria (FIG. 13H) (19) and staining of control and diseased cells in under each condition was reflective of protein expression (FIG. 13I). Titration of plasmid content was performed to reduce toxicity in vitro (FIGS. 14A-B).

### Biodistribution and No Associated Toxicity Following In Vivo Administration of AAV8TM-DES-5′UTR-FXN

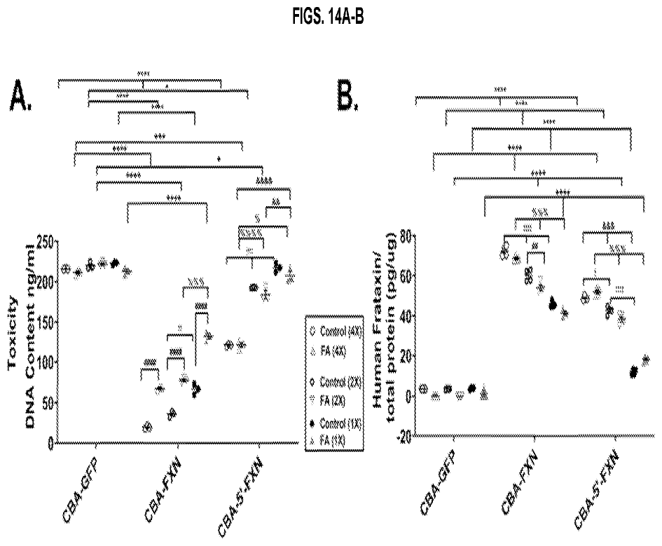

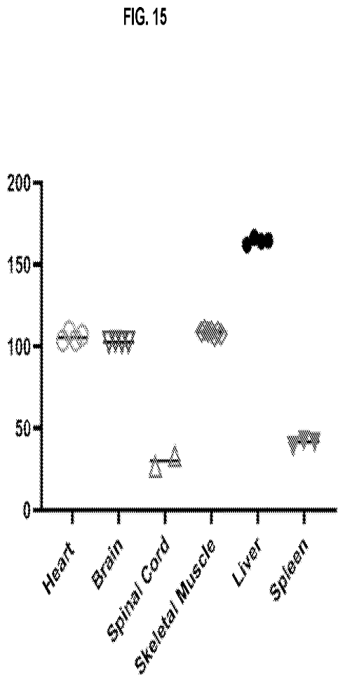

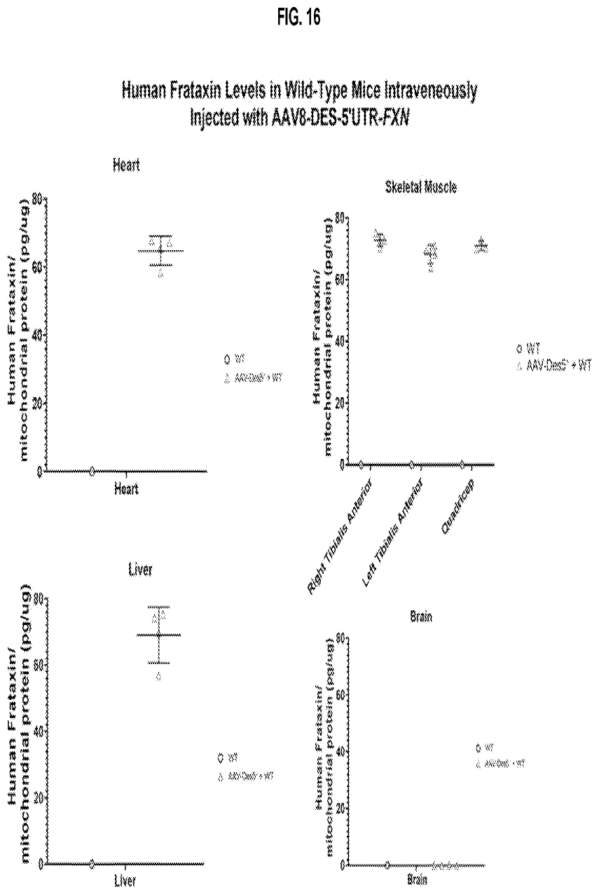

Frataxin levels were measured in the heart, brain, spinal cord, skeletal muscle, liver, and spleen of wild type mice. Normal ranges of mouse frataxin protein was determined after ELISA assay in un-injected animals (FIG. 15). A separate set of wild type mice received an intravenous injection of AAV8TM-DES-5′UTR-FXN at 9 weeks of age to determine potential toxicity resulting from frataxin overexpression in normal animals. Quantification of human frataxin (ELISA) in heart, skeletal muscle, liver, and brain of normal mice following AAV administration results in supra-physiologic levels of FXN expression (FIG. 16). Hematoxylin and eosin staining was conducted to determine if inflammation or toxicity was evident in heart, skeletal muscle, liver, and brain. The staining demonstrated no- to negligible toxicity in the tissues of the injected animals.

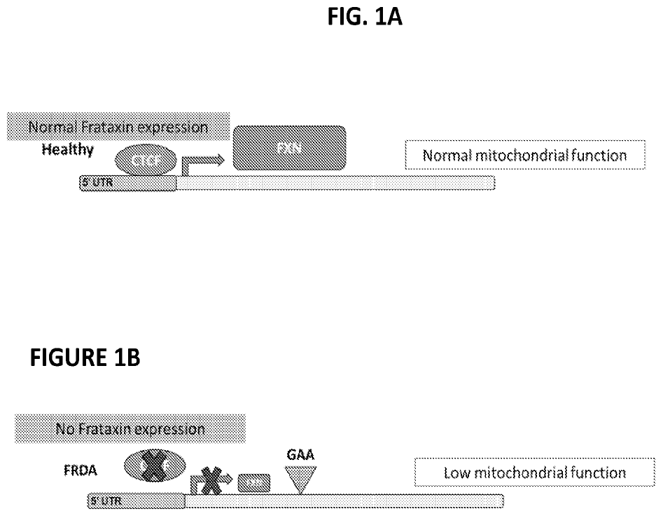

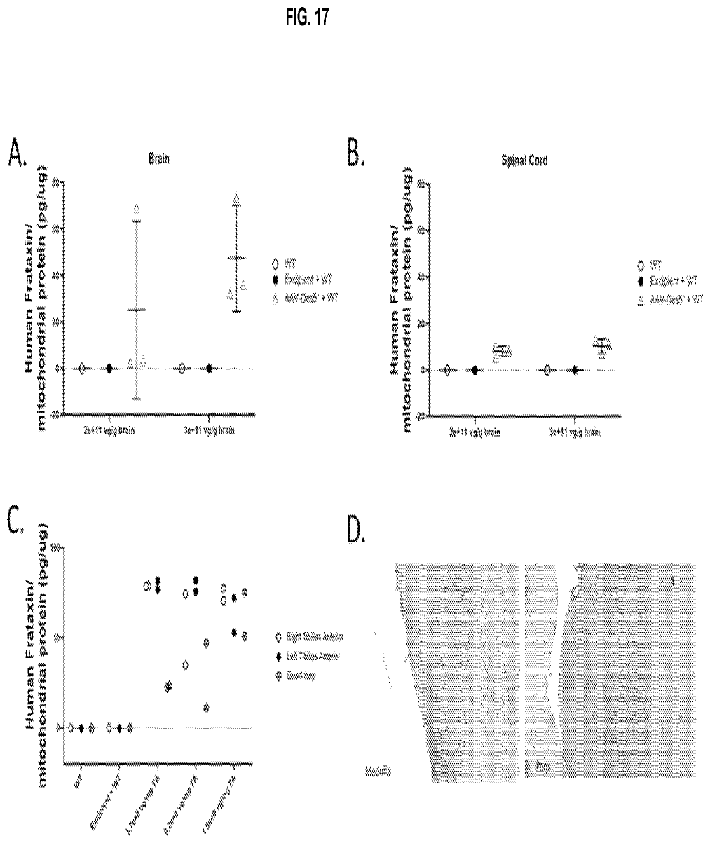

Following AAV delivery via ICM, brains of wild type mice were assessed for human frataxin (ELISA). Detection of human frataxin was observed in the brain and spinal cord at each dose. Unexpectedly, detection of frataxin was not observed in a subset of the animals (2/3) (FIG. 17A-B). The same animals also received a dose via direct intramuscular injection in the right and left tibialis anterior (TA) muscle at three ascending doses. Assessment of frataxin in TA lysates (ELISA) following intramuscular administration revealed significant expression; however, detection of frataxin was also observed in the quadriceps. This suggests intramuscular injection (TA) may have resulted in leakage to the circulatory system or an alternative mechanism whereby the quadriceps exhibit frataxin (FIG. 1′C). Representative images and histochemical analysis of brain regions demonstrate positive detection of frataxin (FIG. 171).

### In Vitro FAV Toxicity Human Fibroblasts

These results suggest inclusion of the frataxin 5′UTR with frataxin results in lower toxicity as compared to the frataxin ORF alone. Presence of the 5′UTR positively affects or maintains desired ATP content in fibroblasts, which further supports reduced risk for toxicity. Results indicate frataxin overexpression, without 5′UTR control, is highly toxic to normal fibroblast cell lines. Inclusion of the 5′UTR also led to more normalized mitochondrial ATP content in transfected cell lines. 5′UTR frataxin expression was lower for both DES and CBA promoter driven cassettes when compared to non-5′UTR containing frataxin cassettes. These results show that inclusion of the frataxin 5′UTR with frataxin in the cassette significantly reduces FXN overexpression related toxicity in vitro (normal and patient fibroblasts). Administration of frataxin with the 5′TR element also improves mitochondrial respiration of primary disease-associated tissues. Statistical analysis reveals significant elevation of frataxin following transfection with CBA-FXN (˜16×) or CBA-5′-FXN (10×) when transfected with 5 μg of plasmid. At lower DNA transfection levels, the CBA-5′-FXN no longer exhibits toxicity within control fibroblasts, while CBA-FXN still results in loss of cell viability. Furthermore, these results show proper trafficking of frataxin via co-localization of frataxin and mitochondria following transfection.

### In Vivo FXN Toxicity: Intravenous Administration in Normal Wild-Type Mice

The objective of this study was to understand the toxicity following intravenous injection (5E+13 vg/kg) in wild type mice. Upon histological H&E examination, no obvious toxicity was found in heart, liver, skeletal muscle, brain, or spinal cord. Detection of human frataxin by ELISA revealed a significant increase in expression in peripheral tissues. Results of this study show that AAV-Des5′ at 5E+13 vg/kg does not induce toxicity in examined tissues where overexpression of human frataxin was observed.

### In Vivo FXN Toxicity: ICM+IM Administration in Normal Wild-Type Mice

The objective of this study was to determine whether a toxicity-dose relationship is observed following dual routes of administration (ICM+IM) of AAV-Des5′. Upon histological examination, no obvious toxicity was observed in brain or skeletal muscle. IM injection (TA) also resulted in detection of frataxin expression in the quadriceps. ICM. AAV administration at 3E+11 vg/g brain resulted in the highest frataxin expression and may be attributed to higher dose. Results of this study support the hypothesis that AAV-Des5′ can express frataxin in targeted tissues without toxicity.

Since freshly isolated tissues are required for ATP content measurement and variability in frataxin expression is observed within the same cohort, human FXN 65 pg/ug Vas expressed; normal mouse frataxin 102 pg/ug in heart human FXN 60 pg/ug; normal mouse frataxin 109 pg/ug in skeletal muscles, human FXN 36 pg/ug; normal mouse frataxin 103 pg/ug in brain and human FXN 9.2 pg/ug; normal mouse frataxin 26.6 pg/ug in spinal cord. [Please explain the above experiment in more detail.]

No AAV5′Des-induced toxicity was observed in AAV injected animals which supports limited potential for immunogenic response to the AAV vector in the context of FA gene therapy. Table 10 outlines frataxin expression in heart, skeletal muscle, and liver in the MCK-Cre mouse model of Friedreich's Ataxia disease compared to Bamboo Therapeutics (See International Patent Application Publication No. WO2017077451) following AAV delivery. Bamboo Therapeutics used AAV2i8-HA-FXN at a dose of 1×10¹³ vg/kg intravenously in three-week-old MCK Fxn−/− mice.

TABLE 10
Comparison of AAV-mediated human frataxin expression in
MCK mice following IV delivery.

| | pg/ug = ng/mg | | |
|---|---|---|---|
| | Lacerta data | | |
| | Frataxin in tissue (pg/ug proteins) | | |
| | Heart | Sk. Muscle | Liver |
| Treated n = 4 | 64 +/− 2.1 | 60 +/− 0.9 | 69 +/− 4.2 |
| | A61K 48/00 (2006.01) - BAMBOO Therapeutics | | |
| | Frataxin in tissue (ng/mg proteins) | | |
| | Heart | Sk. Muscle | Liver |
| Treated n = 4 | 38 +/− 1.99 | 4.57 +/− 0.4 | 0.07 +/− 0.01 |

### Effects of Intron Placement in AAV Construct

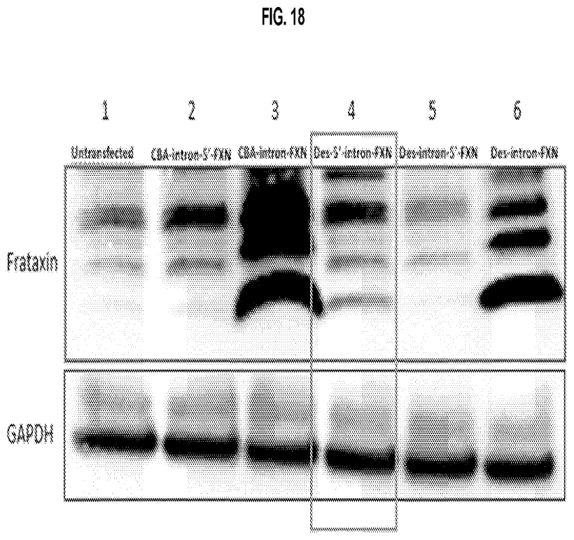

As shown in FIG. 18, the order of the elements in the AAV construct impacts FXN expression. Constructs that do not include a 5′UTR results in highly significant expression (lanes 3 and 6, FIG. 18) in C2C12 mouse myoblasts. Inclusion of the 5′ UTR between the intron and FXN results in low FNX expression (lane 5, FIG. 18) in C2C12 mouse myoblasts. However, inclusion of the 5′UTR, an intron and FXN, in that order, results in desired FXN expression levels.

In summary, toxicity was observed in a dose-dependent manner, in normal, control or FA patient fibroblast cell lines, at supraphysiologic FXN expression levels. No toxicity was observed in normal mice following delivery of AAV-5′UTR-FXN in the brain, spinal cord or skeletal muscle. Overexpression of 5′UTR-FXN does not result in obvious toxicity in vivo but loss of cell viability is detected in vitro at highly significant levels of FXN overexpression. Regulation of FXN expression by inclusion of the 5′UTR region reduces the potential for overexpression-induced cellular toxicity.

### Example 8

### Protein Expression and Quantification for all Plasmid Constructs

Human FXN promoter-intron-codon optimized frataxin will be cloned in pdsAAV-CB-EGFP (MK225672) which contains a chicken beta actin promoter (CBA) AND CMV enhancer. Successful cloning will be confirmed through Sanger sequencing. After confirmation, the plasmids will be overexpressed according manufacturer's protocol with Trans-IT (Mirusbio, Madison Wis.). The following constructs will be tested:

CBA-5UTR-INTRON-FXN: the construct containing 5UTR frataxin upstream of sv40 INTRON with CBA promoter

CBA FXN: the construct containing frataxin with CBA promoter

CBA-INTRON-5 ITR-FXN: THE CONSTRUCT containing 5UTR frataxin downstream of sv40 INTRON with CBA promoter

CBA-hFXNpromoter-FXN: the construct containing endogenous human frataxin promoter and codon optimized frataxin

via transient transfection in C2C12 murine myoblasts cell lines.

Following transfections of these cell lines, the cell pellets are collected, and protein isolated by RIPA buffer. A 16% tricine SDS PAGE gel will be run to separate the proteins. After SDS PAGE, the i-blot (Thermofisher Scientific) module will be used to transfer the separated proteins onto a nitrocellulose membrane. The nitrocellulose membrane will be blocked with 5%0 milk in TBST buffer for 2 hours and then probed with primary antibodies and HRP-conjugated secondary antibodies respectively. The western blot is then visualized in i-Bright device after incubation with chemiluminescence solution (Millipore) for 5 minutes.

Overexpressed human frataxin protein will be probed with specific antibodies α-Frataxin antibodies (Abcam). Gapdh (Cell Signaling technologies) is the loading control to confirm equal amount of protein loading in each lane. Successful, modulated expression of frataxin is expected from the construct including a 5′ UTR FXN an sv40 INTRON and a CBA promoter, in that order.

### RNA Transcripts for all Plasmid Constructs

Human FXN promoter-intron-codon optimized frataxin will be cloned in pdsAAV-CB-EGFP (MK225672) which contains a chicken beta actin promoter (CBA) AND CMV enhancer. Successful cloning will be confirmed through Sanger sequencing. After confirmation, the plasmids will be overexpressed according manufacturer's protocol with Trans-IT (Mirusbio·Madison Wis.). The following constructs will be tested:

CBA-5UTR-INTRON-FXN: the construct containing 5UTR frataxin upstream of sv40 INTRON with CBA promoter

CBA FXN: the construct containing frataxin with CBA promoter

CBA-INTRON-5UTR FXN: THE CONSTRUCT containing 5UTR frataxin downstream of sv40 INTRON with CBA promoter

CBA-hFXNpromoter-FXN: the construct containing endogenous human frataxin promoter and codon optimized frataxin

via transient transfection in C2C12 murine myoblasts cell lines.

Following transfections, the cells will be collected to isolate RNA with an RNA isolation kit (Thermofisher Scientific). cDNA will generated from these RNA and qPCR will be conducted to validate the human frataxin copies in each condition.

### Regulation of Protein Expression by Silencing the L2 Region

siRNaA will be designed to specifically target the L2 region of the 5′ UTR (SEQUENCE ID 33). C2C12 cells will co-transfected with the plasmids mentioned above and siRNA.

Following transfections of these cell lines, the cell pellets will be collected, and proteins isolated by RIPA buffer. A 16% tricine SDS PAGE gel will be run to separate the proteins. After SDS PAGE, an i-blot (Thermofisher Scientific) module will be used to transfer the separated proteins onto a nitrocellulose membrane. The nitrocellulose membrane will be blocked with 5% milk in TBST buffer for 2 hours and then probed with primary antibodies and HRP-conjugated secondary antibodies respectively. The western blot is then visualized in i-Bright device after incubation with chemiluminescence solution (Millipore) for 5 minutes.

Overexpressed human frataxin protein will be probed with specific antibodies α-Frataxin antibodies (Abeam). Gapdh (Cell Signaling technologies) is the loading control to confirm equal amount of protein loading in each lane. The results will indicate that siRNA targeted cells produce high levels of frataxin compared to cells without treatment of siRNA in the above-mentioned cell line. Also, the frataxin without the 5′ UTR expresses relatively more than frataxin with 5′UTR.

### Example 9

### Therapeutic Efficacy of AAV8TM-CBA-5′-FXN in the Cardiac Mouse Model of Friedreich's Ataxia

The following experiments will be performed to test the efficacy of regulated 5′UTR-FXN compared to unregulated (no 5′UTR) FXN. The cardiac-specific FXN KO (Fxnflox/mull::MCK-Cre (Jax: 029720)) mouse model has an approximate lifespan ˜9-10 weeks without therapeutic intervention. AAV8TM-CBA-5′-FXN, 5e13 vg/kg virus will be administered intravenously at post-natal day 0 (PND0) or 5 weeks of age; pre-symptomatic and moderate disease stage, respectively. Animals will undergo cardiac MR (11T) to determine cardiac function and morphometry at 9 weeks of age. The goal is to attenuate development of cardiac dysfunction following AAV8TM-CBA-5′-FXN delivery.

### Research Strategy

AAV8TM-CBA-5′-FXN viruses will be made at the Powell Gene Therapy Center, Vector Core at the University of Florida and titered for injections via digital drop PCR. Four week old Fxnflox/null::MCK-Cre mice will be injected with 5×1013 vg/kg dose. Recruitment of animals in each group will follow with a single bolus of test article via intravenous (IV) injection. Body weights will be recorded on a weekly basis. Twenty-eight days post-dose, MRI imaging will be conducted to observe clinically relevant cardiac endpoint in the cardiac mouse model. Left ventricular stroke volume, left ventricular ejection fraction, left ventricular shortening fraction and cardiae output will be measured (Segment software; Medviso). After cardiac imaging, the animals will be sacrificed, and necropsy will include collection of whole blood, brain, spinal cord, dorsal root ganglion, cerebrospinal fluid, heart, left and right quadriceps, left and right tibialis anterior (TA), liver and spleen. Freshly harvested tissues will be subjected to immediate mitochondrial isolation followed by ATP analysis (ATPlite Luminescence Assay. Perkin Elmer). A remaining piece of tissue will be subjected for histological analysis of toxicity, fibrosis, iron deposition and lipid droplets analysis. Mitochondrial will be isolated from frozen tissues for quantitation of human frataxin by ELISA assay (Abcam) and western blot. Blood serum will be collected for potentially clinically relevant assessments; GDF-15 serun levels and cardiac troponin I have been reported to increase in Fxn null mice. The plan of the study is elaborated in Table 11, For Table 12. E10.5 pregnant females will be ordered and PO (postnatal day 0) littermates will be injected with 5×1013 vg/kg dose through temporal vein injection. 4 weeks post injection, MRI imaging will be conducted to observe their phenotype as mentioned above. After that 8 weeks post injection, MRI imaging will be conducted to understand disease progression and therapeutic effect of the AAV8TM-CBA-5′-FXN

TABLE 11
Experimental design for gene therapy study in adult cardiac mouse model of FA

| | Mouse | | | Injection | | Age @ | | Study |
|---|---|---|---|---|---|---|---|---|
| Group | Strain | Treatment | Dose | Route | Gender | Inj. | n | Duration |
| 1 | B6/J | Excipient | 100 μL | IV | M | 4 wk | 10 | 28-days |
| 2 | MCK-FXN^{-/-} | Excipient | 100 μL | IV | M | 4 wk | 10 | 28-days |
| 3 | MCK-FXN^{-/-} | 8TM-CBA-FXN | 5.0e13 vg/kg | IV | M | 4 wk | 10 | 28-days |
| 4 | MCK-FXN^{-/-} | 8TM-CBA-5′-FXN | 5.0e13 vg/kg | IV | M | 4 wk | 10 | 28-days |

TABLE 12
Experiinetrtal design lot gene alerapy sirtcly in ileotiatal cardiac triouse model of FA

| | Mouse | | | Injection | | Age @ | | Study |
|---|---|---|---|---|---|---|---|---|
| Group | Strain | Treatment | Dose | Route | Gender | Inj. | n | Duration |
| 1 | B6/J | Excipient | 100 μL | IV | M | P0 | 10 | 56-days |
| 2 | MCK-FXN^{-/-} | Excipient | 100 μL | IV | M | P0 | 10 | 56-days |
| 3 | MCK-FXN^{-/-} | 8TM-CBA-FXN | 5.0e13 vg/kg | IV | M | P0 | 10 | 56-days |
| 4 | MCK-FXN^{-/-} | 8TM-CBA-5′-FXN | 5.0e13 vg/kg | IV | M | P0 | 10 | 56-days |

Based on preliminary data, disease progression is expected to be halted in the cardiac model in the groups. Heart weight in the injected diseased animals will be close to normal. An increase in the ATP levels in the tissues is also expected. Histology indices should reveal decreased fibrosis, iron deposition and lipid droplets in animals receiving AAV8TM-CBA-5′-FXN. Comparison of AAV8TM-CBA-FXN and AAV8TM-CBA-5′-FXN will elucidate whether excessive frataxin overexpression is toxic in animals.

### Therapeutic Efficacy of AAV8TM-CBA-5′-FXN in the Neuronal Mouse Model of Friedreich's Ataxia

To test the efficacy of AAV8TM-CBA-5′-FXN in the CNS, 4 or 12 week old Fxnflox/null::PV-Cre (Sax: 029721) animals will receive vector delivery in the cerebrospinal fluid via intracisterna magna (ICM) injection at a dose of 1.5e11 vg/g of brain. Animals will undergo monthly behavioral assessments starting at 8 weeks-20 weeks of age. The goal is to attenuate development of neuronal and neuromuscular dysfunction following AAV8TM-CBA-5′-FXN delivery.

### Research Strategy

4-5 week old Fxnflox/null::PV-Cre mice will be recruited for these studies. Fxnflox (floxed exon 2) mice have a CRISPR/Cas9-generated, Cre-conditional frataxin allele which will be used as a control for the experiment. Mice in groups 1-4 will receive a single bolus of excipient or test article (1.5e11 vg/g brain) via intra-cisterna magna (ICM) injection. Body weights will be recorded on a weekly basis. Behavioral tests using Rotarod, neuroscore, wirehangs and forelimb grip strength tests will be evaluated at 4, 8, 10, 12, 6, 18 and 20 weeks post dose as described in Table 3 and 12, 14, 16, 20 weeks post dose as described (Groups 5-8) [14, 15]. Twenty weeks post-dose, necropsy will include collection of whole blood, brain, spinal cord, dorsal root ganglion, cerebrospinal fluid, heart, left and right quadriceps, left and right tibialis anterior (TA), liver and spleen. Freshly-isolated mitochondria from key tissues will be subjected to ATP analysis. Remaining portions of tissue will be immediately frozen in liquid nitrogen or fixed (4% PFA) for histological analysis (Toxicity-GFAP staining; calbindin staining-rescue of Purkinje neurons and succinate dehydrogenase A-mitochondrial complex II). Frozen tissues will be subjected to mitochondrial isolation and subsequent molecular analysis for quantitation of human FXN by ELISA (Abcam) and western blot. The plan of the study is elaborated in Table 13.

TABLE 13
Experimental design for gene therapy study in neurona mouse mode of FA

| | Mouse | | | Injection | | Age @ | | Study |
|---|---|---|---|---|---|---|---|---|
| Group | Strain | Treatment | Dose | Method | Gender | Inj. | n | Duration |
| 1 | Fxnflox | Excipient | 100 μL | ICM | M | 4 wk | 10 | 20 wk |
| | (floxed exon 2) | | | | | | | |
| 2 | Fxnflox/null:: | Excipient | 100 μL | ICM | M | 4 wk | 10 | 20 wk |
| | PV-Cre | | | | | | | |
| 3 | Fxnflox/null:: | AAV8TM- | 1.5e11 vg/kg | ICM | M | 4 wk | 10 | 20 wk |
| | PV-Cre | CBA-FXN | brain | | | | | |
| 4 | Fxnflox/null:: | AAV8TM- | 1.5e11 vg/kg | ICM | M | 4 wk | 10 | 20 wk |
| | PV-Cre | CBA-5′-FXN | brain | | | | | |
| 5 | Fxnflox | Excipient | 100 μL | ICM | M | 4 wk | 10 | 20 wk |
| | (floxed exon 2) | | | | | | | |
| 6 | Fxnflox/null:: | Excipient | 100 μL | ICM | M | 4 wk | 10 | 20 wk |
| | PV-Cre | | | | | | | |
| 7 | Fxnflox/null:: | AAV8TM- | 1.5e11 vg/kg | ICM | M | 4 wk | 10 | 20 wk |
| | PV-Cre | CBA-FXN | brain | | | | | |
| 8 | Fxnflox/null:: | AAV8TM- | 1.5e11 vg/kg | ICM | M | 4 wk | 10 | 20 wk |
| | PV-Cre | CBA-5′-FXN | brain | | | | | |

These experiments will determine biodistribution and therapeutic impact following CSF delivery of AAV8TM-5′-FXN. Our biodistribution studies suggest this dose range will provide robust transduction in target cell populations in the CNS. This will result in attenuation or prevention of ataxia development in AAV8TM-Fxnflox/null::PV-Cre animals. Toxicity is not expected in the brain, dorsal root ganglia, or spinal cord. ATP content (biochemical) and Succinate dehydrogenase A (IHC) will increase in AAV8TM-CBA-5′-FXN treated Fxnflox/null::PV-Cre animals.

1. A nucleic acid construct comprising:
a nucleic acid sequence comprising a human frataxin 5′ untranslated region (5′UTR FXN); and
a nucleic acid sequence encoding human frataxin (FXN), wherein the nucleic acid sequence encoding human FXN has at least 70% sequence identity to SEQ ID NO: 1.

2. The nucleic acid construct of claim 1, wherein the 5′UTR FXN has at least 85% sequence identity to SEQ ID NO: 2.

3. The nucleic acid construct of claim 1, wherein the nucleic acid sequence encoding human FXN is codon-optimized.

4. The nucleic acid construct of claim 1, wherein the 5′UTR FXN comprises SEQ ID NO: 2.

5. The nucleic acid construct of claim 1, wherein the 5′UTR FXN is located upstream of the nucleic acid sequence encoding human FXN.

6. The nucleic acid construct of claim 1, further comprising an intron, wherein the intron is positioned downstream of the 5′UTR FXN and upstream of the nucleic acid encoding human FXN.

7. The nucleic acid construct of claim 1, wherein the 5′UTR FXN comprises a CCCTC-binding factor (CTCF) binding site.

8. (canceled)

9. The nucleic acid construct of claim 1, further comprising a nucleic acid sequence comprising an RNA polymerase II promoter.

10. A nucleic acid construct comprising, in the following order:
(a) a nucleic acid sequence comprising an RNA polymerase II promoter;
(b) a nucleic acid sequence comprising a 5′UTR FXN; and
(c) a nucleic acid sequence encoding human FXN,
wherein the RNA polymerase II promoter is not a frataxin promoter, and wherein the RNA polymerase II promoter is operably linked to the 5′UTR FXN and the nucleic acid sequence encoding a human FXN.

11. The nucleic acid construct of claim 10, wherein the nucleic acid sequence encoding human FXN has at least 70% sequence identity to SEQ ID NO: 1.

12. (canceled)

13. The nucleic acid construct of claim 1, wherein the nucleic acid construct comprises, in the following order:
(a) a nucleic acid sequence comprising RNA polymerase II promoter;
(b) a nucleic acid sequence comprising a 5′UTR FXN; and
(c) a nucleic acid sequence encoding human FXN,
wherein the nucleic acid sequence encoding human FXN has at least 85% sequence identity to SEQ ID NO: 1, and wherein the RNA polymerase II promoter is operably linked to the 5′UTR FXN and the nucleic acid sequence encoding a human FXN.

14. A nucleic acid construct comprising, in the following order:
(a) a nucleic acid sequence comprising an RNA polymerase II promoter;
(b) a nucleic acid sequence comprising a 5′UTR FXN;
(c) an intron; and
(d) a nucleic acid sequence encoding human FXN,
wherein the RNA polymerase II promoter is operably linked to the 5′UTR FXN and the nucleic acid sequence encoding a human FXN.

15. The nucleic acid construct of claim 14, wherein the nucleic acid sequence encoding human FXN has at least 70% sequence identity to SEQ ID NO: 1.

16. (canceled)

17. The nucleic acid construct of claim 1, wherein the nucleic acid construct comprises, in the following order:
(a) a nucleic acid sequence comprising RNA polymerase II promoter;
(b) a nucleic acid sequence comprising a 5′UTR FXN;
(c) an intron; and
(d) a nucleic acid sequence encoding human FXN,
wherein the nucleic acid sequence encoding human FX has at least 85% sequence identity to SEQ ID NO: 1, and wherein the RNA polymerase II promoter is operably linked to the 5′UTR FXN and the nucleic acid sequence encoding a human FXN.

18. The nucleic acid construct of claim 9, wherein the RNA polymerase II promoter is a desmin promoter or a CBA promoter

19. (canceled)

20. (canceled)

21. (canceled)

22. The nucleic acid construct of claim 1, further comprising a pair of inverted terminal repeats (ITR), wherein the nucleic acid construct is flanked on each said by an ITR.

23. A recombinant viral vector comprising the nucleic acid construct of claim 1.

24. The recombinant viral vector of claim 23, wherein the vector is an adeno-associated viral (AAV) vector.

25. The recombinant AAV vector of claim 24, wherein the AAV vector is selected from the group consisting of: AAV1 serotype vectors, AAV2 serotype vectors, AAV3 serotype vectors, AAV4 serotype vectors, AAV5 serotype vectors, AAV6 serotype vectors, AAV7 serotype vectors, AAV8 serotype vectors, AAV9 serotype vectors, AAV Rh74 serotype vectors, and combinations thereof.

26. (canceled)

27. (canceled)

28. (canceled)

29. A nucleic acid that comprises the recombinant AAV vector of claim 24.

30. The nucleic acid of claim 29, wherein the nucleic acid is a plasmid.

31. A recombinant AAV particle comprising the recombinant viral vector of claim 23.

32. A pharmaceutical composition comprising the particle or plurality of particles of claim 31.

33. (canceled)

34. A genetically modified cell comprising the nucleic acid construct of claim 1.

35. The genetically modified cell of claim 34, wherein the genetically modified cell is selected from the group consisting of: a human stem cell, a human neuron, a human cardiomyocyte, a human smooth muscle myocyte, a human skeletal myocyte, and a human hepatocyte.

36. A method of treating a patient with Friedreich's Ataxia (FA), the method comprising: administering a therapeutically effective amount of the recombinant AAV particle of claim 31 to the patient.

37. A method of modulating expression of FXN in a human cell, the method comprising, introducing into the human cell, the recombinant AAV vector of claim 24.

38. A method of modulating expression of FXN in a human cell, the method comprising, introducing into the human cell, a nucleic acid of claim 29.

39. A method of increasing adenosine triphosphate (ATP) concentration in a human cell of a subject with FA, the method comprising administering a therapeutically effective amount of the recombinant AAV particle of claim 31 to the patient.

40. A method of increasing ATP concentration in a human cell of a subject with FA, the method comprising administering a therapeutically effective amount of the recombinant AAV particle of claim 31 to the patient.

41. The method of claim 38, wherein the human cell is selected from the group consisting of: a neuron, a cardiomyocyte, a smooth muscle myocyte, a skeletal myocyte, and a hepatocyte.


SEQUENCE LISTING (63 sequences)

SEQ ID NO: 1 - DNA, 633 bp, Artificial sequence
  Synthetic construct
atgtggacattggggcggagggcagtggcgggtcttcttgcgtctcccagcccagcacaggcacaaacattgactagagttccccggccagcggagttggcccctctctgtggacggcggggactgcggacggatatagacgccacctgcacacctcgaagagctagttcaaatcagcggggcctcaatcaaatctggaacgttaagaagcagagtgtgtaccttatgaacttgagaaaaagcggaaccctcggccacccagggtcattggatgaaacaacctatgagaggcttgcggaagagacattggatagcttggccgaattctttgaagaccttgccgacaaaccctatacatttgaggattacgatgtctccttcggctctggtgtcctgactgtgaagttggggggcgacctcggaacgtacgtaataaataagcagactccgaataaacaaatttggttgtcctcaccaagtagcggccccaagcggtatgattggactgggaagaactgggtatactcccacgacggcgttagcctgcacgaactgttggcagccgagcttacaaaagctttgaagacaaaactggacctcagttctttggcctattcagggaaagacgcatag

SEQ ID NO: 2 - DNA, 221 bp, Homo sapiens
cagtctcccttgggtcaggggtcctggttgcactccgtgctttgcacaaagcaggctctccatttttgttaaatgcacgaatagtgctaagctgggaagttcttcctgaggtctaacctctagctgctcccccacagaagagtgcctgcggccagtggccaccaggggtcgccgcagcacccagcgctggagggcggagcgggcggcagacccggagcagc

SEQ ID NO: 3 - DNA, 20 bp, Homo sapiens
agtggccaccaggggtcgcc

SEQ ID NO: 4 - DNA, 611 bp, Homo sapiens
gatcttaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgagataaccagggctgaaagaggcccgcctgggggctggagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggcc

SEQ ID NO: 5 - DNA, 515 bp, Homo sapiens
ccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgatatcactagtgcca

SEQ ID NO: 6 - DNA, 2324 bp, Artificial sequence
  Synthetic construct
ctgcgcgctcgctcgctcactgaggccgcccgggcaaagcccgggcgtcgggcgacctttggtcgcccggcctcagtgagcgagcgagcgcgcagagagggagtggaattcacgcgtggtacgatctgaattcggtacaattcacgcgtggtaccgatcttaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgagataaccagggctgaaagaggcccgcctgggggctggagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgtccgccgcctcctccgtgcgcccgccagcctcgcccgcgccgtcaccgtgaggcactgggcaggtaagtatcaaagtatcaaggttacaagacaggtttaaggagaccaatagaaactgggcttgtcgagacagagaagactcttgcgtttctgataggcacctattggtcttactgacatccactttgcctttctctccacaggctagcctcgagaattcacgcgtggtacctctagagtcgaccagtctcccttgggtcaggggtcctggttgcactccgtgctttgcacaaagcaggctctccatttttgttaaatgcacgaatagtgctaagctgggaagttcttcctgaggtctaacctctagctgctcccccacagaagagtgcctgcggccagtggccaccaggggtcgccgcagcacccagcgctggagggcggagcgggcggcagacccggagcagcgccaccatgtggacattggggcggagggcagtggcgggtcttcttgcgtctcccagcccagcacaggcacaaacattgactagagttccccggccagcggagttggcccctctctgtggacggcggggactgcggacggatatagacgccacctgcacacctcgaagagctagttcaaatcagcggggcctcaatcaaatctggaacgttaagaagcagagtgtgtaccttatgaacttgagaaaaagcggaaccctcggccacccagggtcattggatgaaacaacctatgagaggcttgcggaagagacattggatagcttggccgaattctttgaagaccttgccgacaaaccctatacatttgaggattacgatgtctccttcggctctggtgtcctgactgtgaagttggggggcgacctcggaacgtacgtaataaataagcagactccgaataaacaaatttggttgtcctcaccaagtagcggccccaagcggtatgattggactgggaagaactgggtatactcccacgacggcgttagcctgcacgaactgttggcagccgagcttacaaaagctttgaagacaaaactggacctcagttctttggcctattcagggaaagacgcatagtagtctagagatatcgcggccgcttcggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 7 - DNA, 5791 bp, Artificial sequence
  Synthetic construct
ctgcgcgctcgctcgctcactgaggccgcccgggcaaagcccgggcgtcgggcgacctttggtcgcccggcctcagtgagcgagcgagcgcgcagagagggagtggaattcacgcgtggtacgatctgaattcggtacaattcacgcgtggtaccgatcttaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgagataaccagggctgaaagaggcccgcctgggggctggagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgtccgccgcctcctccgtgcgcccgccagcctcgcccgcgccgtcaccgtgaggcactgggcaggtaagtatcaaagtatcaaggttacaagacaggtttaaggagaccaatagaaactgggcttgtcgagacagagaagactcttgcgtttctgataggcacctattggtcttactgacatccactttgcctttctctccacaggctagcctcgagaattcacgcgtggtacctctagagtcgaccgatatcactagtgccaccatgtggacattggggcggagggcagtggcgggtcttcttgcgtctcccagcccagcacaggcacaaacattgactagagttccccggccagcggagttggcccctctctgtggacggcggggactgcggacggatatagacgccacctgcacacctcgaagagctagttcaaatcagcggggcctcaatcaaatctggaacgttaagaagcagagtgtgtaccttatgaacttgagaaaaagcggaaccctcggccacccagggtcattggatgaaacaacctatgagaggcttgcggaagagacattggatagcttggccgaattctttgaagaccttgccgacaaaccctatacatttgaggattacgatgtctccttcggctctggtgtcctgactgtgaagttggggggcgacctcggaacgtacgtaataaataagcagactccgaataaacaaatttggttgtcctcaccaagtagcggccccaagcggtatgattggactgggaagaactgggtatactcccacgacggcgttagcctgcacgaactgttggcagccgagcttacaaaagctttgaagacaaaactggacctcagttctttggcctattcagggaaagacgcatagtagtctagagatatcgcggccgcttcggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcatgcaggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgcctgca

SEQ ID NO: 8 - DNA, 5999 bp, Artificial sequence
  Synthetic construct
ctgcgcgctcgctcgctcactgaggccgcccgggcaaagcccgggcgtcgggcgacctttggtcgcccggcctcagtgagcgagcgagcgcgcagagagggagtggaattcacgcgtggtacgatctgaattcggtacaattcacgcgtggtaccgatcttaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgagataaccagggctgaaagaggcccgcctgggggctggagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgtccgccgcctcctccgtgcgcccgccagcctcgcccgcgccgtcaccgtgaggcactgggcaggtaagtatcaaagtatcaaggttacaagacaggtttaaggagaccaatagaaactgggcttgtcgagacagagaagactcttgcgtttctgataggcacctattggtcttactgacatccactttgcctttctctccacaggctagcctcgagaattcacgcgtggtacctctagagtcgaccagtctcccttgggtcaggggtcctggttgcactccgtgctttgcacaaagcaggctctccatttttgttaaatgcacgaatagtgctaagctgggaagttcttcctgaggtctaacctctagctgctcccccacagaagagtgcctgcggccagtggccaccaggggtcgccgcagcacccagcgctggagggcggagcgggcggcagacccggagcagcgccaccatgtggacattggggcggagggcagtggcgggtcttcttgcgtctcccagcccagcacaggcacaaacattgactagagttccccggccagcggagttggcccctctctgtggacggcggggactgcggacggatatagacgccacctgcacacctcgaagagctagttcaaatcagcggggcctcaatcaaatctggaacgttaagaagcagagtgtgtaccttatgaacttgagaaaaagcggaaccctcggccacccagggtcattggatgaaacaacctatgagaggcttgcggaagagacattggatagcttggccgaattctttgaagaccttgccgacaaaccctatacatttgaggattacgatgtctccttcggctctggtgtcctgactgtgaagttggggggcgacctcggaacgtacgtaataaataagcagactccgaataaacaaatttggttgtcctcaccaagtagcggccccaagcggtatgattggactgggaagaactgggtatactcccacgacggcgttagcctgcacgaactgttggcagccgagcttacaaaagctttgaagacaaaactggacctcagttctttggcctattcagggaaagacgcatagtagtctagagatatcgcggccgcttcggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcatgcaggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgcctgca

SEQ ID NO: 9 - DNA, 5833 bp, Artificial sequence
  Synthetic construct
ctgcgcgctcgctcgctcactgaggccgcccgggcaaagcccgggcgtcgggcgacctttggtcgcccggcctcagtgagcgagcgagcgcgcagagagggagtggaattcacgcgtggtacgatctgaattcggtacaattcacgcgtggtaccgatcttaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgagataaccagggctgaaagaggcccgcctgggggctggagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgtccgccgcctcctccgtgcgcccgccagcctcgcccgcgccgtcaccgtgaggcactgggcaggtaagtatcaaagtatcaaggttacaagacaggtttaaggagaccaatagaaactgggcttgtcgagacagagaagactcttgcgtttctgataggcacctattggtcttactgacatccactttgcctttctctccacaggctagcctcgagaattcacgcgtggtacctctagagtcgaccgatatcactagtgccaccatgtggacattggggcggagggcagtggcgggtcttcttgcgtctcccagcccagcacaggcacaaacattgactagagttccccggccagcggagttggcccctctctgtggacggcggggactgcggacggatatagacgccacctgcacacctcgaagagctagttcaaatcagcggggcctcaatcaaatctggaacgttaagaagcagagtgtgtaccttatgaacttgagaaaaagcggaaccctcggccacccagggtcattggatgaaacaacctatgagaggcttgcggaagagacattggatagcttggccgaattctttgaagaccttgccgacaaaccctatacatttgaggattacgatgtctccttcggctctggtgtcctgactgtgaagttggggggcgacctcggaacgtacgtaataaataagcagactccgaataaacaaatttggttgtcctcaccaagtagcggccccaagcggtatgattggactgggaagaactgggtatactcccacgacggcgttagcctgcacgaactgttggcagccgagcttacaaaagctttgaagacaaaactggacctcagttctttggcctattcagggaaagacgcaggtaagcctatccctaaccctctcctcggtctcgattctacgtagtagtctagagatatcgcggccgcttcggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcatgcaggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgcctgca

SEQ ID NO: 10 - DNA, 6041 bp, Artificial sequence
  Synthetic construct
ctgcgcgctcgctcgctcactgaggccgcccgggcaaagcccgggcgtcgggcgacctttggtcgcccggcctcagtgagcgagcgagcgcgcagagagggagtggaattcacgcgtggtacgatctgaattcggtacaattcacgcgtggtaccgatcttaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgagataaccagggctgaaagaggcccgcctgggggctggagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgtccgccgcctcctccgtgcgcccgccagcctcgcccgcgccgtcaccgtgaggcactgggcaggtaagtatcaaagtatcaaggttacaagacaggtttaaggagaccaatagaaactgggcttgtcgagacagagaagactcttgcgtttctgataggcacctattggtcttactgacatccactttgcctttctctccacaggctagcctcgagaattcacgcgtggtacctctagagtcgaccagtctcccttgggtcaggggtcctggttgcactccgtgctttgcacaaagcaggctctccatttttgttaaatgcacgaatagtgctaagctgggaagttcttcctgaggtctaacctctagctgctcccccacagaagagtgcctgcggccagtggccaccaggggtcgccgcagcacccagcgctggagggcggagcgggcggcagacccggagcagcgccaccatgtggacattggggcggagggcagtggcgggtcttcttgcgtctcccagcccagcacaggcacaaacattgactagagttccccggccagcggagttggcccctctctgtggacggcggggactgcggacggatatagacgccacctgcacacctcgaagagctagttcaaatcagcggggcctcaatcaaatctggaacgttaagaagcagagtgtgtaccttatgaacttgagaaaaagcggaaccctcggccacccagggtcattggatgaaacaacctatgagaggcttgcggaagagacattggatagcttggccgaattctttgaagaccttgccgacaaaccctatacatttgaggattacgatgtctccttcggctctggtgtcctgactgtgaagttggggggcgacctcggaacgtacgtaataaataagcagactccgaataaacaaatttggttgtcctcaccaagtagcggccccaagcggtatgattggactgggaagaactgggtatactcccacgacggcgttagcctgcacgaactgttggcagccgagcttacaaaagctttgaagacaaaactggacctcagttctttggcctattcagggaaagacgcaggtaagcctatccctaaccctctcctcggtctcgattctacgtagtagtctagagatatcgcggccgcttcggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcatgcaggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgcctgca

SEQ ID NO: 11 - DNA, 5904 bp, Artificial sequence
  Synthetic construct
cgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcctctagattcgaccagtctcccttgggtcaggggtcctggttgcactccgtgctttgcacaaagcaggctctccatttttgttaaatgcacgaatagtgctaagctgggaagttcttcctgaggtctaacctctagctgctcccccacagaagagtgcctgcggccagtggccaccaggggtcgccgcagcacccagcgctggagggcggagcgggcggcagacccggagcagcgccaccatgtggacattggggcggagggcagtggcgggtcttcttgcgtctcccagcccagcacaggcacaaacattgactagagttccccggccagcggagttggcccctctctgtggacggcggggactgcggacggatatagacgccacctgcacacctcgaagagctagttcaaatcagcggggcctcaatcaaatctggaacgttaagaagcagagtgtgtaccttatgaacttgagaaaaagcggaaccctcggccacccagggtcattggatgaaacaacctatgagaggcttgcggaagagacattggatagcttggccgaattctttgaagaccttgccgacaaaccctatacatttgaggattacgatgtctccttcggctctggtgtcctgactgtgaagttggggggcgacctcggaacgtacgtaataaataagcagactccgaataaacaaatttggttgtcctcaccaagtagcggccccaagcggtatgattggactgggaagaactgggtatactcccacgacggcgttagcctgcacgaactgttggcagccgagcttacaaaagctttgaagacaaaactggacctcagttctttggcctattcagggaaagacgcatagtagtctagagatatcgcggccgcttcggagct

SEQ ID NO: 12 - DNA, 5682 bp, Artificial sequence
  Synthetic construct
cgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgatatcactagtgccaccatgtggacattggggcggagggcagtggcgggtcttcttgcgtctcccagcccagcacaggcacaaacattgactagagttccccggccagcggagttggcccctctctgtggacggcggggactgcggacggatatagacgccacctgcacacctcgaagagctagttcaaatcagcggggcctcaatcaaatctggaacgttaagaagcagagtgtgtaccttatgaacttgagaaaaagcggaaccctcggccacccagggtcattggatgaaacaacctatgagaggcttgcggaagagacattggatagcttggccgaattctttgaagaccttgccgacaaaccctatacatttgaggattacgatgtctccttcggctctggtgtcctgactgtgaagttggggggcgacctcggaacgtacgtaataaataagcagactccgaataaacaaatttggttgtcctcaccaagtagcggccccaagcggtatgattggactgggaagaactgggtatactcccacgacggcgttagcctgcacgaactgttggcagccgagcttacaaaagctttgaagacaaaactggacctcagttctttggcctattcagggaaagacgcatagtagtctagagatatcgcggccgcttcggagct

SEQ ID NO: 13 - DNA, 5867 bp, Artificial sequence
  Synthetic construct
ctgcgcgctcgctcgctcactgaggccgcccgggcaaagcccgggcgtcgggcgacctttggtcgcccggcctcagtgagcgagcgagcgcgcagagagggagtggaattcacgcgtggtacgatctgaattcggtacaattccgatcttaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgagataaccagggctgaaagaggcccgcctgggggctggagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgcagtctcccttgggtcaggggtcctggttgcactccgtgctttgcacaaagcaggctctccatttttgttaaatgcacgaatagtgctaagctgggaagttcttcctgaggtctaacctctagctgctcccccacagaagagtgcctgcggccagtggccaccaggggtcgccgcagcacccagcgctggagggcggagcgggcggcagacccggagcagccaggtaagtatcaaagtatcaaggttacaagacaggtttaaggagaccaatagaaactgggcttgtcgagacagagaagactcttgcgtttctgataggcacctattggtcttactgacatccactttgcctttctctccacaggctagccttatcactagtgccaccatgtggacattggggcggagggcagtggcgggtcttcttgcgtctcccagcccagcacaggcacaaacattgactagagttccccggccagcggagttggcccctctctgtggacggcggggactgcggacggatatagacgccacctgcacacctcgaagagctagttcaaatcagcggggcctcaatcaaatctggaacgttaagaagcagagtgtgtaccttatgaacttgagaaaaagcggaaccctcggccacccagggtcattggatgaaacaacctatgagaggcttgcggaagagacattggatagcttggccgaattctttgaagaccttgccgacaaaccctatacatttgaggattacgatgtctccttcggctctggtgtcctgactgtgaagttggggggcgacctcggaacgtacgtaataaataagcagactccgaataaacaaatttggttgtcctcaccaagtagcggccccaagcggtatgattggactgggaagaactgggtatactcccacgacggcgttagcctgcacgaactgttggcagccgagcttacaaaagctttgaagacaaaactggacctcagttctttggcctattcagggaaagacgcatagtagtctagagatatcgcggccgcttcggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcatgcaggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgcctgca

SEQ ID NO: 14 - DNA, 2219 bp, Artificial sequence
  Synthetic construct
ctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcctctagattcgaccagtctcccttgggtcaggggtcctggttgcactccgtgctttgcacaaagcaggctctccatttttgttaaatgcacgaatagtgctaagctgggaagttcttcctgaggtctaacctctagctgctcccccacagaagagtgcctgcggccagtggccaccaggggtcgccgcagcacccagcgctggagggcggagcgggcggcagacccggagcagcgccaccatgtggacattggggcggagggcagtggcgggtcttcttgcgtctcccagcccagcacaggcacaaacattgactagagttccccggccagcggagttggcccctctctgtggacggcggggactgcggacggatatagacgccacctgcacacctcgaagagctagttcaaatcagcggggcctcaatcaaatctggaacgttaagaagcagagtgtgtaccttatgaacttgagaaaaagcggaaccctcggccacccagggtcattggatgaaacaacctatgagaggcttgcggaagagacattggatagcttggccgaattctttgaagaccttgccgacaaaccctatacatttgaggattacgatgtctccttcggctctggtgtcctgactgtgaagttggggggcgacctcggaacgtacgtaataaataagcagactccgaataaacaaatttggttgtcctcaccaagtagcggccccaagcggtatgattggactgggaagaactgggtatactcccacgacggcgttagcctgcacgaactgttggcagccgagcttacaaaagctttgaagacaaaactggacctcagttctttggcctattcagggaaagacgcatagtagtctagagatatcgcggccgcttcggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 15 - DNA, 2192 bp, Artificial sequence
  Synthetic construct
ctgcgcgctcgctcgctcactgaggccgcccgggcaaagcccgggcgtcgggcgacctttggtcgcccggcctcagtgagcgagcgagcgcgcagagagggagtggaattcacgcgtggtacgatctgaattcggtacaattccgatcttaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgagataaccagggctgaaagaggcccgcctgggggctggagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgcagtctcccttgggtcaggggtcctggttgcactccgtgctttgcacaaagcaggctctccatttttgttaaatgcacgaatagtgctaagctgggaagttcttcctgaggtctaacctctagctgctcccccacagaagagtgcctgcggccagtggccaccaggggtcgccgcagcacccagcgctggagggcggagcgggcggcagacccggagcagccaggtaagtatcaaagtatcaaggttacaagacaggtttaaggagaccaatagaaactgggcttgtcgagacagagaagactcttgcgtttctgataggcacctattggtcttactgacatccactttgcctttctctccacaggctagccttatcactagtgccaccatgtggacattggggcggagggcagtggcgggtcttcttgcgtctcccagcccagcacaggcacaaacattgactagagttccccggccagcggagttggcccctctctgtggacggcggggactgcggacggatatagacgccacctgcacacctcgaagagctagttcaaatcagcggggcctcaatcaaatctggaacgttaagaagcagagtgtgtaccttatgaacttgagaaaaagcggaaccctcggccacccagggtcattggatgaaacaacctatgagaggcttgcggaagagacattggatagcttggccgaattctttgaagaccttgccgacaaaccctatacatttgaggattacgatgtctccttcggctctggtgtcctgactgtgaagttggggggcgacctcggaacgtacgtaataaataagcagactccgaataaacaaatttggttgtcctcaccaagtagcggccccaagcggtatgattggactgggaagaactgggtatactcccacgacggcgttagcctgcacgaactgttggcagccgagcttacaaaagctttgaagacaaaactggacctcagttctttggcctattcagggaaagacgcatagtagtctagagatatcgcggccgcttcggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 16 - DNA, 35 bp, Homo sapiens
gtgcctgcggccagtggccaccaggggtcgccgca

SEQ ID NO: 17 - DNA, 9 bp, Homo sapiens
aatagtgct

SEQ ID NO: 18 - DNA, 14 bp, Homo sapiens
cgacccctggtggc

SEQ ID NO: 19 - DNA, 20 bp, Homo sapiens
gtggccaccaggggtcgccg

SEQ ID NO: 20 - DNA, 19 bp, Homo sapiens
tggccaccaggggtcgccg

SEQ ID NO: 21 - DNA, 20 bp, Homo sapiens
tggccaccaggggtcgccgc

SEQ ID NO: 22 - DNA, 86 bp, Homo sapiens
gtctcccctcgccgcatccactctccggccggccgcctgtccgccgcctcctccgtgcgcccgccagcctcgcccgcgccgtcacc

SEQ ID NO: 23 - DNA, 156 bp, Homo sapiens
cgggatcagccaccgcggtggcggcctagagtcgacgaggaactgaaaaaccagaaagttaactgcctgtacggaagtgttacttctgctctaaaagctgcggaattgtacccgcggccgatccaccggtcctctagattcgaccagtctcccttg

SEQ ID NO: 24 - DNA, 5867 bp, Artificial sequence; identical to SEQ ID NO: 13
  Synthetic construct

SEQ ID NO: 25 - DNA, 5882 bp, Artificial sequence
  synthetic construct
cgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgaccagtctcccttgggtcaggggtcctggttgcactccgtgctttgcacaaagcaggctctccatttttgttaaatgcacgaatagtgctaagctgggaagttcttcctgaggtctaacctctagctgctcccccacagaagagtgcctgcggccagtggccaccaggggtcgccgcagcacccagcgctggagggcggagcgggcggcagacccggagcagcggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgatatcactagtgccaccatgtggaatgtggacattggggcggagggcagtggcgggtcttcttgcgtctcccagcccagcacaggcacaaacattgactagagttccccggccagcggagttggcccctctctgtggacggcggggactgcggacggatatagacgccacctgcacacctcgaagagctagttcaaatcagcggggcctcaatcaaatctggaacgttaagaagcagagtgtgtaccttatgaacttgagaaaaagcggaaccctcggccacccagggtcattggatgaaacaacctatgagaggcttgcggaagagacattggatagcttggccgaattctttgaagaccttgccgacaaaccctatacatttgaggattacgatgtctccttcggctctggtgtcctgactgtgaagttggggggcgacctcggaacgtacgtaataaataagcagactccgaataaacaaatttggttgtcctcaccaagtagcggccccaagcggtatgattggactgggaagaactgggtatactcccacgacggcgttagcctgcacgaactgttggcagccgagcttacaaaagctttgaagacaaaactggacctcagttctttggcctattcagggaaagacgcatagtagtctagagatatcgcggccgcttcggagct

SEQ ID NO: 26 - DNA, 5689 bp, Artificial sequence
  Synthetic construct
cgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgatatcactagtgccaccatgtggaatgtggacattggggcggagggcagtggcgggtcttcttgcgtctcccagcccagcacaggcacaaacattgactagagttccccggccagcggagttggcccctctctgtggacggcggggactgcggacggatatagacgccacctgcacacctcgaagagctagttcaaatcagcggggcctcaatcaaatctggaacgttaagaagcagagtgtgtaccttatgaacttgagaaaaagcggaaccctcggccacccagggtcattggatgaaacaacctatgagaggcttgcggaagagacattggatagcttggccgaattctttgaagaccttgccgacaaaccctatacatttgaggattacgatgtctccttcggctctggtgtcctgactgtgaagttggggggcgacctcggaacgtacgtaataaataagcagactccgaataaacaaatttggttgtcctcaccaagtagcggccccaagcggtatgattggactgggaagaactgggtatactcccacgacggcgttagcctgcacgaactgttggcagccgagcttacaaaagctttgaagacaaaactggacctcagttctttggcctattcagggaaagacgcatagtagtctagagatatcgcggccgcttcggagct

SEQ ID NO: 27 - DNA, 5780 bp, Artificial sequence
  Synthetic construct
ctgcgcgctcgctcgctcactgaggccgcccgggcaaagcccgggcgtcgggcgacctttggtcgcccggcctcagtgagcgagcgagcgcgcagagagggagtggaattcacgcgtggtacgatctgaattcggtacaattccgatcttaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgagataaccagggctgaaagaggcccgcctgggggctggagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgtccgccgcctcctccgtgcgcccgccagcctcgcccgcgccgtcaccgtgaggcactgggcaggtaagtatcaaagtatcaaggttacaagacaggtttaaggagaccaatagaaactgggcttgtcgagacagagaagactcttgcgtttctgataggcacctattggtcttactgacatccactttgcctttctctccacaggctagcctcgagaattcacgcgtggtacctctagagtcgaccgatatcactagtgccaccatgtggacattggggcggagggcagtggcgggtcttcttgcgtctcccagcccagcacaggcacaaacattgactagagttccccggccagcggagttggcccctctctgtggacggcggggactgcggacggatatagacgccacctgcacacctcgaagagctagttcaaatcagcggggcctcaatcaaatctggaacgttaagaagcagagtgtgtaccttatgaacttgagaaaaagcggaaccctcggccacccagggtcattggatgaaacaacctatgagaggcttgcggaagagacattggatagcttggccgaattctttgaagaccttgccgacaaaccctatacatttgaggattacgatgtctccttcggctctggtgtcctgactgtgaagttggggggcgacctcggaacgtacgtaataaataagcagactccgaataaacaaatttggttgtcctcaccaagtagcggccccaagcggtatgattggactgggaagaactgggtatactcccacgacggcgttagcctgcacgaactgttggcagccgagcttacaaaagctttgaagacaaaactggacctcagttctttggcctattcagggaaagacgcatagtagtctagagatatcgcggccgcttcggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcatgcaggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgcctgca

SEQ ID NO: 28 - DNA, 7184 bp, Artificial sequence
  Synthetic construct
cgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcctctagattcgaccagtctcccttgccaccatgtggacattggggcggagggcagtggcgggtcttcttgcgtctcccagcccagcacaggcacaaacattgactagagttccccggccagcggagttggcccctctctgtggacggcggggactgcggacggatatagacgccacctgcacacctcgaagagctagttcaaatcagcggggcctcaatcaaatctggaacgttaagaagcagagtgtgtaccttatgaacttgagaaaaagcggaaccctcggccacccagggtcattggatgaaacaacctatgagaggcttgcggaagagacattggatagcttggccgaattctttgaagaccttgccgacaaaccctatacatttgaggattacgatgtctccttcggctctggtgtcctgactgtgaagttggggggcgacctcggaacgtacgtaataaataagcagactccgaataaacaaatttggttgtcctcaccaagtagcggccccaagcggtatgattggactgggaagaactgggtatactcccacgacggcgttagcctgcacgaactgttggcagccgagcttacaaaagctttgaagacaaaactggacctcagttctttggcctattcagggaaagacgcatagtagtctaaagaaggaaaaattccaggagggaaaatgaattgtcttcactcttcattctttgaaggatttactgcaagaagtacatgaagagcagctggtcaacctgctcactgttctatctccaaatgagacacattaaagggtagcctacaaatgttttcaggcttctttcaaagtgtaagcacttctgagctctttagcattgaagtgtcgaaagcaactcacacgggaagatcatttcttatttgtgctctgtgactgccaaggtgtggcctgcactgggttgtccagggagacatgcatctagtgctgtttctcccacatattcacatacgtgtctgtgtgtatatatattttttcaatttaaaggttagtatggaatcagctgctacaagaatgcaaaaaatcttccaaagacaagaaaagaggaaaaaaagccgttttcatgagctgagtgatgtagcgtaacaaacaaaatcatggagctgaggaggtgccttgtaaacatgaaggggcagataaaggaaggagatactcatgttgataaagagagccctggtcctagacatagttcagccacaaagtagttgtccctttgtggacaagtttcccaaattccctggacctctgcttccccatctgttaaatgagagaatagagtatggttgattcccagcattcagtggtcctgtcaagcaacctaacaggctagttctaattccctattgggtagatgaggggatgacaaagaacagtttttaagctatataggaaacattgttattggtgttgccctatcgtgatttcagttgaattcatgtgaaaataatagccatccttggcctggcgcggtggctcacacctgtaatcccagcacttttggaggccaaggtgggtggatcacctgaggtcaggagttcaagaccagcctggccaacatgatgaaaccccgtctctactaaaaatacaaaaaattagccgggcatgatggcaggtgcctgtaatcccagctacttgggaggctgaagcggaagaatcgcttgaacccagaggtggaggttgcagtgagccgagatcgtgccattgcactgtaacctgggtgactgagcaaaactctgtctcaaaataataataacaatataataataataatagccatcctttattgtacccttactgggttaatcgtattataccacattacctcattttaatttttactgacctgcactttatacaaagcaacaagcctccaggacattaaaattcatgcaaagttatgctcatgttatattattttcttacttaaagaaggatttattagtggctgggcatggtggcgtgcacctgtaatcccaggtactcaggaggctgagacgggagaattgcttgaccccaggcggaggaggttacagtgagtcgagatcgtacctgagcgacagagcgagactccgtctcaaaaaaaaaaaaaaggagggtttattaatgagaagtttggagatatcgcggccgcttcggagct

SEQ ID NO: 29 - DNA, 5763 bp, Artificial sequence
  Synthetic construct
agcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatca

SEQ ID NO: 30 - DNA, 16 bp, Artificial sequence
  Synthetic construct
gccagccatctgttgt

SEQ ID NO: 31 - DNA, 16 bp, Artificial sequence
  Synthetic construct
ggagtggcaccttcca

SEQ ID NO: 32 - DNA, 21 bp, Artificial sequence
  Synthetic construct
tcccccgtgccttccttgacc

SEQ ID NO: 33 - DNA, 221 bp, Artificial sequence; identical to SEQ ID NO: 2
  Synthetic construct

SEQ ID NO: 34 - DNA, 280 bp, Artificial sequence
  Synthetic construct
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctac

SEQ ID NO: 35 - DNA, 1490 bp, Artificial sequence
  synthetic construct
aagaaggaaaaattccaggagggaaaatgaattgtcttcactcttcattctttgaaggatttactgcaagaagtacatgaagagcagctggtcaacctgctcactgttctatctccaaatgagacacattaaagggtagcctacaaatgttttcaggcttctttcaaagtgtaagcacttctgagctctttagcattgaagtgtcgaaagcaactcacacgggaagatcatttcttatttgtgctctgtgactgccaaggtgtggcctgcactgggttgtccagggagacatgcatctagtgctgtttctcccacatattcacatacgtgtctgtgtgtatatatattttttcaatttaaaggttagtatggaatcagctgctacaagaatgcaaaaaatcttccaaagacaagaaaagaggaaaaaaagccgttttcatgagctgagtgatgtagcgtaacaaacaaaatcatggagctgaggaggtgccttgtaaacatgaaggggcagataaaggaaggagatactcatgttgataaagagagccctggtcctagacatagttcagccacaaagtagttgtccctttgtggacaagtttcccaaattccctggacctctgcttccccatctgttaaatgagagaatagagtatggttgattcccagcattcagtggtcctgtcaagcaacctaacaggctagttctaattccctattgggtagatgaggggatgacaaagaacagtttttaagctatataggaaacattgttattggtgttgccctatcgtgatttcagttgaattcatgtgaaaataatagccatccttggcctggcgcggtggctcacacctgtaatcccagcacttttggaggccaaggtgggtggatcacctgaggtcaggagttcaagaccagcctggccaacatgatgaaaccccgtctctactaaaaatacaaaaaattagccgggcatgatggcaggtgcctgtaatcccagctacttgggaggctgaagcggaagaatcgcttgaacccagaggtggaggttgcagtgagccgagatcgtgccattgcactgtaacctgggtgactgagcaaaactctgtctcaaaataataataacaatataataataataatagccatcctttattgtacccttactgggttaatcgtattataccacattacctcattttaatttttactgacctgcactttatacaaagcaacaagcctccaggacattaaaattcatgcaaagttatgctcatgttatattattttcttacttaaagaaggatttattagtggctgggcatggtggcgtgcacctgtaatcccaggtactcaggaggctgagacgggagaattgcttgaccccaggcggaggaggttacagtgagtcgagatcgtacctgagcgacagagcgagactccgtctcaaaaaaaaaaaaaaggagggtttattaatgagaagtttg

SEQ ID NO: 36 - DNA, 141 bp, Artificial sequence
  Synthetic construct
gtaagtatcaaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 37 - DNA, 97 bp, Artificial sequence
  Synthetic construct
gtaagtttagtctttttgtcttttatttcaggtcccggatccggtggtggtgcaaatcaaagaactgctcctcagtggatgttgcctttacttctag

SEQ ID NO: 38 - DNA, 106 bp, Artificial sequence
  Synthetic construct
ctgcgcgctcgctcgctcactgaggccgcccgggcaaagcccgggcgtcgggcgacctttggtcgcccggcctcagtgagcgagcgagcgcgcagagagggagtgg

SEQ ID NO: 39 - DNA, 141 bp, Artificial sequence
  Synthetic construct
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 40 - DNA, 2457 bp, Artificial sequence
  Synthetic construct
actagtgccaccatgtggacactggggagaagggccgtggctggactgctggcttctccatctccagcccaggcccagaccctgaccagagtgcctagacctgccgaactggcccctctgtgtggcagaagaggcctgagaaccgacatcgacgccacctgtacccccagaagggccagcagcaatcagcggggcctgaatcagatctggaacgtgaagaaacagagcgtgtacctgatgaacctgagaaagagcggcaccctgggccaccctggaagcctggatgagacaacctacgagcggctggccgaggaaaccctggattccctggccgagttcttcgaggacctggccgacaagccctacaccttcgaggattacgacgtgtccttcggcagcggcgtgctgacagtgaagctgggcggagatctgggcacctacgtgatcaacaagcagacccccaacaaacagatctggctgagcagccccagcagcggccccaagagatacgattggaccggcaagaactgggtgttcagccacgacggcgtgtccctgcatgagctgctggctgccgagctgaccaaggccctgaaaacaaagctggacctgagctggctggcctacagcggcaaagatgccatcgatatccccagccccgttttaaggacattaaaagctatcaggccaagaccccagcttcattatgcagctgaggtctgttttttgttgttgttgttgtttattttttttattcctgcttttgaggacagttgggctatgtgtcacagctctgtagaaagaatgtgttgcctcctaccttgcccccaagttctgatttttaatttctatggaagattttttggattgtcggatttcctccctcacatgataccccttatcttttataatgtcttatgcctatacctgaatataacaacctttaaaaaagcaaaataataagaaggaaaaattccaggagggaaaatgaattgtcttcactcttcattctttgaaggatttactgcaagaagtacatgaagagcagctggtcaacctgctcactgttctatctccaaatgagacacattaaagggtagcctacaaatgttttcaggcttctttcaaagtgtaagcacttctgagctctttagcattgaagtgtcgaaagcaactcacacgggaagatcatttcttatttgtgctctgtgactgccaaggtgtggcctgcactgggttgtccagggagacatgcatctagtgctgtttctcccacatattcacatacgtgtctgtgtgtatatatattttttcaatttaaaggttagtatggaatcagctgctacaagaatgcaaaaaatcttccaaagacaagaaaagaggaaaaaaagccgttttcatgagctgagtgatgtagcgtaacaaacaaaatcatggagctgaggaggtgccttgtaaacatgaaggggcagataaaggaaggagatactcatgttgataaagagagccctggtcctagacatagttcagccacaaagtagttgtccctttgtggacaagtttcccaaattccctggacctctgcttccccatctgttaaatgagagaatagagtatggttgattcccagcattcagtggtcctgtcaagcaacctaacaggctagttctaattccctattgggtagatgaggggatgacaaagaacagtttttaagctatataggaaacattgttattggtgttgccctatcgtgatttcagttgaattcatgtgaaaataatagccatccttggcctggcgcggtggctcacacctgtaatcccagcacttttggaggccaaggtgggtggatcacctgaggtcaggagttcaagaccagcctggccaacatgatgaaaccccgtctctactaaaaatacaaaaaattagccgggcatgatggcaggtgcctgtaatcccagctacttgggaggctgaagcggaagaatcgcttgaacccagaggtggaggttgcagtgagccgagatcgtgccattgcactgtaacctgggtgactgagcaaaactctgtctcaaaataataataacaatataataataataatagccatcctttattgtacccttactgggttaatcgtattataccacattacctcattttaatttttactgacctgcactttatacaaagcaacaagcctccaggacattaaaattcatgcaaagttatgctcatgttatattattttcttacttaaagaaggatttattagtggctgggcatggtggcgtgcacctgtaatcccaggtactcaggaggctgagacgggagaattgcttgaccccaggcggaggaggttacagtgagtcgagatcgtacctgagcgacagagcgagactccgtctcaaaaaaaaaaaaaaggagggtttattaatgagaagtttggtcgac

SEQ ID NO: 41 - DNA, 1490 bp, Artificial sequence; identical to SEQ ID NO: 35
  Synthetic construct

SEQ ID NO: 42 - DNA, 1220 bp, Artificial sequence
  Synthetic construct
aagaaaactttcacaatttgcatccctttgtaatatgtaacagaaataaaattctcttttaaaatctatcaacaataggcaaggcacggtggctcacgcctgtcgtctcagcactttgtgaggcccaggcgggcagatcgtttgagcctagaagttcaagaccaccctgggcaacatagcgaaaccccctttctacaaaaaatacaaaaactagctgggtgtggtggtgcacacctgtagtcccagctacttggaaggctgaaatgggaagactgcttgagcccgggagggagaagttgcagtaagccaggaccacaccactgcactccagcctgggcaacagagtgagactctgtctcaaacaaacaaataaatgaggcgggtggatcacgaggtcagtagatcgagaccatcctggctaacacggtgaaacccgtctctactaaaaaaaaaaaaaaatacaaaaaattagccaggcatggtggcgggcgcctgtagtcccagttactcgggaggctgaggcaggagaatggcgtgaaaccgggaggcagagcttgcagtgagccgagatcgcaccactgccctccagcctgggcgacagagcgagactccgtctcaatcaatcaatcaatcaataaaatctattaacaatatttattgtgcacttaacaggaacatgccctgtccaaaaaaaactttacagggcttaactcattttatccttaccacaatcctatgaagtaggaacttttataaaacgcattttataaacaaggcacagagaggttaattaacttgccctctggtcacacagctaggaagtgggcagagtacagatttacacaaggcatccgtctcctggccccacatacccaactgctgtaaacccataccggcggccaagcagcctcaatttgtgcatgcacccacttcccagcaagacagcagctcccaagttcctcctgtttagaattttagaagcggcgggccaccaggctgcagtctcccttgggtcaggggtcctggttgcactccgtgctttgcacaaagcaggctctccatttttgttaaatgcacgaatagtgctaagctgggaagttcttcctgaggtctaacctctagctgctcccccacagaagagtgcctgcggccagtggccaccaggggtcgccgcagcacccagcgctggagggcggagcgggcggcagacccggagcagc

SEQ ID NO: 43 - DNA, 999 bp, Artificial sequence
  Synthetic construct
aagaaaactttcacaatttgcatccctttgtaatatgtaacagaaataaaattctcttttaaaatctatcaacaataggcaaggcacggtggctcacgcctgtcgtctcagcactttgtgaggcccaggcgggcagatcgtttgagcctagaagttcaagaccaccctgggcaacatagcgaaaccccctttctacaaaaaatacaaaaactagctgggtgtggtggtgcacacctgtagtcccagctacttggaaggctgaaatgggaagactgcttgagcccgggagggagaagttgcagtaagccaggaccacaccactgcactccagcctgggcaacagagtgagactctgtctcaaacaaacaaataaatgaggcgggtggatcacgaggtcagtagatcgagaccatcctggctaacacggtgaaacccgtctctactaaaaaaaaaaaaaaatacaaaaaattagccaggcatggtggcgggcgcctgtagtcccagttactcgggaggctgaggcaggagaatggcgtgaaaccgggaggcagagcttgcagtgagccgagatcgcaccactgccctccagcctgggcgacagagcgagactccgtctcaatcaatcaatcaatcaataaaatctattaacaatatttattgtgcacttaacaggaacatgccctgtccaaaaaaaactttacagggcttaactcattttatccttaccacaatcctatgaagtaggaacttttataaaacgcattttataaacaaggcacagagaggttaattaacttgccctctggtcacacagctaggaagtgggcagagtacagatttacacaaggcatccgtctcctggccccacatacccaactgctgtaaacccataccggcggccaagcagcctcaatttgtgcatgcacccacttcccagcaagacagcagctcccaagttcctcctgtttagaattttagaagcggcgggccaccaggctg

SEQ ID NO: 44 - DNA, 861 bp, Artificial sequence
  Synthetic construct
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 45 - DNA, 810 bp, Artificial sequence
  Synthetic construct
atgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 46 - DNA, 2357 bp, Artificial sequence
  Synthetic construct
actagtgccaccatgtggacactggggagaagggccgtggctggactgctggcttctccatctccagcccaggcccagaccctgaccagagtgcctagacctgccgaactggcccctctgtgtggcagaagaggcctgagaaccgacatcgacgccacctgtacccccagaagggccagcagcaatcagcggggcctgaatcagatctggaacgtgaagaaacagagcgtgtacctgatgaacctgagaaagagcggcaccctgggccaccctggaagcctggattgtccttcggcagcggcgtgctgacagtgaagctgggcggagatctgggcacctacgtgatcaacaagcagacccccaacaaacagatctggctgagcagccccagcagcggccccaagagatacgattggaccggcaagaactgggtgttcagccacgacggcgtgtccctgcatgagctgctggctgccgagctgaccaaggccctgaaaacaaagctggacctgagctggctggcctacagcggcaaagatgccatcgatatccccagccccgttttaaggacattaaaagctatcaggccaagaccccagcttcattatgcagctgaggtctgttttttgttgttgttgttgtttattttttttattcctgcttttgaggacagttgggctatgtgtcacagctctgtagaaagaatgtgttgcctcctaccttgcccccaagttctgatttttaatttctatggaagattttttggattgtcggatttcctccctcacatgataccccttatcttttataatgtcttatgcctatacctgaatataacaacctttaaaaaagcaaaataataagaaggaaaaattccaggagggaaaatgaattgtcttcactcttcattctttgaaggatttactgcaagaagtacatgaagagcagctggtcaacctgctcactgttctatctccaaatgagacacattaaagggtagcctacaaatgttttcaggcttctttcaaagtgtaagcacttctgagctctttagcattgaagtgtcgaaagcaactcacacgggaagatcatttcttatttgtgctctgtgactgccaaggtgtggcctgcactgggttgtccagggagacatgcatctagtgctgtttctcccacatattcacatacgtgtctgtgtgtatatatattttttcaatttaaaggttagtatggaatcagctgctacaagaatgcaaaaaatcttccaaagacaagaaaagaggaaaaaaagccgttttcatgagctgagtgatgtagcgtaacaaacaaaatcatggagctgaggaggtgccttgtaaacatgaaggggcagataaaggaaggagatactcatgttgataaagagagccctggtcctagacatagttcagccacaaagtagttgtccctttgtggacaagtttcccaaattccctggacctctgcttccccatctgttaaatgagagaatagagtatggttgattcccagcattcagtggtcctgtcaagcaacctaacaggctagttctaattccctattgggtagatgaggggatgacaaagaacagtttttaagctatataggaaacattgttattggtgttgccctatcgtgatttcagttgaattcatgtgaaaataatagccatccttggcctggcgcggtggctcacacctgtaatcccagcacttttggaggccaaggtgggtggatcacctgaggtcaggagttcaagaccagcctggccaacatgatgaaaccccgtctctactaaaaatacaaaaaattagccgggcatgatggcaggtgcctgtaatcccagctacttgggaggctgaagcggaagaatcgcttgaacccagaggtggaggttgcagtgagccgagatcgtgccattgcactgtaacctgggtgactgagcaaaactctgtctcaaaataataataacaatataataataataatagccatcctttattgtacccttactgggttaatcgtattataccacattacctcattttaatttttactgacctgcactttatacaaagcaacaagcctccaggacattaaaattcatgcaaagttatgctcatgttatattattttcttacttaaagaaggatttattagtggctgggcatggtggcgtgcacctgtaatcccaggtactcaggaggctgagacgggagaattgcttgaccccaggcggaggaggttacagtgagtcgagatcgtacctgagcgacagagcgagactccgtctcaaaaaaaaaaaaaaggagggtttattaatgagaagtttggtcgac

SEQ ID NO: 47 - DNA, 2160 bp, Artificial sequence
  Synthetic construct
actagtgccaccatgtggacactggggagaagggccgtggctggactgctggcttctccatctccagcccaggcccagaccctgaccagagtgcctagacctgccgaactggcccctctgtgtggcagaagaggcctgagaaccgacatcgacgccacctgtacccccagaagggccagcagcaatcagcggggcctgaatcagatctggaacgtgaagaaacagagcgtgtacctgatgaacctgagaaagagcggcaccctgggccaccctggaagcctggatgagacagccctgaaaacaaagctggacctgagctggctggcctacagcggcaaagatgccatcgatatccccagccccgttttaaggacattaaaagctatcaggccaagaccccagcttcattatgcagctgaggtctgttttttgttgttgttgttgtttattttttttattcctgcttttgaggacagttgggctatgtgtcacagctctgtagaaagaatgtgttgcctcctaccttgcccccaagttctgatttttaatttctatggaagattttttggattgtcggatttcctccctcacatgataccccttatcttttataatgtcttatgcctatacctgaatataacaacctttaaaaaagcaaaataataagaaggaaaaattccaggagggaaaatgaattgtcttcactcttcattctttgaaggatttactgcaagaagtacatgaagagcagctggtcaacctgctcactgttctatctccaaatgagacacattaaagggtagcctacaaatgttttcaggcttctttcaaagtgtaagcacttctgagctctttagcattgaagtgtcgaaagcaactcacacgggaagatcatttcttatttgtgctctgtgactgccaaggtgtggcctgcactgggttgtccagggagacatgcatctagtgctgtttctcccacatattcacatacgtgtctgtgtgtatatatattttttcaatttaaaggttagtatggaatcagctgctacaagaatgcaaaaaatcttccaaagacaagaaaagaggaaaaaaagccgttttcatgagctgagtgatgtagcgtaacaaacaaaatcatggagctgaggaggtgccttgtaaacatgaaggggcagataaaggaaggagatactcatgttgataaagagagccctggtcctagacatagttcagccacaaagtagttgtccctttgtggacaagtttcccaaattccctggacctctgcttccccatctgttaaatgagagaatagagtatggttgattcccagcattcagtggtcctgtcaagcaacctaacaggctagttctaattccctattgggtagatgaggggatgacaaagaacagtttttaagctatataggaaacattgttattggtgttgccctatcgtgatttcagttgaattcatgtgaaaataatagccatccttggcctggcgcggtggctcacacctgtaatcccagcacttttggaggccaaggtgggtggatcacctgaggtcaggagttcaagaccagcctggccaacatgatgaaaccccgtctctactaaaaatacaaaaaattagccgggcatgatggcaggtgcctgtaatcccagctacttgggaggctgaagcggaagaatcgcttgaacccagaggtggaggttgcagtgagccgagatcgtgccattgcactgtaacctgggtgactgagcaaaactctgtctcaaaataataataacaatataataataataatagccatcctttattgtacccttactgggttaatcgtattataccacattacctcattttaatttttactgacctgcactttatacaaagcaacaagcctccaggacattaaaattcatgcaaagttatgctcatgttatattattttcttacttaaagaaggatttattagtggctgggcatggtggcgtgcacctgtaatcccaggtactcaggaggctgagacgggagaattgcttgaccccaggcggaggaggttacagtgagtcgagatcgtacctgagcgacagagcgagactccgtctcaaaaaaaaaaaaaaggagggtttattaatgagaagtttggtcgac

SEQ ID NO: 48 - DNA, 174 bp, Artificial sequence
  Synthetic construct
cagtctcccttgggtcaggggtcctggttgcactccgtgctaagctgggaagttcttcctgaggtctaacctctagctgctcccccacagaagagtgcctgcggccagtggccaccaggggtcgccgcagcacccagcgctggagggcggagcgggcggcagacccggagcagc

SEQ ID NO: 49 - DNA, 162 bp, Artificial sequence
  Synthetic construct
cagtctcccttgggtcaggggtcctggttgcactccgtgctaagctgggaagttcttcctgaggtctaacctctagctgctcccccacagaagagtgcctgcggccagtggccaccaggggtcgccgcagcacccagcgctggaggggggcggcagaccagc

SEQ ID NO: 50 - DNA, 127 bp, Artificial sequence
  Synthetic construct
cagtctcccttgggtcaggggtcctggttgcactccgtgctaagctgggaagttcttcctgaggtctaacctctagctgctcccccacagaagagcacccagcgctggaggggggcggcagaccagc

SEQ ID NO: 51 - DNA, 63 bp, Artificial sequence
  Synthetic construct
tctaacctctagctgctcccccacagaagagcacccagcgctggaggggggcggcagaccagc

SEQ ID NO: 52 - DNA, 64 bp, Artificial sequence
  Synthetic construct
cagtctcccttgggtcaggggtcctggttgcactccgtgctaagctgggaagttcttcctgagg

SEQ ID NO: 53 - DNA, 182 bp, Artificial sequence
  Synthetic construct
cagtctcccttggaggctctccatttttgttaaatgcacgaatagtgctaagctgggaagttcttcctgaggtctaacctctagctgctcccccacagaagagtgcctgcggccagtggccaccaggggtcgccgcagcacccagcgctggagggcggagcgggcggcagacccggagcagc

SEQ ID NO: 54 - DNA, 47 bp, Artificial sequence
  Synthetic construct
gtgctttgcacaaagcaggctctccatttttgttaaatgcacgaata

SEQ ID NO: 55 - DNA, 6 bp, Artificial sequence
  Synthetic construct
cggagc

SEQ ID NO: 56 - DNA, 19 bp, Artificial sequence
  Synthetic construct
gctctccatttttgttaaa

SEQ ID NO: 57 - DNA, 15 bp, Artificial sequence
  Synthetic construct
agtgcctgcggccag

SEQ ID NO: 58 - DNA, 15 bp, Artificial sequence
  Synthetic construct
tggagggcggagcgg

SEQ ID NO: 59 - DNA, 39 bp, Artificial sequence
  Synthetic construct
gtcaggggtcctggttgcactccgtgctttgcacaaagc

SEQ ID NO: 60 - protein, 210 aa, Homo sapiens
MWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 61 - DNA, 7051 bp, Artificial sequence
  Synthetic construct
aattcccatcatcaataatataccttattttggattgaagccaatatgataatgagggggtggagtttgtgacgtggcgcggggcgtgggaacggggcgggtgacgtagtagtctctagagtcctgtattagaggtcacgtgagtgttttgcgacattttgcgacaccatgtggtcacgctgggtatttaagcccgagtgagcacgcagggtctccattttgaagcgggaggtttgaacgcgcagccaccacggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtacttcttgtctagaactcaaacaacaggaggcacggcaaatacgcagactctgggcttcagccaaggtgggcctaatacaatggccaatcaggcaaagaactggctgccaggaccctgttaccgccaacaaagagtctcaacggtaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagagatggaaccccgagatccagtacacctccaactactacaaatctacaagtgtggactttgctgttaatacagaaggcgtgtactctgaaccccgccccattggcacgcgtttcctcacccgtaatctgtaattgcttgttaatcaataaaccgtttaattcgtttcagttgaactttggtctctgcggtttaaacatcgatccggacgaaacctacgtcacccgccccgttcccacgccccgcgccacgtcacaaactccaccccctcattatcatattggcttcaatccaaaataaggtatattattgatgatgcatcgctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 62 - DNA, 219 bp, Artificial sequence
  Synthetic construct
gtctcccttgggtcaggggtcctggttgcactccgtgctttgcacaaagcaggctctccatttttgttaaatgcacgaatagtgctaagctgggaagttcttcctgaggtctaacctctagctgctcccccacagaagagtgcctgcggccagtggccaccaggggtcgccgcagcacccagcgctggagggcggagcgggcggcagacccggagcagc

SEQ ID NO: 63 - DNA, 161 bp, Artificial sequence
  synthetic construct
agctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattc